SEQUENCE LISTING (89 sequences)

SEQ ID NO: 1 - protein, 319 aa, Staphylococcus aureus
  SIGNAL (1)..(26)
  mat_peptide (27)..(319)
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 2 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 3 - protein, 281 aa, Staphylococcus aureus
DIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 4 - protein, 293 aa, Artificial sequence
  Mutated sequence
  VARIANT (1)..(12)
SETEVSVRSASSDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 5 - protein, 293 aa, Artificial sequence
  Mutated sequence
  VARIANT (35)..(35)
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 6 - protein, 281 aa, Artificial sequence
  Mutated sequence
  misc_feature (23)..(23): H23L substitution
DIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 7 - protein, 293 aa, Artificial sequence
  Mutated sequence
  VARIANT (1)..(12)
  VARIANT (35)..(35)
SETEVSVRSASSDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 8 - DNA, 960 bp, Staphylococcus aureus
atgaaaacacgtatagtcagctcagtaacaacaacactattgctaggttccatattaatgaatcctgtcgctaatgccgcagattctgatattaatattaaaaccggtactacagatattggaagcaatactacagtaaaaacaggtgatttagtcacttatgataaagaaaatggcatgcacaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaattaa

SEQ ID NO: 9 - DNA, 879 bp, Staphylococcus aureus
gcagattctgatattaatattaaaaccggtactacagatattggaagcaatactacagtaaaaacaggtgatttagtcacttatgataaagaaaatggcatgcacaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaat

SEQ ID NO: 10 - DNA, 843 bp, Staphylococcus aureus
gatattggaagcaatactacagtaaaaacaggtgatttagtcacttatgataaagaaaatggcatgcacaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaat

SEQ ID NO: 11 - DNA, 879 bp, Artificial sequence
  Mutated sequence
  mutation (1)..(36)
agcgaaaccgaagtgagcgtgcgcagcgcgagcagcgatattggaagcaatactacagtaaaaacaggtgatttagtcacttatgataaagaaaatggcatgcacaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaat

SEQ ID NO: 12 - DNA, 879 bp, Artificial sequence
  Mutated sequence
  mutation (103)..(105)
gcagattctgatattaatattaaaaccggtactacagatattggaagcaatactacagtaaaaacaggtgatttagtcacttatgataaagaaaatggcatgttaaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaat

SEQ ID NO: 13 - DNA, 843 bp, Artificial sequence
  Mutated sequence
  mutation (67)..(69)
gatattggaagcaatactacagtaaaaacaggtgatttagtcacttatgataaagaaaatggcatgttaaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaat

SEQ ID NO: 14 - DNA, 879 bp, Artificial sequence
  Mutated sequence
  mutation (1)..(36)
  mutation (103)..(105)
agcgaaaccgaagtgagcgtgcgcagcgcgagcagcgatattggaagcaatactacagtaaaaacaggtgatttagtcacttatgataaagaaaatggcatgttaaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaat

SEQ ID NO: 15 - protein, 520 aa, Staphylococcus aureus
MMTLQIHTGGINLKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 16 - protein, 291 aa, Staphylococcus aureus
AQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPK

SEQ ID NO: 17 - protein, 291 aa, Artificial sequence
  Mutated sequence
  VARIANT (3)..(3)
  VARIANT (8)..(8)
  VARIANT (35)..(35)
  VARIANT (41)..(41)
  VARIANT (64)..(64)
  VARIANT (69)..(69)
  VARIANT (96)..(96)
  VARIANT (102)..(102)
  VARIANT (122)..(122)
  VARIANT (127)..(127)
  VARIANT (154)..(154)
  VARIANT (160)..(160)
  VARIANT (180)..(180)
  VARIANT (185)..(185)
  VARIANT (212)..(212)
  VARIANT (218)..(218)
  VARIANT (238)..(238)
  VARIANT (243)..(243)
  VARIANT (270)..(270)
  VARIANT (276)..(276)
AQVDEAQKNAFYQVLNMPNLNADQRNGFIQSLKDAPSQSAKVLGEAQKLNDSQAPKADAQQNNVNKDQKSAFYEILNMPNLNEAQRNGFIQSLKDAPSQSTKVLGEAKKLNESQAPKADNNVNKEQKNAFYEILNMPNLNEEQRNGFIQSLKDAPSQSAKLLSEAKKLNESQAPKADNKVNKEQKNAFYEILHLPNLNEEQRNGFIQSLKDAPSQSAKLLAEAKKLNDAQAPKADNKVNKEQKNAFYEILHLPNLTEEQRNGFIQSLKDAPSVSKKILAEAKKLNDAQAPK

SEQ ID NO: 18 - protein, 291 aa, Artificial sequence
  Mutated sequence
  VARIANT (3)..(3)
  VARIANT (8)..(8)
  VARIANT (35)..(35)
  VARIANT (37)..(38)
  VARIANT (64)..(64)
  VARIANT (69)..(69)
  VARIANT (96)..(96)
  VARIANT (98)..(99)
  VARIANT (122)..(122)
  VARIANT (127)..(127)
  VARIANT (154)..(154)
  VARIANT (156)..(157)
  VARIANT (180)..(180)
  VARIANT (185)..(185)
  VARIANT (212)..(212)
  VARIANT (214)..(215)
  VARIANT (238)..(238)
  VARIANT (243)..(243)
  VARIANT (270)..(270)
  VARIANT (272)..(273)
AQVDEAQKNAFYQVLNMPNLNADQRNGFIQSLKDAPAKSANVLGEAQKLNDSQAPKADAQQNNVNKDQKSAFYEILNMPNLNEAQRNGFIQSLKDAPAKSTNVLGEAKKLNESQAPKADNNVNKEQKNAFYEILNMPNLNEEQRNGFIQSLKDAPAKSANLLSEAKKLNESQAPKADNKVNKEQKNAFYEILHLPNLNEEQRNGFIQSLKDAPAKSANLLAEAKKLNDAQAPKADNKVNKEQKNAFYEILHLPNLTEEQRNGFIQSLKDAPAKSKEILAEAKKLNDAQAPK

SEQ ID NO: 19 - protein, 291 aa, Artificial sequence
  Mutated sequence
  VARIANT (3)..(3)
  VARIANT (8)..(8)
  VARIANT (34)..(34)
  VARIANT (37)..(37)
  VARIANT (64)..(64)
  VARIANT (69)..(69)
  VARIANT (95)..(95)
  VARIANT (98)..(98)
  VARIANT (122)..(122)
  VARIANT (127)..(127)
  VARIANT (153)..(153)
  VARIANT (156)..(156)
  VARIANT (180)..(180)
  VARIANT (185)..(185)
  VARIANT (211)..(211)
  VARIANT (214)..(214)
  VARIANT (238)..(238)
  VARIANT (243)..(243)
  VARIANT (269)..(269)
  VARIANT (272)..(272)
AQVDEAQKNAFYQVLNMPNLNADQRNGFIQSLKRDPRQSANVLGEAQKLNDSQAPKADAQQNNVNKDQKSAFYEILNMPNLNEAQRNGFIQSLKRDPRQSTNVLGEAKKLNESQAPKADNNVNKEQKNAFYEILNMPNLNEEQRNGFIQSLKRDPRQSANLLSEAKKLNESQAPKADNKVNKEQKNAFYEILHLPNLNEEQRNGFIQSLKRDPRQSANLLAEAKKLNDAQAPKADNKVNKEQKNAFYEILHLPNLTEEQRNGFIQSLKRDPRVSKEILAEAKKLNDAQAPK

SEQ ID NO: 20 - protein, 291 aa, Artificial sequence
  Mutated sequence
  VARIANT (3)..(3)
  VARIANT (8)..(8)
  VARIANT (35)..(35)
  VARIANT (37)..(37)
  VARIANT (64)..(64)
  VARIANT (69)..(69)
  VARIANT (96)..(96)
  VARIANT (98)..(98)
  VARIANT (122)..(122)
  VARIANT (127)..(127)
  VARIANT (154)..(154)
  VARIANT (156)..(156)
  VARIANT (180)..(180)
  VARIANT (185)..(185)
  VARIANT (212)..(212)
  VARIANT (214)..(214)
  VARIANT (238)..(238)
  VARIANT (243)..(243)
  VARIANT (270)..(270)
  VARIANT (272)..(272)
AQVDEAQKNAFYQVLNMPNLNADQRNGFIQSLKDRPRQSANVLGEAQKLNDSQAPKADAQQNNVNKDQKSAFYEILNMPNLNEAQRNGFIQSLKDRPRQSTNVLGEAKKLNESQAPKADNNVNKEQKNAFYEILNMPNLNEEQRNGFIQSLKDRPRQSANLLSEAKKLNESQAPKADNKVNKEQKNAFYEILHLPNLNEEQRNGFIQSLKDRPRQSANLLAEAKKLNDAQAPKADNKVNKEQKNAFYEILHLPNLTEEQRNGFIQSLKDRPRVSKEILAEAKKLNDAQAPK

SEQ ID NO: 21 - protein, 291 aa, Artificial sequence
  Mutated sequence
  VARIANT (8)..(9)
  VARIANT (35)..(35)
  VARIANT (41)..(41)
  VARIANT (69)..(70)
  VARIANT (96)..(96)
  VARIANT (102)..(102)
  VARIANT (127)..(128)
  VARIANT (154)..(154)
  VARIANT (160)..(160)
  VARIANT (185)..(186)
  VARIANT (212)..(212)
  VARIANT (218)..(218)
  VARIANT (243)..(244)
  VARIANT (270)..(270)
  VARIANT (276)..(276)
AQHDEAQKKAFYQVLNMPNLNADQRNGFIQSLKDAPSQSAKVLGEAQKLNDSQAPKADAQQNNFNKDQKKAFYEILNMPNLNEAQRNGFIQSLKDAPSQSTKVLGEAKKLNESQAPKADNNFNKEQKKAFYEILNMPNLNEEQRNGFIQSLKDAPSQSAKLLSEAKKLNESQAPKADNKFNKEQKKAFYEILHLPNLNEEQRNGFIQSLKDAPSQSAKLLAEAKKLNDAQAPKADNKFNKEQKKAFYEILHLPNLTEEQRNGFIQSLKDAPSVSKKILAEAKKLNDAQAPK

SEQ ID NO: 22 - DNA, 1563 bp, Staphylococcus aureus
atgatgactttacaaatacatacagggggtattaatttgaaaaagaaaaacatttattcaattcgtaaactaggtgtaggtattgcatctgtaactttaggtacattacttatatctggtggcgtaacacctgctgcaaatgctgcgcaacacgatgaagctcaacaaaatgctttttatcaagtcttaaatatgcctaacttaaatgctgatcaacgcaatggttttatccaaagccttaaagatgatccaagccaaagtgctaacgttttaggtgaagctcaaaaacttaatgactctcaagctccaaaagctgatgcgcaacaaaataacttcaacaaagatcaacaaagcgccttctatgaaatcttgaacatgcctaacttaaacgaagcgcaacgtaacggcttcattcaaagtcttaaagacgacccaagccaaagcactaacgttttaggtgaagctaaaaaattaaacgaatctcaagcaccgaaagctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgctcaagcaccaaaagctgacaacaaattcaacaaagaacaacaaaatgctttctatgaaattttacatttacctaacttaactgaagaacaacgtaacggcttcatccaaagccttaaagacgatccttcagtgagcaaagaaattttagcagaagctaaaaagctaaacgatgctcaagcaccaaaagaggaagacaataacaagcctggcaaagaagacaataacaagcctggcaaagaagacaacaacaagcctggtaaagaagacaacaacaagcctggcaaagaagacggcaacaagcctggtaaagaagacaacaaaaaacctggtaaagaagatggcaacaagcctggtaaagaagacaacaaaaaacctggtaaagaagacggcaacaagcctggcaaagaagatggcaacaaacctggtaaagaagatggtaacggagtacatgtcgttaaacctggtgatacagtaaatgacattgcaaaagcaaacggcactactgctgacaaaattgctgcagataacaaattagctgataaaaacatgatcaaacctggtcaagaacttgttgttgataagaagcaaccagcaaaccatgcagatgctaacaaagctcaagcattaccagaaactggtgaagaaaatccattcatcggtacaactgtatttggtggattatcattagccttaggtgcagcgttattagctggacgtcgtcgcgaactataa

SEQ ID NO: 23 - DNA, 873 bp, Staphylococcus aureus
gcgcaacacgatgaagctcaacaaaatgctttttatcaagtcttaaatatgcctaacttaaatgctgatcaacgcaatggttttatccaaagccttaaagatgatccaagccaaagtgctaacgttttaggtgaagctcaaaaacttaatgactctcaagctccaaaagctgatgcgcaacaaaataacttcaacaaagatcaacaaagcgccttctatgaaatcttgaacatgcctaacttaaacgaagcgcaacgtaacggcttcattcaaagtcttaaagacgacccaagccaaagcactaacgttttaggtgaagctaaaaaattaaacgaatctcaagcaccgaaagctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgctcaagcaccaaaagctgacaacaaattcaacaaagaacaacaaaatgctttctatgaaattttacatttacctaacttaactgaagaacaacgtaacggcttcatccaaagccttaaagacgatccttcagtgagcaaagaaattttagcagaagctaaaaagctaaacgatgctcaagcaccaaaa

SEQ ID NO: 24 - DNA, 873 bp, Artificial sequence
  Mutated sequence
gcgcaagtggatgaagctcaaaaaaatgctttttatcaagtcttaaatatgcctaacttaaatgctgatcaacgcaatggttttatccaaagccttaaagatgctccaagccaaagtgctaaagttttaggtgaagctcaaaaacttaatgactctcaagctccaaaagctgatgcgcaacaaaataacgtgaacaaagatcaaaaaagcgccttctatgaaatcttgaacatgcctaacttaaacgaagcgcaacgtaacggcttcattcaaagtcttaaagacgctccaagccaaagcactaaagttttaggtgaagctaaaaaattaaacgaatctcaagcaccgaaagctgataacaatgtgaacaaagaacaaaaaaatgctttctatgaaatcttgaatatgcctaacttaaacgaagaacaacgcaatggtttcatccaaagcttaaaagatgctccaagccaaagtgctaaactattgtcagaagctaaaaagttaaatgaatctcaagcaccgaaagcggataacaaagtgaacaaagaacaaaaaaatgctttctatgaaatcttacatttacctaacttaaacgaagaacaacgcaatggtttcatccaaagcctaaaagatgctccaagccaaagcgctaaacttttagcagaagctaaaaagctaaatgatgctcaagcaccaaaagctgacaacaaagtgaacaaagaacaaaaaaatgctttctatgaaattttacatttacctaacttaactgaagaacaacgtaacggcttcatccaaagccttaaagacgctccttcagtgagcaaaaaaattttagcagaagctaaaaagctaaacgatgctcaagcaccaaaa

SEQ ID NO: 25 - DNA, 873 bp, Artificial sequence
  Mutated sequence
gcgcaagtggatgaagctcaaaaaaatgctttttatcaagtcttaaatatgcctaacttaaatgctgatcaacgcaatggttttatccaaagccttaaagatgcaccagcaaaaagtgctaacgttttaggtgaagctcaaaaacttaatgactctcaagctccaaaagctgatgcgcaacaaaataacgtgaacaaagatcaaaaaagcgccttctatgaaatcttgaacatgcctaacttaaacgaagcgcaacgtaacggcttcattcaaagtcttaaagacgcaccagcaaaaagcactaacgttttaggtgaagctaaaaaattaaacgaatctcaagcaccgaaagctgataacaatgtgaacaaagaacaaaaaaatgctttctatgaaatcttgaatatgcctaacttaaacgaagaacaacgcaatggtttcatccaaagcttaaaagatgcaccagcaaaaagtgctaacctattgtcagaagctaaaaagttaaatgaatctcaagcaccgaaagcggataacaaagtgaacaaagaacaaaaaaatgctttctatgaaatcttacatttacctaacttaaacgaagaacaacgcaatggtttcatccaaagcctaaaagatgcaccagcaaaaagcgctaaccttttagcagaagctaaaaagctaaatgatgctcaagcaccaaaagctgacaacaaagtgaacaaagaacaaaaaaatgctttctatgaaattttacatttacctaacttaactgaagaacaacgtaacggcttcatccaaagccttaaagacgcacctgcaaaaagcaaagaaattttagcagaagctaaaaagctaaacgatgctcaagcaccaaaa

SEQ ID NO: 26 - DNA, 873 bp, Artificial sequence
  Mutated sequence
gcgcaagtggatgaagctcaaaaaaatgctttttatcaagtcttaaatatgcctaacttaaatgctgatcaacgcaatggttttatccaaagccttaaaagggatccaaggcaaagtgctaacgttttaggtgaagctcaaaaacttaatgactctcaagctccaaaagctgatgcgcaacaaaataacgtgaacaaagatcaaaaaagcgccttctatgaaatcttgaacatgcctaacttaaacgaagcgcaacgtaacggcttcattcaaagtcttaaaagggacccaaggcaaagcactaacgttttaggtgaagctaaaaaattaaacgaatctcaagcaccgaaagctgataacaatgtgaacaaagaacaaaaaaatgctttctatgaaatcttgaatatgcctaacttaaacgaagaacaacgcaatggtttcatccaaagcttaaaaagggacccaaggcaaagtgctaacctattgtcagaagctaaaaagttaaatgaatctcaagcaccgaaagcggataacaaagtgaacaaagaacaaaaaaatgctttctatgaaatcttacatttacctaacttaaacgaagaacaacgcaatggtttcatccaaagcctaaaaagggacccaaggcaaagcgctaaccttttagcagaagctaaaaagctaaatgatgctcaagcaccaaaagctgacaacaaagtgaacaaagaacaaaaaaatgctttctatgaaattttacatttacctaacttaactgaagaacaacgtaacggcttcatccaaagccttaaaagggatcctagggtgagcaaagaaattttagcagaagctaaaaagctaaacgatgctcaagcaccaaaa

SEQ ID NO: 27 - DNA, 873 bp, Artificial sequence
  Mutated sequence
gcgcaagtggatgaagctcaaaaaaatgctttttatcaagtcttaaatatgcctaacttaaatgctgatcaacgcaatggttttatccaaagccttaaagataggccaaggcaaagtgctaacgttttaggtgaagctcaaaaacttaatgactctcaagctccaaaagctgatgcgcaacaaaataacgtgaacaaagatcaaaaaagcgccttctatgaaatcttgaacatgcctaacttaaacgaagcgcaacgtaacggcttcattcaaagtcttaaagacaggccaaggcaaagcactaacgttttaggtgaagctaaaaaattaaacgaatctcaagcaccgaaagctgataacaatgtgaacaaagaacaaaaaaatgctttctatgaaatcttgaatatgcctaacttaaacgaagaacaacgcaatggtttcatccaaagcttaaaagataggccaaggcaaagtgctaacctattgtcagaagctaaaaagttaaatgaatctcaagcaccgaaagcggataacaaagtgaacaaagaacaaaaaaatgctttctatgaaatcttacatttacctaacttaaacgaagaacaacgcaatggtttcatccaaagcctaaaagataggccaaggcaaagcgctaaccttttagcagaagctaaaaagctaaatgatgctcaagcaccaaaagctgacaacaaagtgaacaaagaacaaaaaaatgctttctatgaaattttacatttacctaacttaactgaagaacaacgtaacggcttcatccaaagccttaaagacaggcctagggtgagcaaagaaattttagcagaagctaaaaagctaaacgatgctcaagcaccaaaa

SEQ ID NO: 28 - DNA, 873 bp, Artificial sequence
  Mutated sequence
gcgcaacacgatgaagctcaaaaaaaagctttttatcaagtcttaaatatgcctaacttaaatgctgatcaacgcaatggttttatccaaagccttaaagatgctccaagccaaagtgctaaagttttaggtgaagctcaaaaacttaatgactctcaagctccaaaagctgatgcgcaacaaaataacttcaacaaagatcaaaaaaaagccttctatgaaatcttgaacatgcctaacttaaacgaagcgcaacgtaacggcttcattcaaagtcttaaagacgctccaagccaaagcactaaagttttaggtgaagctaaaaaattaaacgaatctcaagcaccgaaagctgataacaatttcaacaaagaacaaaaaaaagctttctatgaaatcttgaatatgcctaacttaaacgaagaacaacgcaatggtttcatccaaagcttaaaagatgctccaagccaaagtgctaaactattgtcagaagctaaaaagttaaatgaatctcaagcaccgaaagcggataacaaattcaacaaagaacaaaaaaaagctttctatgaaatcttacatttacctaacttaaacgaagaacaacgcaatggtttcatccaaagcctaaaagatgctccaagccaaagcgctaaacttttagcagaagctaaaaagctaaatgatgctcaagcaccaaaagctgacaacaaattcaacaaagaacaaaaaaaagctttctatgaaattttacatttacctaacttaactgaagaacaacgtaacggcttcatccaaagccttaaagacgctccttcagtgagcaaaaaaattttagcagaagctaaaaagctaaacgatgctcaagcaccaaaa

SEQ ID NO: 29 - protein, 311 aa, Staphylococcus aureus
  SIGNAL (1)..(28)
  mat_peptide (29)..(311)
MFKKKMLAATLSVGLIAPLASPIQESRANTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 30 - protein, 283 aa, Staphylococcus aureus
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 31 - DNA, 936 bp, Staphylococcus aureus
atgtttaagaaaaaaatgttagctgcaactttgtcagtaggactgattgcacctttagcatctccgattcaagaatctagagcaaatactaatattgaaaatattggtgatggtgctgaagtaatcaaacgtacggaggatgtaagtagtaagaaatggggcgttactcaaaatgtccaattcgactttgtaaaagataaaaaatataacaaagacgctttaattgttaaaatgcaaggttttattaattccagaacttcattttcagatgtgaagggtagtggatatgaattaactaaacgaatgatttggccattccaatataatataggactgacgactaaagatccaaatgttagcttaatcaattaccttcctaaaaacaaaatagaaactactgatgttggtcaaacattaggatataacattggaggtaatttccagtcagcaccatctataggtggcaatggctcatttaattattctaaaacaattagttatacccaaaagagttatgtcagtgaagtagacaagcaaaactcaaaatctgttaaatggggtgttaaagcaaacgaatttgttacgcctgatggaaaaaaatctgcgcatgatagatatttattcgtacaaagtccaaatggtccaacaggttcagcaagagaatattttgctcctgataatcaattgccacctttagttcaaagtggctttaatccatcgtttatcactacactatcacatgaaaaaggttcaagtgatacgagtgaatttgaaatttcatatggtagaaacttagatattacatatgcgactttattccctagaactggtatttacgcagaaagaaagcataatgcatttgtaaatagaaactttgtagttagatatgaagttaattggaaaacacacgaaattaaagtgaaaggacataattaa

SEQ ID NO: 32 - DNA, 849 bp, Staphylococcus aureus
aatactaatattgaaaatattggtgatggtgctgaagtaatcaaacgtacggaggatgtaagtagtaagaaatggggcgttactcaaaatgtccaattcgactttgtaaaagataaaaaatataacaaagacgctttaattgttaaaatgcaaggttttattaattccagaacttcattttcagatgtgaagggtagtggatatgaattaactaaacgaatgatttggccattccaatataatataggactgacgactaaagatccaaatgttagcttaatcaattaccttcctaaaaacaaaatagaaactactgatgttggtcaaacattaggatataacattggaggtaatttccagtcagcaccatctataggtggcaatggctcatttaattattctaaaacaattagttatacccaaaagagttatgtcagtgaagtagacaagcaaaactcaaaatctgttaaatggggtgttaaagcaaacgaatttgttacgcctgatggaaaaaaatctgcgcatgatagatatttattcgtacaaagtccaaatggtccaacaggttcagcaagagaatattttgctcctgataatcaattgccacctttagttcaaagtggctttaatccatcgtttatcactacactatcacatgaaaaaggttcaagtgatacgagtgaatttgaaatttcatatggtagaaacttagatattacatatgcgactttattccctagaactggtatttacgcagaaagaaagcataatgcatttgtaaatagaaactttgtagttagatatgaagttaattggaaaacacacgaaattaaagtgaaaggacataat

SEQ ID NO: 33 - protein, 509 aa, Staphylococcus aureus
  SIGNAL (1)..(27)
  PROPEP (28)..(209)
  mat_peptide (210)..(509)
MRKFSRYAFTSMAALTLLSTLSPAALAIDSKNKPANSDIKFEVTQKSDAVKALKELPKSENVKNIYQDYAVTDVKTDKKGFTHYTLQPSVDGVHAPDKEVKVHADKSGKVVLINGDTDAKKVKPTNKVTLSKDDAADKAFKAVKIDKNKAKNLKDKVIKENKVEIDGDSNKYVYNVELITVTPEISHWKVKIDAQTGEILEKMNLVKEAAETGKGKGVLGDTKDININSIDGGFSLEDLTHQGKLSAFSFNDQTGQATLITNEDENFVKDEQRAGVDANYYAKQTYDYYKDTFGRESYDNQGSPIVSLTHVNNYGGQDNRNNAAWIGDKMIYGDGDGRTFTSLSGANDVVAHELTHGVTQETANLEYKDQSGALNESFSDVFGYFVDDEDFLMGEDVYTPGKEGDALRSMSNPEQFGQPAHMKDYVFTEKDNGGVHTNSGIPNKAAYNVIQAIGKSKSEQIYYRALTEYLTSNSNFKDCKDALYQAAKDLYDEQTAEQVYEAWNEVGVE

SEQ ID NO: 34 - protein, 482 aa, Staphylococcus aureus
IDSKNKPANSDIKFEVTQKSDAVKALKELPKSENVKNIYQDYAVTDVKTDKKGFTHYTLQPSVDGVHAPDKEVKVHADKSGKVVLINGDTDAKKVKPTNKVTLSKDDAADKAFKAVKIDKNKAKNLKDKVIKENKVEIDGDSNKYVYNVELITVTPEISHWKVKIDAQTGEILEKMNLVKEAAETGKGKGVLGDTKDININSIDGGFSLEDLTHQGKLSAFSFNDQTGQATLITNEDENFVKDEQRAGVDANYYAKQTYDYYKDTFGRESYDNQGSPIVSLTHVNNYGGQDNRNNAAWIGDKMIYGDGDGRTFTSLSGANDVVAHELTHGVTQETANLEYKDQSGALNESFSDVFGYFVDDEDFLMGEDVYTPGKEGDALRSMSNPEQFGQPAHMKDYVFTEKDNGGVHTNSGIPNKAAYNVIQAIGKSKSEQIYYRALTEYLTSNSNFKDCKDALYQAAKDLYDEQTAEQVYEAWNEVGVE

SEQ ID NO: 35 - protein, 482 aa, Artificial sequence
  Mutated sequence
  VARIANT (326)..(326)
  VARIANT (452)..(452)
IDSKNKPANSDIKFEVTQKSDAVKALKELPKSENVKNIYQDYAVTDVKTDKKGFTHYTLQPSVDGVHAPDKEVKVHADKSGKVVLINGDTDAKKVKPTNKVTLSKDDAADKAFKAVKIDKNKAKNLKDKVIKENKVEIDGDSNKYVYNVELITVTPEISHWKVKIDAQTGEILEKMNLVKEAAETGKGKGVLGDTKDININSIDGGFSLEDLTHQGKLSAFSFNDQTGQATLITNEDENFVKDEQRAGVDANYYAKQTYDYYKDTFGRESYDNQGSPIVSLTHVNNYGGQDNRNNAAWIGDKMIYGDGDGRTFTSLSGANDVVAHALTHGVTQETANLEYKDQSGALNESFSDVFGYFVDDEDFLMGEDVYTPGKEGDALRSMSNPEQFGQPAHMKDYVFTEKDNGGVHTNSGIPNKAAYNVIQAIGKSKSEQIYYRALTEYLTSNSNFKDSKDALYQAAKDLYDEQTAEQVYEAWNEVGVE

SEQ ID NO: 36 - DNA, 1446 bp, Artificial sequence
  Mutated sequence
  mutation (976)..(978)
  mutation (1354)..(1356)
attgattcaaaaaataaaccagctaattctgatattaaatttgaggtgactcaaaagagtgatgcggtcaaagcattaaaagaattgcctaaatccgaaaatgtaaaaaatatttatcaagattacgctgttactgatgtaaaaactgataaaaaaggatttacgcattatacattgcaaccgagtgttgatggtgttcatgcacctgacaaagaagtgaaagtacacgcagacaaatcaggaaaagtcgttttaatcaatggggatactgatgcgaagaaagtaaagccaacgaataaagtgacattaagtaaagatgacgcagccgacaaagcatttaaagcagttaagattgataagaataaagcgaaaaatcttaaagataaagtcattaaagaaaacaaagttgaaatcgatggtgacagtaataaatacgtttataatgttgagttaattacagtgacaccagaaatttcacattggaaagttaaaattgatgctcaaactggcgaaattttagaaaaaatgaacttagttaaagaagctgcagaaactggtaaaggaaaaggtgtacttggcgatacaaaagatatcaatatcaatagtattgacggtggatttagcctagaagatttaacgcatcaaggtaaattatcagcatttagctttaatgatcaaacaggtcaagcaacattgattactaatgaagatgaaaacttcgtaaaagatgagcaacgtgctggcgtagatgcaaattattacgctaaacaaacatatgattattacaaagacacatttggtcgtgaatcatatgacaaccaaggtagtccaattgtttcattaacgcatgttaataactacggtggtcaagataacagaaataatgccgcatggatcggtgacaaaatgatctatggtgatggtgatggtcgcacattcacaagtttatcgggtgcaaatgacgtagtagcacacgcattaacacacggtgtgacacaagagacagcgaacttagaatataaggaccagtcaggcgctctaaatgaaagcttttcagatgtttttggatactttgtagatgacgaggatttcttaatgggtgaagatgtctacacacctggaaaagagggagacgctttacgcagcatgtcaaacccagaacaatttggtcaaccagctcatatgaaagactatgtattcactgaaaaagataatggtggcgtacatacgaattctggaattccaaataaagcagcttataacgtgattcaagcaatagggaaatctaaatcagaacaaatttactaccgagcattaacggaatacttaacaagtaattcaaacttcaaagatagcaaagatgcattataccaagcggctaaagatttatatgacgagcaaacagctgaacaggtgtatgaagcatggaatgaagtaggcgtggag

SEQ ID NO: 37 - protein, 619 aa, Staphylococcus aureus
MPKNKILIYLLSTTLVLPTLVSPTAYADTPQKDTTAKTTSHDSKKSTDDETSKDTTSKDIDKADNNNTSNQDNNDKKVKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKVNQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLEADGNQLYSINAGFRKYPSTKESLKDYSDLIKNGIDGNRTIYKPTWKSEADSYKDATSHLSKTYATDPNYAKKLNSIIKHYQLTQFDDERMPDLDKYERSIKDYDDSSDEFKPFREVSDNMPYPHGQCTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGK

SEQ ID NO: 38 - protein, 592 aa, Staphylococcus aureus
DTPQKDTTAKTTSHDSKKSTDDETSKDTTSKDIDKADNNNTSNQDNNDKKVKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKVNQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLEADGNQLYSINAGFRKYPSTKESLKDYSDLIKNGIDGNRTIYKPTWKSEADSYKDATSHLSKTYATDPNYAKKLNSIIKHYQLTQFDDERMPDLDKYERSIKDYDDSSDEFKPFREVSDNMPYPHGQCTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGK

SEQ ID NO: 39 - protein, 592 aa, Artificial sequence
  Mutated sequence
  VARIANT (386)..(386)
  VARIANT (411)..(411)
  VARIANT (513)..(513)
DTPQKDTTAKTTSHDSKKSNDDETSKDTTSKDIDKADNNNTSNQDNNDKKFKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKANQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLQADGNKLYSINAGFRKYPSTKESLKNYSDLIKNGIDGNRTIYKPTWKSEADSYKDATSHLSKTYATDPNYAKKLNSIIKHYQLTQFDDERMPDLDKYERSIKDYDDSSDEFKPFREVSDSMPYPHGQSTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGK

SEQ ID NO: 40 - DNA, 1860 bp, Staphylococcus aureus
atgcctaaaaataaaattttaatttatttgctatcaactacgctcgtattacctactttagtttcacctaccgcttatgctgacacacctcaaaaagatactacagctaagacaacatctcatgattccaaaaaatctactgatgatgaaacttctaaggatactacaagtaaagatattgataaagcagacaacaataatactagtaaccaagacaataacgacaaaaaagttaaaactatagacgacagcacttcagactctaacaatatcattgattttatttataagaatttaccacaaaccaatataaaccaattgctaaccaaaaataaatacgatgataattactcattaacaactttaatccaaaacttattcaatttaaattcggatatttctgattacgaacaacctcgtaatggtgaaaagtcaacaaatgattcgaataaaaacagtgataatagcatcaaaaatgatacggatacgcaatcatctaaacaagataaagcagacaatcaaaaagcacctaaatcaaacaatacaaaaccaagtacatctaataagcaaccaaattcgccaaagccaacacaaccaaatcaatcaaatagtcaaccagcaagtgacgataaagtaaatcaaaaatcttcatcgaaagataatcaatcaatgtcagattcggctttagattctattttggatcaatacagtgaagatgcaaagaaaacacaaaaagattacgcatctcaatctaaaaaagacaaaaatgaaaaatctaatacaaagaatccacagttaccaacacaagatgaattgaaacataaatctaaacctgctcaatcattcaataacgatgttaatcaaaaggatacacgtgcaacatcactattcgaaacagatcctagtatatctaacaatgatgatagtggacaatttaacgttgttgactcaaaagatacacgtcaatttgtcaaatcaattgctaaagatgcacaccgcattggtcaagataacgatatttatgcgtctgtcatgattgcccaagcaatcttagaatctgactcaggtcgtagtgctttagctaagtcaccaaaccataatttattcggtatcaaaggtgcttttgaagggaattctgttccttttaacacattagaagctgatggtaatcaattgtatagtattaatgctggattccgaaaatatccaagcacgaaagaatcactaaaagattactctgaccttattaaaaatggtattgatggcaatcgaacaatttataaaccaacatggaaatcggaagccgattcttataaagatgcaacatcacacttatctaaaacatatgctacagatccaaactatgctaagaaattaaacagtattattaaacactatcaattaactcagtttgacgatgaacgtatgccagatttagataaatatgaacgttctatcaaggattatgatgattcatcagatgaattcaaacctttccgcgaggtatctgataatatgccatatccacatggccaatgtacttggtacgtatataaccgtatgaaacaatttggtacatctatctcaggtgatttaggtgatgcacataattggaataatcgagctcaataccgtgattatcaagtaagtcatacaccaaaacgtcatgctgctgttgtatttgaggctggacaatttggtgcagatcaacattacggtcatgtagcatttgttgaaaaagttaacagtgatggttctatcgttatttcagaatccaatgttaaaggattaggtatcatttctcatagaactatcaatgcagctgccgctgaagaattatcatatattacaggtaaataa

SEQ ID NO: 41 - DNA, 1776 bp, Artificial sequence
  Mutated sequence
gatacacctcaaaaagatactacagctaagacaacatctcatgattcaaaaaaatctaatgacgatgaaacttctaaggatactacaagtaaagatattgataaagcagacaacaataatacaagtaaccaagacaataacgacaaaaaatttaaaactatagacgacagcacttcagactctaacaatatcattgattttatttataagaatttaccacaaaccaatataaaccaattgctaaccaaaaataaatacgatgataattactcattaacaactttaatccaaaacctattcaatttaaattcggatatttctgattacgaacaacctcgtaatggcgaaaagtcaacaaatgattcgaataaaaacagtgacaatagcatcaaaaatgacactgatacgcaatcatctaaacaagataaagcagacaatcaaaaagcacctaaatcaaacaatacaaaaccaagtacatctaataagcaaccaaattcgccaaagccaacacaacctaatcaatcaaatagtcaaccagcaagtgacgataaagcaaatcaaaaatcttcatcgaaagataatcaatcaatgtcagattcggctttagactctattttggatcaatacagtgaagatgcaaagaaaacacaaaaagattatgcatctcaatctaaaaaagacaaaaatgaaaaatctaatacaaagaatccacagttaccaacacaagatgaattgaaacataaatctaaacctgctcaatcattcaataacgatgttaatcaaaaggatacacgtgcaacatcattattcgaaacagatcctagtatatctaacaatgatgatagcggacaatttaacgttgttgactcaaaagatacacgtcaatttgtcaaatcaattgctaaagatgcacatcgcattggtcaagataacgatatttatgcgtctgtcatgattgcccaagcaatcttagaatctgactcaggtcgtagtgctttagctaagtcaccaaaccataatttattcggtatcaaaggtgcttttgaagggaattctgttccttttaacacattacaggctgatggtaataaattgtatagtattaatgctggattccgaaaatatccaagcacgaaagaatcactaaaaaactactctgaccttattaaaaatggtattgatggcaatcgaacaatttataaaccaacatggaaatcggaagccgattcttataaagatgcaacatcacacttatctaaaacatatgctacagatccaaactatgctaagaaattaaacagtattattaaacactatcaattaactcagtttgacgatgaacgcatgccagatttagataaatatgaacgttctatcaaggattatgatgattcatcagatgaattcaaacctttccgtgaggtatctgatagtatgccatatccacatggtcaaagcacttggtacgtatataaccgtatgaaacaatttggtacatctatctcaggtgatttaggtgatgcacataattggaataatcgagctcaataccgtgattatcaagtaagtcatacaccaaaacgtcatgctgctgttgtatttgaggctggacaatttggtgcagatcaacattacggtcatgtagcatttgttgaaaaagttaacagtgatggttctatcgttatttcagaatccaatgttaaaggattaggtatcatttctcatagaactatcaatgcagctgccgctgaagaattatcatatattacaggtaaa

SEQ ID NO: 42 - protein, 390 aa, Staphylococcus aureus
KVAKQGQYKNQDPIVLVHGFNGFTDDINPSVLAHYWGGNKMNIRQDLEENGYKAYEASISAFGSNYDRAVELYYYIKGGRVDYGAAHAAKYGHERYGKTYEGIYKDWKPGQKVHLVGHSMGGQTIRQLEELLRNGSREEIEYQKKHGGEISPLFKGNNDNMISSITTLGTPHNGTHASDLAGNEALVRQIVFDIGKMFGNKNSRVDFGLAQWGLKQKPNESYIDYVKRVKQSNLWKSKDNGFYDLTREGATDLNRKTSLNPNIVYKTYTGEATHKALNSDRQKADLNMFFPFVITGNLIGKATEKEWRENDGLVSVISSQHPFNQAYTNATDKIQKGIWQVTPTKHDWDHVDFVGQDSSDTVRTREELQDFWHHLADDLVKTEKVTDTKQ

SEQ ID NO: 43 - DNA, 1170 bp, Staphylococcus aureus
aaagttgccaaacaagggcagtataaaaatcaagaccctatcgtgttagtgcatggtttcaatggatttacagatgatattaatccttcagtgttagctcattattggggcggtaataaaatgaacattcgccaagatttagaagaaaatggttacaaagcttatgaagcaagtataagtgcttttggaagtaactatgaccgcgcagttgaactttattattatatcaaaggcggtcgtgtagattatggtgcagcacatgcagcaaaatatggacatgaacgttatggaaaaacatacgaaggaatttacaaagactggaaaccaggacagaaggtacacctagttggacatagtatgggtggtcaaacgatacgtcaactagaagaattactgcgtaatggtagtcgtgaagaaatagagtatcaaaagaaacatggtggcgaaatttctccactattcaaaggtaataatgacaatatgatttcatcaattactactttaggaacgccacataatggaacgcatgcttcagatttagctggtaatgaagctttagtgagacaaattgtatttgatatcggtaaaatgtttggtaataaaaactctagagtagacttcgggttggctcaatggggtctaaaacagaagccaaatgaatcatacattgattatgtcaaacgcgttaaacaatctaatttatggaaatcaaaagataatggattttacgatctgacgcgtgagggtgcaacagatttaaatcgtaaaacgtcgttgaaccctaacattgtgtataaaacatacactggtgaagcaacgcacaaagcattaaatagcgatagacaaaaagcagacttaaatatgtttttcccatttgtgattactggtaacttaatcggtaaagctactgaaaaagaatggcgagaaaacgatggtttagtatccgttatttcttctcagcatccatttaatcaagcttatacaaatgcgacggataaaattcaaaaaggcatttggcaagtaacgcctacaaaacatgattgggatcatgttgattttgtcggacaagatagttctgatacagtgcgcacaagagaagaattacaagatttttggcatcatttagcagacgatttagtgaaaactgaaaaggtgactgatactaagcaa

SEQ ID NO: 44 - protein, 769 aa, Staphylococcus aureus
MDIGKKHVIPKSQYRRKRREFFHNEDREENLNQHQDKQNIDNTTSKKADKQIHKDSIDKHERFKNSLSSHLEQRNRDVNENKAEESKSNQDSKSAYNRDHYLTDDVSKKQNSLDSVDQDTEKSKYYEQNSEATLSTKSTDKVESTEMRKLSSDKNKVGHEEQHVLSKPSEHDKETRIDSESSRTDSDSSMQTEKIKKDSSDGNKSSNLKSEVISDKSNTVPKLSESDDEVNNQKPLTLPEEQKLKRQQSQNEQTKTYTYGDSEQNDKSNHENDLSHHIPSISDDKDNVMRENHIVDDNPDNDINTPSLSKTDDDRKLDEKIHVEDKHKQNADSSETVGYQSQSTASHRSTEKRNISINDHDKLNGQKTNTKTSANNNQKKATSKLNKGRATNNNYSDILKKFWMMYWPKLVILMGIIILIVILNAIFNNVNKNDRMNDNNDADAQKYTTTMKNANNTVKSVVTVENETSKDSSLPKDKASQDEVGSGVVYKKSGDTLYIVTNAHVVGDKENQKITFSNNKSVVGKVLGKDKWSDLAVVKATSSDSSVKEIAIGDSNNLVLGEPILVVGNPLGVDFKGTVTEGIISGLNRNVPIDFDKDNKYDMLMKAFQIDASVNPGNSGGAVVNREGKLIGVVAAKISMPNVENMSFAIPVNEVQKIVKDLETKGKIDYPDVGVKMKNIASLNSFERQAVKLPGKVKNGVVVDQVDNNGLADQSGLKKGDVITELDGKLLEDDLRFRQIIFSHKDDLKSITAKIYRDGKEKEINIKLK

SEQ ID NO: 45 - protein, 342 aa, Artificial sequence
  Mutated sequence
  VARIANT (192)..(192)
NNVNKNDRMNDNNDADAQKYTTTMKNANNTVKSVVTVENETSKDSSLPKDKASQDEVGSGVVYKKSGDTLYIVTNAHVVGDKENQKITFSNNKSVVGKVLGKDKWSDLAVVKATSSDSSVKEIAIGDSNNLVLGEPILVVGNPLGVDFKGTVTEGIISGLNRNVPIDFDKDNKYDMLMKAFQIDASVNPGNAGGAVVNREGKLIGVVAAKISMPNVENMSFAIPVNEVQKIVKDLETKGKIDYPDVGVKMKNIASLNSFERQAVKLPGKVKNGVVVDQVDNNGLADQSGLKKGDVITELDGKLLEDDLRFRQIIFSHKDDLKSITAKIYRDGKEKEINIKLK

SEQ ID NO: 46 - protein, 763 aa, Artificial sequence
  Mutated sequence
  VARIANT (192)..(192)
  MISC_FEATURE (343)..(354): Linker
NNVNKNDRMNDNNDADAQKYTTTMKNANNTVKSVVTVENETSKDSSLPKDKASQDEVGSGVVYKKSGDTLYIVTNAHVVGDKENQKITFSNNKSVVGKVLGKDKWSDLAVVKATSSDSSVKEIAIGDSNNLVLGEPILVVGNPLGVDFKGTVTEGIISGLNRNVPIDFDKDNKYDMLMKAFQIDASVNPGNAGGAVVNREGKLIGVVAAKISMPNVENMSFAIPVNEVQKIVKDLETKGKIDYPDVGVKMKNIASLNSFERQAVKLPGKVKNGVVVDQVDNNGLADQSGLKKGDVITELDGKLLEDDLRFRQIIFSHKDDLKSITAKIYRDGKEKEINIKLKGSGGGAGSGGGAMDIGKKHVIPKSQYRRKRREFFHNEDREENLNQHQDKQNIDNTTSKKADKQIHKDSIDKHERFKNSLSSHLEQRNRDVNENKAEESKSNQDSKSAYNRDHYLTDDVSKKQNSLDSVDQDTEKSKYYEQNSEATLSTKSTDKVESTEMRKLSSDKNKVGHEEQHVLSKPSEHDKETRIDSESSRTDSDSSMQTEKIKKDSSDGNKSSNLKSEVISDKSNTVPKLSESDDEVNNQKPLTLPEEQKLKRQQSQNEQTKTYTYGDSEQNDKSNHENDLSHHIPSISDDKDNVMRENHIVDDNPDNDINTPSLSKTDDDRKLDEKIHVEDKHKQNADSSETVGYQSQSTASHRSTEKRNISINDHDKLNGQKTNTKTSANNNQKKATSKLNKGRATNNNYSDILKKFWMMYWPK

SEQ ID NO: 47 - protein, 1009 aa, Staphylococcus aureus
MKKKNWIYALIVTLIIIIAIVSMIFFVQTKYGDQSEKGSQSVSNKNNKIHIAIVNEDQPTTYNGKKVELGQAFIKRLANEKNYKFETVTRNVAESGLKNGGYQVMIVIPENFSKLAMQLDAKTPSKISLQYKTAVGQKEEVAKNTEKVVSNVLNDFNKNLVEIYLTSIIDNLHNAQKNVGAIMTREHGVNSKFSNYLLNPINDFPELFTDTLVNSISANKDITKWFQTYNKSLLSANSDTFRVNTDYNVSTLIEKQNSLFDEHNTAMDKMLQDYKSQKDSVELDNYINALKQMDSQIDQQSSMQDTGKEEYKQTVKENLDKLREIIQSQESPFSKGMIEDYRKQLTESLQDELANNKDLQDALNSIKMNNAQFAENLEKQLHDDIVKEPDSDTTFIYNMSKQDFIAAGLNEDEANKYEAIVKEAKRYKNEYNLKKPLAEHINLTDYDNQVAQDTSSLINDGVKVQRTETIKSNDINQLTVATDPHFNFEGDIKINGKKYDIKDQSVQLDTSNKEYKVEVNGVAKLKKDAEKDFLKDKTMHLQLLFGQANRQDEPNDKKATSVVDVTLNHNLDGRLSKDALSQQLSALSRFDAHYKMYTDTKGREDKPFDNKRLIDMMVDQVINDMESFKDDKVAVLHQIDSMEENSDKLIDDILNNKKNTTKNKEDISKLIDQLENVKKTFAEEPQEPKIDKGKNDEFNTMSSNLDKEISRISEKSTQLLSDTQESKTIADSVSGQLNQLDNNVNKLHATGRALGVRANDLNRQMAKNDKDNELFAKEFKKVLQNSKDGDRQNQALKAFMSNPVQKKNLENVLANNGNTDVISPTLFVLLMYLLSMITAYIFYSYERAKGQMNFIKDDYSSKNNLWNNAITSGVIGATGLVEGLIVGLIAMNKFHVLAGYRAKFILMVILTMMVFVLINTYLLRQVKSIGMFLMIAALGLYFVAMNNLKAAGQGVTNKISPLSYIDNMFFNYLNAEHPIGLALVILTVLVIIGFVLNMFIKHFKKERLI

SEQ ID NO: 48 - protein, 793 aa, Staphylococcus aureus
QTKYGDQSEKGSQSVSNKNNKIHIAIVNEDQPTTYNGKKVELGQAFIKRLANEKNYKFETVTRNVAESGLKNGGYQVMIVIPENFSKLAMQLDAKTPSKISLQYKTAVGQKEEVAKNTEKVVSNVLNDFNKNLVEIYLTSIIDNLHNAQKNVGAIMTREHGVNSKFSNYLLNPINDFPELFTDTLVNSISANKDITKWFQTYNKSLLSANSDTFRVNTDYNVSTLIEKQNSLFDEHNTAMDKMLQDYKSQKDSVELDNYINALKQMDSQIDQQSSMQDTGKEEYKQTVKENLDKLREIIQSQESPFSKGMIEDYRKQLTESLQDELANNKDLQDALNSIKMNNAQFAENLEKQLHDDIVKEPDSDTTFIYNMSKQDFIAAGLNEDEANKYEAIVKEAKRYKNEYNLKKPLAEHINLTDYDNQVAQDTSSLINDGVKVQRTETIKSNDINQLTVATDPHFNFEGDIKINGKKYDIKDQSVQLDTSNKEYKVEVNGVAKLKKDAEKDFLKDKTMHLQLLFGQANRQDEPNDKKATSVVDVTLNHNLDGRLSKDALSQQLSALSRFDAHYKMYTDTKGREDKPFDNKRLIDMMVDQVINDMESFKDDKVAVLHQIDSMEENSDKLIDDILNNKKNTTKNKEDISKLIDQLENVKKTFAEEPQEPKIDKGKNDEFNTMSSNLDKEISRISEKSTQLLSDTQESKTIADSVSGQLNQLDNNVNKLHATGRALGVRANDLNRQMAKNDKDNELFAKEFKKVLQNSKDGDRQNQALKAFMSNPVQKKNLENVLANNGNTD

SEQ ID NO: 49 - protein, 30 aa, Staphylococcus aureus
KAAGQGVTNKISPLSYIDNMFFNYLNAEHP

SEQ ID NO: 50 - protein, 6 aa, Artificial sequence
  Linker
GSGGGA

SEQ ID NO: 51 - protein, 12 aa, Artificial sequence
  Linker
GSGGGAGSGGGA

SEQ ID NO: 52 - protein, 12 aa, Artificial sequence
  Linker
KPEPKPAPAPKP

SEQ ID NO: 53 - DNA, 18 bp, Artificial sequence
  DNA encoding linker
ggttctggcggaggggct

SEQ ID NO: 54 - DNA, 36 bp, Artificial sequence
  Linker encoding DNA
ggttctggcggaggggctggaagtggtgggggcgcc

SEQ ID NO: 55 - DNA, 36 bp, Artificial sequence
  Linker encoding DNA
aaacctgagccgaaaccagctcccgcccctaagcca

SEQ ID NO: 56 - protein, 582 aa, Artificial sequence
  Fusion protein
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHNGSGGGASETEVSVRSASSDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 57 - protein, 779 aa, Artificial sequence
  Fusion protien
IDSKNKPANSDIKFEVTQKSDAVKALKELPKSENVKNIYQDYAVTDVKTDKKGFTHYTLQPSVDGVHAPDKEVKVHADKSGKVVLINGDTDAKKVKPTNKVTLSKDDAADKAFKAVKIDKNKAKNLKDKVIKENKVEIDGDSNKYVYNVELITVTPEISHWKVKIDAQTGEILEKMNLVKEAAETGKGKGVLGDTKDININSIDGGFSLEDLTHQGKLSAFSFNDQTGQATLITNEDENFVKDEQRAGVDANYYAKQTYDYYKDTFGRESYDNQGSPIVSLTHVNNYGGQDNRNNAAWIGDKMIYGDGDGRTFTSLSGANDVVAHALTHGVTQETANLEYKDQSGALNESFSDVFGYFVDDEDFLMGEDVYTPGKEGDALRSMSNPEQFGQPAHMKDYVFTEKDNGGVHTNSGIPNKAAYNVIQAIGKSKSEQIYYRALTEYLTSNSNFKDSKDALYQAAKDLYDEQTAEQVYEAWNEVGVEGSGGGAAQVDEAQKNAFYQVLNMPNLNADQRNGFIQSLKDAPAKSANVLGEAQKLNDSQAPKADAQQNNVNKDQKSAFYEILNMPNLNEAQRNGFIQSLKDAPAKSTNVLGEAKKLNESQAPKADNNVNKEQKNAFYEILNMPNLNEEQRNGFIQSLKDAPAKSANLLSEAKKLNESQAPKADNKVNKEQKNAFYEILHLPNLNEEQRNGFIQSLKDAPAKSANLLAEAKKLNDAQAPKADNKVNKEQKNAFYEILHLPNLTEEQRNGFIQSLKDAPAKSKEILAEAKKLNDAQAPK

SEQ ID NO: 58 - protein, 706 aa, Artificial sequence
  Fusion protein
MDIGKKHVIPKSQYRRKRREFFHNEDREENLNQHQDKQNIDNTTSKKADKQIHKDSIDKHERFKNSLSSHLEQRNRDVNENKAEESKSNQDSKSAYNRDHYLTDDVSKKQNSLDSVDQDTEKSKYYEQNSEATLSTKSTDKVESTEMRKLSSDKNKVGHEEQHVLSKPSEHDKETRIDSESSRTDSDSSMQTEKIKKDSSDGNKSSNLKSEVISDKSNTVPKLSESDDEVNNQKPLTLPEEQKLKRQQSQNEQTKTYTYGDSEQNDKSNHENDLSHHIPSISDDKDNVMRENHIVDDNPDNDINTPSLSKTDDDRKLDEKIHVEDKHKQNADSSETVGYQSQSTASHRSTEKRNISINDHDKLNGQKTNTKTSANNNQKKATSKLNKGRATNNNYSDILKKFWMMYWPKGSGGGAAQVDEAQKNAFYQVLNMPNLNADQRNGFIQSLKDAPAKSANVLGEAQKLNDSQAPKADAQQNNVNKDQKSAFYEILNMPNLNEAQRNGFIQSLKDAPAKSTNVLGEAKKLNESQAPKADNNVNKEQKNAFYEILNMPNLNEEQRNGFIQSLKDAPAKSANLLSEAKKLNESQAPKADNKVNKEQKNAFYEILHLPNLNEEQRNGFIQSLKDAPAKSANLLAEAKKLNDAQAPKADNKVNKEQKNAFYEILHLPNLTEEQRNGFIQSLKDAPAKSKEILAEAKKLNDAQAPK

SEQ ID NO: 59 - protein, 988 aa, Artificial sequence
  Fusion protein
KVAKQGQYKNQDPIVLVHGFNGFTDDINPSVLAHYWGGNKMNIRQDLEENGYKAYEASISAFGSNYDRAVELYYYIKGGRVDYGAAHAAKYGHERYGKTYEGIYKDWKPGQKVHLVGHSMGGQTIRQLEELLRNGSREEIEYQKKHGGEISPLFKGNNDNMISSITTLGTPHNGTHASDLAGNEALVRQIVFDIGKMFGNKNSRVDFGLAQWGLKQKPNESYIDYVKRVKQSNLWKSKDNGFYDLTREGATDLNRKTSLNPNIVYKTYTGEATHKALNSDRQKADLNMFFPFVITGNLIGKATEKEWRENDGLVSVISSQHPFNQAYTNATDKIQKGIWQVTPTKHDWDHVDFVGQDSSDTVRTREELQDFWHHLADDLVKTEKVTDTKQGSGGGADTPQKDTTAKTTSHDSKKSNDDETSKDTTSKDIDKADNNNTSNQDNNDKKFKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKANQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLQADGNKLYSINAGFRKYPSTKESLKNYSDLIKNGIDGNRTIYKPTWKSEADSYKDATSHLSKTYATDPNYAKKLNSIIKHYQLTQFDDERMPDLDKYERSIKDYDDSSDEFKPFREVSDSMPYPHGQSTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGK

SEQ ID NO: 60 - protein, 1189 aa, Artificial sequence
  Fusion protein
QTKYGDQSEKGSQSVSNKNNKIHIAIVNEDQPTTYNGKKVELGQAFIKRLANEKNYKFETVTRNVAESGLKNGGYQVMIVIPENFSKLAMQLDAKTPSKISLQYKTAVGQKEEVAKNTEKVVSNVLNDFNKNLVEIYLTSIIDNLHNAQKNVGAIMTREHGVNSKFSNYLLNPINDFPELFTDTLVNSISANKDITKWFQTYNKSLLSANSDTFRVNTDYNVSTLIEKQNSLFDEHNTAMDKMLQDYKSQKDSVELDNYINALKQMDSQIDQQSSMQDTGKEEYKQTVKENLDKLREIIQSQESPFSKGMIEDYRKQLTESLQDELANNKDLQDALNSIKMNNAQFAENLEKQLHDDIVKEPDSDTTFIYNMSKQDFIAAGLNEDEANKYEAIVKEAKRYKNEYNLKKPLAEHINLTDYDNQVAQDTSSLINDGVKVQRTETIKSNDINQLTVATDPHFNFEGDIKINGKKYDIKDQSVQLDTSNKEYKVEVNGVAKLKKDAEKDFLKDKTMHLQLLFGQANRQDEPNDKKATSVVDVTLNHNLDGRLSKDALSQQLSALSRFDAHYKMYTDTKGREDKPFDNKRLIDMMVDQVINDMESFKDDKVAVLHQIDSMEENSDKLIDDILNNKKNTTKNKEDISKLIDQLENVKKTFAEEPQEPKIDKGKNDEFNTMSSNLDKEISRISEKSTQLLSDTQESKTIADSVSGQLNQLDNNVNKLHATGRALGVRANDLNRQMAKNDKDNELFAKEFKKVLQNSKDGDRQNQALKAFMSNPVQKKNLENVLANNGNTDGSGGGAKVAKQGQYKNQDPIVLVHGFNGFTDDINPSVLAHYWGGNKMNIRQDLEENGYKAYEASISAFGSNYDRAVELYYYIKGGRVDYGAAHAAKYGHERYGKTYEGIYKDWKPGQKVHLVGHSMGGQTIRQLEELLRNGSREEIEYQKKHGGEISPLFKGNNDNMISSITTLGTPHNGTHASDLAGNEALVRQIVFDIGKMFGNKNSRVDFGLAQWGLKQKPNESYIDYVKRVKQSNLWKSKDNGFYDLTREGATDLNRKTSLNPNIVYKTYTGEATHKALNSDRQKADLNMFFPFVITGNLIGKATEKEWRENDGLVSVISSQHPFNQAYTNATDKIQKGIWQVTPTKHDWDHVDFVGQDSSDTVRTREELQDFWHHLADDLVKTEKVTDTKQ

SEQ ID NO: 61 - protein, 805 aa, Artificial sequence
  Fusion protein
MDIGKKHVIPKSQYRRKRREFFHNEDREENLNQHQDKQNIDNTTSKKADKQIHKDSIDKHERFKNSLSSHLEQRNRDVNENKAEESKSNQDSKSAYNRDHYLTDDVSKKQNSLDSVDQDTEKSKYYEQNSEATLSTKSTDKVESTEMRKLSSDKNKVGHEEQHVLSKPSEHDKETRIDSESSRTDSDSSMQTEKIKKDSSDGNKSSNLKSEVISDKSNTVPKLSESDDEVNNQKPLTLPEEQKLKRQQSQNEQTKTYTYGDSEQNDKSNHENDLSHHIPSISDDKDNVMRENHIVDDNPDNDINTPSLSKTDDDRKLDEKIHVEDKHKQNADSSETVGYQSQSTASHRSTEKRNISINDHDKLNGQKTNTKTSANNNQKKATSKLNKGRATNNNYSDILKKFWMMYWPKGSGGGAKVAKQGQYKNQDPIVLVHGFNGFTDDINPSVLAHYWGGNKMNIRQDLEENGYKAYEASISAFGSNYDRAVELYYYIKGGRVDYGAAHAAKYGHERYGKTYEGIYKDWKPGQKVHLVGHSMGGQTIRQLEELLRNGSREEIEYQKKHGGEISPLFKGNNDNMISSITTLGTPHNGTHASDLAGNEALVRQIVFDIGKMFGNKNSRVDFGLAQWGLKQKPNESYIDYVKRVKQSNLWKSKDNGFYDLTREGATDLNRKTSLNPNIVYKTYTGEATHKALNSDRQKADLNMFFPFVITGNLIGKATEKEWRENDGLVSVISSQHPFNQAYTNATDKIQKGIWQVTPTKHDWDHVDFVGQDSSDTVRTREELQDFWHHLADDLVKTEKVTDTKQ

SEQ ID NO: 62 - protein, 879 aa, Artificial sequence
  Fusion protein
DTPQKDTTAKTTSHDSKKSNDDETSKDTTSKDIDKADNNNTSNQDNNDKKFKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKANQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLQADGNKLYSINAGFRKYPSTKESLKNYSDLIKNGIDGNRTIYKPTWKSEADSYKDATSHLSKTYATDPNYAKKLNSIIKHYQLTQFDDERMPDLDKYERSIKDYDDSSDEFKPFREVSDSMPYPHGQSTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGKGSGGGADIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 63 - protein, 771 aa, Artificial sequence
  Fusion protien
IDSKNKPANSDIKFEVTQKSDAVKALKELPKSENVKNIYQDYAVTDVKTDKKGFTHYTLQPSVDGVHAPDKEVKVHADKSGKVVLINGDTDAKKVKPTNKVTLSKDDAADKAFKAVKIDKNKAKNLKDKVIKENKVEIDGDSNKYVYNVELITVTPEISHWKVKIDAQTGEILEKMNLVKEAAETGKGKGVLGDTKDININSIDGGFSLEDLTHQGKLSAFSFNDQTGQATLITNEDENFVKDEQRAGVDANYYAKQTYDYYKDTFGRESYDNQGSPIVSLTHVNNYGGQDNRNNAAWIGDKMIYGDGDGRTFTSLSGANDVVAHALTHGVTQETANLEYKDQSGALNESFSDVFGYFVDDEDFLMGEDVYTPGKEGDALRSMSNPEQFGQPAHMKDYVFTEKDNGGVHTNSGIPNKAAYNVIQAIGKSKSEQIYYRALTEYLTSNSNFKDSKDALYQAAKDLYDEQTAEQVYEAWNEVGVEGSGGGANTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 64 - protein, 570 aa, Artificial sequence
  Fusion protein
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHNGSGGGADIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 65 - protein, 779 aa, Artificial sequence
  Fusion protein
IDSKNKPANSDIKFEVTQKSDAVKALKELPKSENVKNIYQDYAVTDVKTDKKGFTHYTLQPSVDGVHAPDKEVKVHADKSGKVVLINGDTDAKKVKPTNKVTLSKDDAADKAFKAVKIDKNKAKNLKDKVIKENKVEIDGDSNKYVYNVELITVTPEISHWKVKIDAQTGEILEKMNLVKEAAETGKGKGVLGDTKDININSIDGGFSLEDLTHQGKLSAFSFNDQTGQATLITNEDENFVKDEQRAGVDANYYAKQTYDYYKDTFGRESYDNQGSPIVSLTHVNNYGGQDNRNNAAWIGDKMIYGDGDGRTFTSLSGANDVVAHALTHGVTQETANLEYKDQSGALNESFSDVFGYFVDDEDFLMGEDVYTPGKEGDALRSMSNPEQFGQPAHMKDYVFTEKDNGGVHTNSGIPNKAAYNVIQAIGKSKSEQIYYRALTEYLTSNSNFKDSKDALYQAAKDLYDEQTAEQVYEAWNEVGVEGSGGGAAQVDEAQKNAFYQVLNMPNLNADQRNGFIQSLKDAPSQSAKVLGEAQKLNDSQAPKADAQQNNVNKDQKSAFYEILNMPNLNEAQRNGFIQSLKDAPSQSTKVLGEAKKLNESQAPKADNNVNKEQKNAFYEILNMPNLNEEQRNGFIQSLKDAPSQSAKLLSEAKKLNESQAPKADNKVNKEQKNAFYEILHLPNLNEEQRNGFIQSLKDAPSQSAKLLAEAKKLNDAQAPKADNKVNKEQKNAFYEILHLPNLTEEQRNGFIQSLKDAPSVSKKILAEAKKLNDAQAPK

SEQ ID NO: 66 - protein, 1082 aa, Artificial sequence
  Fusion protein
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHNGSGGGAQTKYGDQSEKGSQSVSNKNNKIHIAIVNEDQPTTYNGKKVELGQAFIKRLANEKNYKFETVTRNVAESGLKNGGYQVMIVIPENFSKLAMQLDAKTPSKISLQYKTAVGQKEEVAKNTEKVVSNVLNDFNKNLVEIYLTSIIDNLHNAQKNVGAIMTREHGVNSKFSNYLLNPINDFPELFTDTLVNSISANKDITKWFQTYNKSLLSANSDTFRVNTDYNVSTLIEKQNSLFDEHNTAMDKMLQDYKSQKDSVELDNYINALKQMDSQIDQQSSMQDTGKEEYKQTVKENLDKLREIIQSQESPFSKGMIEDYRKQLTESLQDELANNKDLQDALNSIKMNNAQFAENLEKQLHDDIVKEPDSDTTFIYNMSKQDFIAAGLNEDEANKYEAIVKEAKRYKNEYNLKKPLAEHINLTDYDNQVAQDTSSLINDGVKVQRTETIKSNDINQLTVATDPHFNFEGDIKINGKKYDIKDQSVQLDTSNKEYKVEVNGVAKLKKDAEKDFLKDKTMHLQLLFGQANRQDEPNDKKATSVVDVTLNHNLDGRLSKDALSQQLSALSRFDAHYKMYTDTKGREDKPFDNKRLIDMMVDQVINDMESFKDDKVAVLHQIDSMEENSDKLIDDILNNKKNTTKNKEDISKLIDQLENVKKTFAEEPQEPKIDKGKNDEFNTMSSNLDKEISRISEKSTQLLSDTQESKTIADSVSGQLNQLDNNVNKLHATGRALGVRANDLNRQMAKNDKDNELFAKEFKKVLQNSKDGDRQNQALKAFMSNPVQKKNLENVLANNGNTD

SEQ ID NO: 67 - protein, 696 aa, Artificial sequence
  Fusion protein
MDIGKKHVIPKSQYRRKRREFFHNEDREENLNQHQDKQNIDNTTSKKADKQIHKDSIDKHERFKNSLSSHLEQRNRDVNENKAEESKSNQDSKSAYNRDHYLTDDVSKKQNSLDSVDQDTEKSKYYEQNSEATLSTKSTDKVESTEMRKLSSDKNKVGHEEQHVLSKPSEHDKETRIDSESSRTDSDSSMQTEKIKKDSSDGNKSSNLKSEVISDKSNTVPKLSESDDEVNNQKPLTLPEEQKLKRQQSQNEQTKTYTYGDSEQNDKSNHENDLSHHIPSISDDKDNVMRENHIVDDNPDNDINTPSLSKTDDDRKLDEKIHVEDKHKQNADSSETVGYQSQSTASHRSTEKRNISINDHDKLNGQKTNTKTSANNNQKKATSKLNKGRATNNNYSDILKKFWMMYWPKGSGGGADIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 68 - protein, 763 aa, Artificial sequence; identical to SEQ ID NO: 46
  Fusion protein

SEQ ID NO: 69 - DNA, 1746 bp, Artificial sequence
  Fuson protein encoding DNA
aacaccaacatcgagaacattggcgatggtgcggaagtgattaagcgtaccgaagacgttagcagcaagaaatggggcgtgacccagaacgttcaattcgacttcgtgaaggataagaagtacaacaaggatgcgctgatcgttaagatgcagggtttcattaacagccgtaccagctttagcgacgttaaaggcagcggttacgagctgaccaagcgtatgatctggccgttccaatataacattggcctgaccaccaaagatccgaacgtgagcctgatcaactacctgccgaagaacaaaattgaaaccaccgacgttggccagaccctgggttataacatcggtggcaacttccaaagcgcgccgagcattggtggcaacggtagctttaactacagcaaaaccatcagctacacccagaagagctatgtgagcgaggttgataaacaaaacagcaagagcgtgaaatggggcgttaaggcgaacgaattcgtgaccccggacggtaagaaaagcgcgcacgatcgttacctgtttgttcaaagcccgaacggtccgaccggtagcgcgcgtgagtattttgcgccggacaaccagctgccgccgctggtgcaaagcggcttcaacccgagctttattaccaccctgagccacgaaaagggcagcagcgataccagcgagtttgaaatcagctacggtcgtaacctggacattacctatgcgaccctgttcccgcgtaccggtatctacgcggagcgtaaacacaacgcgttcgttaaccgtaactttgtggttcgttatgaggtgaactggaagacccacgaaatcaaggttaaaggtcataacggtagcggtggcggtgcgagcgaaaccgaagtgagcgtgcgcagcgcgagcagcgacattggcagcaacaccaccgtgaaaaccggtgacctggttacctacgataaggaaaacggtatgctgaagaaagtgttctatagctttatcgacgataaaaaccacaacaagaaactgctggtgatccgtaccaagggcaccattgcgggccagtaccgtgtttatagcgaggaaggcgcgaacaaaagcggtctggcgtggccgagcgcgtttaaggtgcagctgcaactgccggacaacgaggttgcgcaaatcagcgattactatccgcgtaacagcattgacaccaaagaatacatgagcaccctgacctatggcttcaacggtaacgttaccggcgacgataccggcaagatcggcggtctgattggcgcgaacgtgagcatcggtcacaccctgaaatacgttcagccggattttaagaccattctggagagcccgaccgacaagaaagtgggctggaaagttatcttcaacaacatggtgaaccagaactggggtccgtacgaccgtgatagctggaacccggtttatggcaaccaactgttcatgaaaacccgtaacggtagcatgaaggcggcggacaactttctggatccgaacaaggcgagcagcctgctgagcagcggtttcagcccggattttgcgaccgtgatcaccatggaccgtaaggcgagcaaacagcaaaccaacatcgatgtgatttacgaacgtgttcgtgacgattatcaactgcactggaccagcaccaactggaaaggtaccaacaccaaggacaaatggatcgatcgtagcagcgagcgttacaagattgactgggaaaaagaggaaatgaccaac

SEQ ID NO: 70 - DNA, 2337 bp, Artificial sequence
  Fusion protein encoding DNA
atcgacagcaagaacaaaccggcgaacagcgatattaagttcgaagtgacccagaaaagcgatgcggttaaagcgctgaaggaactgccgaagagcgagaacgtgaaaaacatctaccaggactatgcggtgaccgacgttaaaaccgataagaaaggttttacccactacaccctgcagccgagcgtggacggtgttcatgcgccggataaagaggtgaaggttcacgcggacaaaagcggcaaggtggttctgatcaacggtgacaccgatgcgaagaaagtgaagccgaccaacaaagttaccctgagcaaggacgatgcggcggacaaagcgttcaaggcggttaaaattgataagaacaaagcgaagaacctgaaagacaaggtgatcaaggagaacaaagttgaaattgacggtgatagcaacaaatacgtgtataacgttgaactgatcaccgtgaccccggagattagccactggaaagttaagatcgatgcgcagaccggcgagattctggaaaagatgaacctggtgaaagaggcggcggaaaccggcaaaggcaagggcgttctgggcgacaccaaggatatcaacattaacagcatcgacggtggctttagcctggaagatctgacccaccagggtaaactgagcgcgttcagctttaacgaccagaccggccaagcgaccctgattaccaacgaggacgaaaacttcgtgaaagatgaacagcgtgcgggtgttgatgcgaactactatgcgaagcaaacctacgactactataaagatacctttggccgtgagagctatgacaaccagggtagcccgatcgtgagcctgacccacgttaacaactacggtggccaagataaccgtaacaacgcggcgtggatcggcgacaagatgatttatggtgacggcgatggtcgtaccttcaccagcctgagcggtgcgaacgatgttgttgcgcatgcgctgacccatggtgtgacccaggaaaccgcgaacctggagtacaaggatcaaagcggcgcgctgaacgaaagcttcagcgacgtgttcggttactttgttgacgatgaagactttctgatgggcgaggatgtttataccccgggcaaagaaggtgacgcgctgcgtagcatgagcaacccggagcagttcggtcaaccggcgcacatgaaggactacgtgtttaccgagaaagataacggtggcgttcacaccaacagcggtatcccgaacaaggcggcgtataacgtgatccaggcgattggcaaaagcaagagcgaacaaatttactatcgtgcgctgaccgagtacctgaccagcaacagcaacttcaaggacagcaaagatgcgctgtaccaggcggcgaaggacctgtatgatgagcagaccgcggaacaagtttatgaggcgtggaacgaagtgggcgttgagggtagcggtggcggtgcggcgcaggtggacgaagcgcagaagaacgcgttctaccaagttctgaacatgccgaacctgaacgcggatcagcgtaacggttttatccaaagcctgaaggatgcgccggcgaaaagcgcgaacgtgctgggtgaagcgcagaagctgaacgacagccaagcgccgaaagcggatgcgcagcaaaacaacgttaacaaggaccagaaaagcgcgttctatgaaatcctgaatatgccgaatctgaacgaggcgcagcgtaacggttttattcagagcctgaaagacgcgccggcgaaaagcaccaacgtgctgggcgaagcgaagaaactgaacgagagccaggcgccgaaggcggacaacaacgttaacaaggaacagaagaacgcgttttatgagatcctgaatatgccgaacctgaacgaggaacaacgcaacggttttattcaatctttaaaagacgcgccggcgaagagcgcgaacctgctgagcgaggcgaagaaactgaatgagagccaggcgccgaaagcggacaacaaggtgaacaaagaacaaaagaacgcgttctatgagatcctgcacctgccgaatctgaatgaagaacagcgtaacggctttattcagtctttaaaagatgctcctgctaagtctgcgaacctgctggcggaggcgaagaaactgaacgacgcgcaggcgccgaaggcggataacaaagtgaataaagagcagaagaacgctttttatgagattctgcacctgccgaacctgaccgaggaacaacgtaatggcttcattcagagcctgaaagatgcgccggcgaagagcaaggaaattctggcggaagcgaagaaactgaatgacgcgcaggcgccgaaa

SEQ ID NO: 71 - DNA, 2118 bp, Artificial sequence
  Fusion protein encoding sequence
atggacatcggtaagaaacacgtgattccgaagagccagtaccgtcgtaaacgtcgtgagttctttcacaacgaagaccgtgaggaaaacctgaaccagcaccaagataagcagaacattgacaacaccaccagcaagaaagcggataagcaaatccacaaagatagcattgacaagcacgagcgtttcaaaaacagcctgagcagccacctggagcagcgtaaccgtgacgtgaacgaaaacaaggcggaggaaagcaaaagcaaccaagacagcaaaagcgcgtacaaccgtgatcactatctgaccgacgatgtgagcaagaaacagaacagcctggatagcgttgatcaagacaccgaaaagagcaaatactatgagcagaacagcgaagcgaccctgagcaccaagagcaccgacaaagttgagagcaccgaaatgcgtaagctgagcagcgataagaacaaagtgggccacgaggaacagcacgttctgagcaagccgagcgagcacgataaagaaacccgtatcgacagcgagagcagccgtaccgatagcgacagcagcatgcaaaccgaaaagattaagaaagatagcagcgacggtaacaagagcagcaacctgaaaagcgaggtgatcagcgacaagagcaacaccgttccgaaactgagcgagagcgacgatgaagttaacaaccagaaaccgctgaccctgccggaggaacaaaagctgaaacgtcagcaaagccagaacgagcaaaccaagacctacacctatggcgatagcgagcagaacgacaaaagcaaccacgaaaacgacctgagccaccacatcccgagcattagcgacgataaggataacgtgatgcgtgaaaaccacatcgttgacgataacccggataacgacattaacaccccgagcctgagcaaaaccgacgatgaccgtaagctggatgagaaaatccacgtggaagacaagcacaaacagaacgcggatagcagcgaaaccgttggctaccagagccaaagcaccgcgagccaccgtagcaccgaaaagcgtaacatcagcattaacgatcacgacaaactgaacggtcagaagaccaacaccaaaaccagcgcgaacaacaaccaaaagaaagcgaccagcaagctgaacaaaggccgtgcgaccaacaacaactacagcgacatcctgaagaaattctggatgatgtactggccgaagggtagcggtggcggtgcggcgcaggtggatgaggcgcagaagaacgcgttctatcaggttctgaacatgccgaacctgaacgcggaccagcgtaacggctttatccaaagcctgaaggatgcgccggcgaaaagcgcgaacgtgctgggtgaagcgcagaagctgaacgacagccaagcgccgaaagcggatgcgcagcaaaacaacgttaacaaggaccagaagagcgcgttctacgagatcctgaatatgccgaatctgaacgaagcgcagcgtaacggctttattcaaagcctgaaggacgcgccggcgaaaagcaccaatgtgctgggcgaggcgaagaaactgaacgaaagccaggcgccgaaagcggataacaacgttaacaaggaacagaagaacgcgttttatgaaatcctgaatatgccgaacctgaacgaggaacaacgcaatggtttcatccagagcctgaaagatgcgccggcgaagagcgcgaacctgctgagcgaagcgaagaaactgaatgaaagccaggcgccgaaggcggataacaaggtgaacaaagagcaaaagaacgcgttctacgaaatcctgcacctgccgaatctgaatgaggaacagcgtaacggtttcatccaatctttaaaagatgctcctgcgaaaagcgcgaacctgctggcggaagcgaagaaactgaacgatgcgcaagcgccgaaggcggacaacaaagttaacaaggagcagaagaacgcgttttacgagattctgcacctgccgaacctgaccgaggaacaacgcaacggtttcatccaaagtttaaaagatgctcccgcgaaaagcaaagagattctggcggaggcgaagaaactgaatgatgcgcaggcgccgaaa

SEQ ID NO: 72 - DNA, 2964 bp, Artificial sequence
  Fusion protein encoding DNA
aaagttgcgaagcagggccaatacaagaaccaggatccgatcgtgctggttcacggcttcaacggttttaccgacgatattaacccgagcgtgctggcgcactattggggtggcaacaagatgaacatccgtcaagacctggaggaaaacggttataaagcgtacgaggcgagcattagcgcgtttggcagcaactacgatcgtgcggttgaactgtactattacatcaagggtggccgtgtggactatggtgcggcgcatgcggcgaagtatggtcatgagcgttatggtaaaacctacgaaggcatctataaagactggaagccgggtcagaaagtgcacctggttggccacagcatgggtggccagaccattcgtcaactggaggaactgctgcgtaacggtagccgtgaggaaatcgagtaccaaaagaaacacggtggcgaaattagcccgctgttcaaaggcaacaacgataacatgatcagcagcattaccaccctgggtaccccgcacaacggtacccatgcgagcgacctggcgggtaacgaggcgctggttcgtcagatcgtgttcgatattggcaagatgtttggtaacaaaaacagccgtgttgacttcggtctggcgcagtggggcctgaagcaaaaaccgaacgaaagctatatcgattacgtgaaacgtgttaagcagagcaacctgtggaagagcaaagataacggtttttatgacctgacccgtgagggtgcgaccgacctgaaccgtaagaccagcctgaacccgaacattgtttataaaacctacaccggcgaagcgacccacaaggcgctgaacagcgatcgtcagaaagcggacctgaacatgttctttccgttcgtgatcaccggcaacctgattggtaaagcgaccgagaaggaatggcgtgagaacgacggtctggtgagcgttatcagcagccagcacccgtttaaccaagcgtacaccaacgcgaccgataaaatccagaagggtatttggcaagttaccccgaccaagcacgactgggatcacgtggacttcgttggccaggacagcagcgataccgtgcgtacccgtgaggaactgcaagatttttggcaccacctggcggacgatctggttaaaaccgagaaggtgaccgacaccaaacaaggtagcggtggcggtgcggataccccgcaaaaggacaccaccgcgaaaaccaccagccacgacagcaagaaaagcaacgacgatgaaaccagcaaagataccaccagcaaagacatcgataaggcggacaacaacaacaccagcaaccaggacaacaacgataagaaattcaagaccattgacgatagcaccagcgacagcaacaacatcatcgacttcatctacaagaacctgccgcagaccaacattaaccaactgctgaccaagaacaaatacgacgataactatagcctgaccaccctgatccagaacctgttcaacctgaacagcgacattagcgattatgagcaaccgcgtaacggcgagaagagcaccaacgatagcaacaagaacagcgacaacagcatcaaaaacgacaccgatacccagagcagcaaacaagacaaggcggataaccagaaagcgccgaagagcaataacaccaagccgagcaccagcaacaaacaaccgaacagcccgaagccgacccagccgaaccaaagcaacagccaaccggcgagcgacgataaagcgaaccagaagagcagcagcaaagacaaccaaagcatgagcgacagcgcgctggatagcatcctggaccagtacagcgaggatgcgaagaaaacccagaaggactatgcgagccaaagcaagaaagataaaaacgagaagagcaacaccaaaaacccgcagctgccgacccaagacgaactgaagcacaaaagcaagccggcgcagagcttcaacaacgatgttaaccaaaaggacacccgtgcgaccagcctgtttgaaaccgacccgagcatcagcaacaacgacgatagcggccagttcaacgtggttgacagcaaagatacccgtcaatttgttaagagcattgcgaaagatgcgcaccgtatcggtcaggacaacgatatttacgcgagcgtgatgatcgcgcaagcgattctggagagcgacagcggccgtagcgcgctggcgaagagcccgaaccacaacctgttcggcatcaagggtgcgtttgagggtaacagcgtgccgttcaacaccctgcaggcggatggtaacaagctgtacagcattaacgcgggctttcgtaaatatccgagcaccaaggagagcctgaaaaactatagcgatctgatcaagaacggcattgacggtaaccgtaccatctacaagccgacctggaaaagcgaagcggatagctataaggacgcgaccagccacctgagcaaaacctacgcgaccgacccgaactatgcgaagaaactgaacagcatcattaaacactaccagctgacccaattcgacgatgagcgtatgccggacctggataaatacgaacgtagcatcaaggattatgacgatagcagcgacgagttcaagccgtttcgtgaagttagcgatagcatgccgtatccgcacggtcagagcacctggtacgtttacaaccgtatgaaacaatttggcaccagcattagcggcgacctgggtgatgcgcacaactggaacaaccgtgcgcagtatcgtgactaccaagtgagccataccccgaagcgtcatgcggcggtggttttcgaagcgggccagtttggtgcggatcaacactacggtcacgtggcgttcgttgagaaggtgaacagcgacggcagcatcgttattagcgaaagcaacgtgaaaggcctgggtatcattagccatcgtaccatcaacgcggcggcggcggaggaactgagctatattaccggtaaa

SEQ ID NO: 73 - DNA, 3567 bp, Artificial sequence
  Fusion protein encoding DNA
caaacaaagtacggagatcaatcagaaaaggggtcacaaagtgtaagcaacaagaacaacaaaatccacattgcgatcgttaacgaggaccaaccgaccacctataacggtaagaaagtggaactgggccaggcgttcatcaagcgtctggcgaacgagaagaactacaaatttgaaaccgtgacccgtaacgttgcggagagcggtctgaagaacggtggctatcaagtgatgattgttatcccggaaaacttcagcaaactggcgatgcagctggacgcgaaaaccccgagcaagattagcctgcaatacaaaaccgcggtgggccagaaggaagaggtggcgaaaaacaccgagaaggtggttagcaacgtgctgaacgatttcaacaagaacctggttgaaatttacctgaccagcatcattgacaacctgcacaacgcgcagaagaacgtgggtgcgatcatgacccgtgagcacggcgttaacagcaaatttagcaactatctgctgaacccgattaacgatttcccggaactgtttaccgacaccctggttaacagcattagcgcgaacaaagatatcaccaagtggttccaaacctacaacaaaagcctgctgagcgcgaacagcgatacctttcgtgtgaacaccgactataacgttagcaccctgatcgagaagcagaacagcctgttcgacgaacacaacaccgcgatggataaaatgctgcaggactacaagagccaaaaagatagcgtggagctggacaactatattaacgcgctgaagcagatggacagccaaatcgatcagcaaagcagcatgcaagataccggtaaagaggaatacaagcagaccgttaaagagaacctggacaaactgcgtgagatcattcagagccaagaaagcccgttcagcaaaggcatgatcgaagattatcgtaagcaactgaccgagagcctgcaggacgaactggcgaacaacaaagacctgcaggatgcgctgaacagcatcaagatgaacaacgcgcaatttgcggagaacctggaaaaacagctgcacgacgatattgtgaaggagccggacagcgataccaccttcatttacaacatgagcaagcaggattttatcgcggcgggtctgaacgaggacgaagcgaacaaatatgaggcgatcgtgaaggaagcgaaacgttataaaaacgagtacaacctgaagaaaccgctggcggaacacattaacctgaccgactacgataaccaggttgcgcaagataccagcagcctgattaacgacggtgtgaaagttcaacgtaccgagaccattaagagcaacgatatcaaccagctgaccgtggcgaccgatccgcacttcaactttgaaggtgacattaaaatcaacggcaagaaatacgacatcaaggatcagagcgtgcaactggacaccagcaacaaagagtataaggtggaagttaacggtgttgcgaaactgaagaaagacgcggagaaggatttcctgaaggacaaaaccatgcacctgcagctgctgtttggccaggcgaaccgtcaagatgaaccgaacgacaagaaagcgaccagcgtggttgacgttaccctgaaccacaacctggatggtcgtctgagcaaggacgcgctgagccagcaactgagcgcgctgagccgtttcgatgcgcactataaaatgtacaccgacaccaagggccgtgaggataaaccgtttgacaacaagcgtctgattgatatgatggtggaccaagttatcaacgacatggaaagcttcaaagacgataaggtggcggttctgcaccagatcgatagcatggaggaaaacagcgacaaactgattgacgatatcctgaacaacaagaaaaacaccaccaagaacaaagaggatattagcaagctgatcgaccaactggaaaacgttaagaaaaccttcgcggaggaaccgcaggagccgaaaatcgataagggtaaaaacgacgaatttaacaccatgagcagcaacctggacaaagagattagccgtatcagcgaaaagagcacccaactgctgagcgatacccaggagagcaagaccattgcggacagcgtgagcggccagctgaaccaactggataacaacgttaacaagctgcatgcgaccggtcgtgcgctgggtgtgcgtgcgaacgacctgaaccgtcaaatggcgaaaaacgacaaggataacgagctgttcgcgaaagaatttaagaaagttctgcagaacagcaaggacggtgatcgtcagaaccaagcgctgaaagcgttcatgagcaacccggtgcaaaagaaaaacctggaaaacgttctggcgaacaacggcaacaccgatggtagcggtggcggtgcgaaagtggcgaagcagggtcaatacaagaaccaggacccgattgtgctggttcacggcttcaacggttttaccgacgatatcaacccgagcgttctggcgcactattggggcggtaacaagatgaacattcgtcaggacctggaggaaaacggttataaagcgtacgaggcgagcatcagcgcgtttggcagcaactacgatcgtgcggtggaactgtactattacatcaagggcggtcgtgttgactatggtgcggcgcatgcggcgaaatatggccacgagcgttatggcaagacctacgaaggcatctataaagactggaagccgggtcaaaaagtgcacctggttggccacagcatgggcggtcagaccattcgtcaactggaggaactgctgcgtaacggtagccgtgaggaaattgagtatcagaagaaacacggcggtgaaatcagcccgctgttcaaaggcaacaacgataacatgattagcagcatcaccaccctgggtaccccgcacaacggtacccatgcgagcgacctggcgggtaacgaggcgctggtgcgtcagattgttttcgatatcggcaaaatgtttggtaacaagaacagccgtgtggacttcggtctggcgcagtggggcctgaagcaaaaaccgaacgaaagctatatcgattacgtgaaacgtgttaagcagagcaacctgtggaagagcaaagataacggtttttatgacctgacccgtgagggtgcgaccgacctgaaccgtaagaccagcctgaacccgaacattgtgtataaaacctacaccggtgaagcgacccacaaagcgctgaacagcgatcgtcagaaggcggacctgaacatgttctttccgttcgttattaccggcaacctgatcggcaaggcgaccgagaaagaatggcgtgagaacgacggcctggttagcgttatcagcagccagcacccgtttaaccaagcgtacaccaacgcgaccgataaaattcagaagggtatctggcaagtgaccccgaccaagcacgactgggatcacgtggacttcgttggccaagacagcagcgataccgttcgtacccgtgaggaactgcaggatttttggcaccacctggcggacgatctggtgaaaaccgaaaaggttaccgacaccaaacaa

SEQ ID NO: 74 - DNA, 2415 bp, Artificial sequence
  Fusion protein encoding DNA
atggatataggtaagaaacacgtaatacccaagtcacaatacaggagaaaacgtcgtgagttctttcacaacgaagaccgtgaggaaaacctgaaccagcaccaagataagcagaacatcgacaacaccaccagcaagaaagcggataagcaaatccacaaagatagcattgacaagcacgagcgtttcaaaaacagcctgagcagccacctggagcagcgtaaccgtgacgtgaacgaaaacaaggcggaggaaagcaaaagcaaccaagacagcaaaagcgcgtacaaccgtgatcactatctgaccgacgatgtgagcaagaaacagaacagcctggatagcgttgatcaagacaccgaaaagagcaaatactatgagcagaacagcgaagcgaccctgagcaccaagagcaccgacaaagttgagagcaccgaaatgcgtaagctgagcagcgataagaacaaagtgggtcacgaggaacagcacgttctgagcaagccgagcgagcacgataaagaaacccgtatcgacagcgagagcagccgtaccgatagcgacagcagcatgcaaaccgaaaagattaagaaagatagcagcgacggcaacaagagcagcaacctgaaaagcgaggtgatcagcgacaagagcaacaccgttccgaaactgagcgagagcgacgatgaagttaacaaccagaaaccgctgaccctgccggaggaacaaaagctgaaacgtcagcaaagccagaacgagcaaaccaagacctatacctacggtgatagcgagcagaacgacaaaagcaaccacgaaaacgacctgagccaccacatcccgagcattagcgacgataaggataacgtgatgcgtgaaaaccacatcgttgacgataacccggataacgacattaacaccccgagcctgagcaaaaccgacgatgaccgtaagctggatgagaaaattcacgtggaagacaagcacaaacagaacgcggatagcagcgagaccgttggctaccagagccaaagcaccgcgagccaccgtagcaccgaaaagcgtaacatcagcattaacgatcacgacaaactgaacggtcagaagaccaacaccaaaaccagcgcgaacaacaaccaaaagaaagcgaccagcaagctgaacaaaggccgtgcgaccaacaacaactacagcgacatcctgaagaaattctggatgatgtattggccgaagggtagcggtggcggtgcgaaggtggcgaaacagggccaatacaaaaaccaggatccgatcgtgctggttcacggcttcaacggttttaccgatgacattaacccgagcgttctggcgcactattggggcggtaacaagatgaacatccgtcaagacctggaggaaaacggttataaagcgtacgaggcgagcattagcgcgtttggcagcaactacgaccgtgcggtggaactgtactattacatcaagggcggtcgtgttgattatggtgcggcgcatgcggcgaagtatggtcatgagcgttatggtaaaacctacgaaggcatttataaggactggaaaccgggtcagaaagtgcacctggttggccacagcatgggcggtcagaccatccgtcaactggaggaactgctgcgtaacggtagccgtgaggaaatcgagtatcaaaagaaacacggcggtgaaattagcccgctgtttaaaggcaacaacgacaacatgatcagcagcattaccaccctgggtaccccgcacaacggtacccatgcgagcgatctggcgggtaacgaggcgctggtgcgtcagatcgttttcgacattggcaagatgtttggtaacaaaaacagccgtgtggatttcggtctggcgcagtggggcctgaagcaaaaaccgaacgaaagctatattgactacgtgaagcgtgttaaacaaagcaacctgtggaagagcaaagataacggtttttacgacctgacccgtgagggcgcgaccgatctgaaccgtaagaccagcctgaacccgaacatcgtgtataaaacctacaccggtgaagcgacccacaaggcgctgaacagcgatcgtcagaaagcggacctgaacatgttctttccgttcgttatcaccggcaacctgattggcaaggcgaccgagaaagaatggcgtgagaacgacggcctggttagcgttatcagcagccagcacccgtttaaccaagcgtacaccaacgcgaccgacaagatccagaaaggtatttggcaagtgaccccgaccaagcacgattgggaccacgtggatttcgttggccaggatagcagcgacaccgttcgtacccgtgaggaactgcaagacttttggcaccacctggcggatgacctggtgaagaccgaaaaagttaccgataccaaacaa

SEQ ID NO: 75 - DNA, 2637 bp, Artificial sequence
  Fusoin protein encoding DNA
gatacaccccaaaaagatacaactgctaaaacaactagtcacgatagcaagaaaagcaacgacgatgagaccagcaaggacaccaccagcaaagacatcgataaggcggataacaacaacaccagcaaccaggacaacaacgataagaaattcaagaccattgacgatagcaccagcgacagcaacaacatcatcgacttcatctacaagaacctgccgcagaccaacattaaccaactgctgaccaagaacaaatacgacgataactatagcctgaccaccctgatccagaacctgttcaacctgaacagcgacattagcgattatgagcaaccgcgtaacggcgagaagagcaccaacgatagcaacaagaacagcgacaacagcatcaaaaacgacaccgatacccagagcagcaaacaagacaaggcggataaccagaaagcgccgaagagcaataacaccaagccgagcaccagcaacaaacaaccgaacagcccgaagccgacccagccgaaccaaagcaacagccaaccggcgagcgacgataaagcgaaccagaagagcagcagcaaagacaaccaaagcatgagcgacagcgcgctggatagcatcctggaccagtacagcgaagatgcgaagaaaacccagaaggactatgcgagccaaagcaagaaagacaaaaacgagaagagcaacaccaaaaacccgcagctgccgacccaagatgaactgaagcacaaaagcaagccggcgcagagcttcaacaacgatgttaaccaaaaggacacccgtgcgaccagcctgtttgagaccgacccgagcatcagcaacaacgacgatagcggccagttcaacgtggttgacagcaaagatacccgtcaatttgttaagagcattgcgaaagacgcgcaccgtatcggccaggacaacgatatttacgcgagcgtgatgatcgcgcaagcgattctggaaagcgatagcggtcgtagcgcgctggcgaagagcccgaaccacaacctgttcggcatcaagggtgcgtttgagggcaacagcgtgccgttcaacaccctgcaggcggacggcaacaagctgtacagcattaacgcgggttttcgtaaatatccgagcaccaaggagagcctgaaaaactacagcgatctgatcaagaacggcattgacggtaaccgtaccatctacaagccgacctggaaaagcgaagcggatagctataaggacgcgaccagccacctgagcaaaacctacgcgaccgatccgaactatgcgaagaaactgaacagcatcattaaacactaccagctgacccaattcgacgatgagcgtatgccggacctggataaatacgaacgtagcatcaaggattatgacgatagcagcgacgagttcaagccgtttcgtgaagttagcgacagcatgccgtatccgcacggccagagcacctggtacgtttataaccgtatgaaacaatttggtaccagcattagcggcgacctgggtgatgcgcacaactggaacaaccgtgcgcagtaccgtgactatcaagttagccataccccgaaacgtcatgcggcggtggtttttgaggcgggccagtttggtgcggaccaacactacggccacgtggcgttcgttgagaaggtgaacagcgatggtagcatcgttattagcgaaagcaacgtgaaaggcctgggtatcattagccatcgtaccatcaacgcggcggcggcggaggaactgagctatattaccggcaaaggtagcggtggcggtgcggacattggtagcaacaccaccgttaagaccggtgatctggtgacctacgacaaagaaaacggtatgctgaagaaagttttctatagcttcatcgacgataaaaaccacaacaagaaactgctggttatccgtaccaaaggcaccattgcgggccagtaccgtgtgtatagcgaggaaggcgcgaacaagagcggtctggcgtggccgagcgcgttcaaagttcagctgcaactgccggataacgaggtggcgcaaatcagcgactactatccgcgtaacagcattgataccaaggaatacatgagcaccctgacctatggctttaacggtaacgtgaccggcgacgataccggtaaaatcggcggtctgattggcgcgaacgttagcattggtcacaccctgaagtacgtgcagccggacttcaaaaccatcctggagagcccgaccgataagaaagttggctggaaagtgatttttaacaacatggttaaccagaactggggtccgtacgaccgtgatagctggaacccggtgtatggcaaccaactgttcatgaagacccgtaacggtagcatgaaagcggcggataactttctggacccgaacaaagcgagcagcctgctgagcagcggtttcagcccggactttgcgaccgtgatcaccatggatcgtaaagcgagcaagcagcaaaccaacatcgacgttatttacgaacgtgtgcgtgacgattatcagctgcactggaccagcaccaactggaaaggcaccaacaccaaggataagtggatcgaccgcagcagcgagcgttacaagattgattgggaaaaagaggaaatgaccaac

SEQ ID NO: 76 - DNA, 2313 bp, Artificial sequence
  Fusion protein encoding dna
atcgacagcaagaacaaaccggcgaacagcgatattaagtttgaagtgacccagaagagcgatgcggttaaagcgctgaaggaactgccgaagagcgagaacgtgaaaaacatctaccaggactatgcggtgaccgacgttaagaccgataagaaaggtttcacccactacaccctgcagccgagcgtggacggtgttcatgcgccggataaagaggtgaaggttcacgcggacaaaagcggcaaggtggttctgatcaacggtgacaccgatgcgaagaaagtgaagccgaccaacaaagttaccctgagcaaagacgatgcggcggacaaagcgttcaaggcggttaagatcgataagaacaaggcgaagaacctgaaagacaaggtgatcaaggagaacaaagttgaaattgacggtgatagcaacaagtacgtgtataacgttgaactgatcaccgtgaccccggagattagccactggaaagttaagatcgacgcgcagaccggcgagattctggaaaagatgaacctggtgaaagaggcggcggaaaccggcaaaggcaagggcgttctgggcgacaccaaagatatcaacattaacagcatcgacggtggcttcagcctggaagatctgacccaccagggcaagctgagcgcgttcagctttaacgaccagaccggccaagcgaccctgattaccaacgaggacgaaaactttgtgaaagatgaacagcgtgcgggtgttgatgcgaactactatgcgaaacaaacctacgactactataaggataccttcggccgtgagagctatgacaaccagggtagcccgatcgtgagcctgacccacgttaacaactacggtggccaagataaccgtaacaacgcggcgtggatcggtgacaagatgatttatggtgacggcgatggtcgtaccttcaccagcctgagcggtgcgaacgatgttgttgcgcatgcgctgacccatggtgtgacccaggaaaccgcgaacctggagtacaaagatcaaagcggcgcgctgaacgaaagcttcagcgacgtgttcggttactttgttgacgatgaagactttctgatgggcgaggatgtttataccccgggcaaggaaggtgacgcgctgcgtagcatgagcaacccggagcagttcggtcaaccggcgcacatgaaagactacgtgtttaccgagaaggataacggtggcgttcacaccaacagcggtatcccgaacaaagcggcgtataacgtgatccaggcgattggcaaaagcaagagcgaacaaatttactatcgtgcgctgaccgagtacctgaccagcaacagcaactttaaggacagcaaagatgcgctgtaccaggcggcgaaagacctgtatgatgagcagaccgcggaacaagtttatgaggcgtggaacgaagtgggcgttgagggtagcggtggcggtgcgaacaccaacatcgaaaacattggcgatggtgcggaagtgatcaagcgtaccgaggacgttagcagcaagaaatggggtgtgacccagaacgttcaattcgacttcgtgaaggataagaagtacaacaaggatgcgctgatcgttaagatgcagggcttcattaacagccgtaccagctttagcgacgtgaaaggcagcggttacgaactgaccaagcgtatgatctggccgttccaatataacattggtctgaccaccaaagatccgaacgtgagcctgatcaactacctgccgaaaaacaagattgaaaccaccgacgttggccagaccctgggttataacatcggcggtaacttccaaagcgcgccgagcattggcggtaacggcagctttaactacagcaaaaccatcagctacacccagaagagctatgtgagcgaagttgataaacaaaacagcaagagcgtgaaatggggtgttaaggcgaacgagttcgtgaccccggacggcaagaaaagcgcgcacgatcgttacctgtttgttcaaagcccgaacggtccgaccggtagcgcgcgtgaatattttgcgccggacaaccagctgccgccgctggtgcaaagcggtttcaacccgagctttattaccaccctgagccacgagaagggtagcagcgataccagcgagtttgaaatcagctacggccgtaacctggacattacctatgcgaccctgttcccgcgtaccggtatctacgcggagcgtaaacacaacgcgttcgttaaccgtaactttgtggttcgttatgaagtgaactggaagacccacgagattaaagttaagggccacaac

SEQ ID NO: 77 - DNA, 1710 bp, Artificial sequence
  Fusion protein encoding DNA
aacaccaacatcgagaacattggcgatggtgcggaagtgattaagcgtaccgaagacgttagcagcaagaaatggggcgtgacccagaacgttcaattcgacttcgtgaaggataagaagtacaacaaggatgcgctgatcgttaagatgcagggtttcattaacagccgtaccagctttagcgacgttaaaggcagcggttacgagctgaccaagcgtatgatctggccgttccaatataacattggcctgaccaccaaagatccgaacgtgagcctgatcaactacctgccgaagaacaaaattgaaaccaccgacgttggccagaccctgggttataacatcggtggcaacttccaaagcgcgccgagcattggtggcaacggtagctttaactacagcaaaaccatcagctacacccagaagagctatgtgagcgaggttgataaacaaaacagcaagagcgtgaaatggggcgttaaggcgaacgaattcgtgaccccggacggtaagaaaagcgcgcacgatcgttacctgtttgttcaaagcccgaacggtccgaccggtagcgcgcgtgagtattttgcgccggacaaccagctgccgccgctggtgcaaagcggcttcaacccgagctttattaccaccctgagccacgaaaagggcagcagcgataccagcgagtttgaaatcagctacggtcgtaacctggacattacctatgcgaccctgttcccgcgtaccggtatctacgcggagcgtaaacacaacgcgttcgttaaccgtaactttgtggttcgttatgaggtgaactggaagacccacgaaatcaaggttaaaggtcataacggtagcggtggcggtgcggacattggcagcaacaccaccgtgaaaaccggtgacctggttacctacgataaggaaaacggtatgctgaagaaagtgttctatagctttatcgacgataaaaaccacaacaagaaactgctggtgatccgtaccaagggcaccattgcgggccagtaccgtgtttatagcgaggaaggcgcgaacaaaagcggtctggcgtggccgagcgcgtttaaggtgcagctgcaactgccggacaacgaggttgcgcaaatcagcgattactatccgcgtaacagcattgacaccaaagaatacatgagcaccctgacctatggcttcaacggtaacgttaccggcgacgataccggcaagatcggcggtctgattggcgcgaacgtgagcatcggtcacaccctgaaatacgttcagccggattttaagaccattctggagagcccgaccgacaagaaagtgggctggaaagttatcttcaacaacatggtgaaccagaactggggtccgtacgaccgtgatagctggaacccggtttatggcaaccaactgttcatgaaaacccgtaacggtagcatgaaggcggcggacaactttctggatccgaacaaggcgagcagcctgctgagcagcggtttcagcccggattttgcgaccgtgatcaccatggaccgtaaggcgagcaaacagcaaaccaacatcgatgtgatttacgaacgtgttcgtgacgattatcaactgcactggaccagcaccaactggaaaggtaccaacaccaaggacaaatggatcgatcgtagcagcgagcgttacaagattgactgggaaaaagaggaaatgaccaac

SEQ ID NO: 78 - DNA, 2337 bp, Artificial sequence
  Fusion protein encoding DNA
atagattcaaagaacaagcccgctaacagtgatataaagttcgaagtgacccagaaaagcgatgcggttaaagcgctgaaggaactgccgaagagcgagaacgtgaaaaacatctaccaggactatgcggtgaccgacgttaaaaccgataagaaaggttttacccactacaccctgcagccgagcgtggacggtgttcatgcgccggataaagaggtgaaggttcacgcggacaaaagcggcaaggtggttctgatcaacggtgacaccgatgcgaagaaagtgaagccgaccaacaaagttaccctgagcaaggacgatgcggcggacaaagcgttcaaggcggttaaaattgataagaacaaagcgaagaacctgaaagacaaggtgatcaaggagaacaaagttgaaattgacggtgatagcaacaaatacgtgtataacgttgaactgatcaccgtgaccccggagattagccactggaaagttaagatcgatgcgcagaccggcgagattctggaaaagatgaacctggtgaaagaggcggcggaaaccggcaaaggcaagggcgttctgggcgacaccaaggatatcaacattaacagcatcgacggtggctttagcctggaggatctgacccaccaaggtaaactgagcgcgttcagctttaacgaccagaccggccaagcgaccctgattaccaacgaggacgaaaacttcgtgaaagatgaacagcgtgcgggtgttgatgcgaactactatgcgaagcaaacctacgactactataaagatacctttggccgtgagagctatgacaaccagggtagcccgatcgtgagcctgacccacgttaacaactacggtggccaagataaccgtaacaacgcggcgtggatcggcgacaagatgatttatggtgacggcgatggtcgtaccttcaccagcctgagcggtgcgaacgatgttgttgcgcatgcgctgacccatggtgtgacccaggaaaccgcgaacctggagtacaaggatcaaagcggcgcgctgaacgaaagcttcagcgacgtgttcggttactttgttgacgatgaagactttctgatgggcgaggatgtttataccccgggcaaagaaggtgacgcgctgcgtagcatgagcaacccggagcagttcggtcaaccggcgcacatgaaggactacgtgtttaccgagaaagataacggtggcgttcacaccaacagcggtatcccgaacaaggcggcgtataacgtgatccaggcgattggcaaaagcaagagcgaacaaatttactatcgtgcgctgaccgagtacctgaccagcaacagcaacttcaaggacagcaaagatgcgctgtaccaggcggcgaaggacctgtatgatgagcagaccgcggaacaagtttatgaggcgtggaacgaagtgggcgttgagggtagcggtggcggtgcggcgcaggtggacgaagcgcagaagaacgcgttctaccaggttctgaacatgccgaacctgaacgcggatcagcgtaacggttttatccaaagcctgaaggatgcgccgagccagagcgcgaaggtgctgggtgaagcgcagaaactgaacgacagccaagcgccgaaagcggatgcgcagcaaaacaacgttaacaaggaccagaaaagcgcgttctatgaaatcctgaatatgccgaatctgaacgaggcgcagcgtaacggttttattcagagcctgaaagatgcgccgagccaaagcaccaaagtgctgggcgaagcgaagaaactgaacgagagccaggcgccgaaggcggacaacaacgttaacaaggaacagaagaacgcgttttatgagatcctgaatatgccgaacctgaacgaggaacaacgcaacggttttattcaaagcctgaaagacgcgccgagccaaagcgcgaaactgctgagcgaggcgaagaaactgaatgagagccaggcgccgaaagcggacaacaaggtgaacaaagaacaaaagaacgcgttctatgagatcctgcacctgccgaatctgaatgaagaacagcgtaacggctttattcagtctttaaaagatgctcctagccagagcgcgaaactgctggcggaggcgaagaaactgaacgatgcgcaggcgccgaaggcggataacaaagtgaataaagaacagaagaacgcgttctatgaaattctgcacctgccgaacctgaccgaggaacaacgtaatggcttcattcagagcctgaaggacgcgccgagcgttagcaagaaaattctggcggaagcgaagaaactgaatgacgcgcaggcgccgaaa

SEQ ID NO: 79 - DNA, 3246 bp, Artificial sequence
  Fusion protein encoding DNA
aacaccaacattgagaacatcggcgatggtgcggaagtgatcaagcgtaccgaagacgttagcagcaagaaatggggcgtgacccagaacgttcaattcgacttcgtgaaggataagaagtacaacaaggatgcgctgattgttaaaatgcaaggtttcatcaacagccgtaccagctttagcgacgtgaagggcagcggttacgagctgaccaaacgtatgatttggccgttccagtataacatcggcctgaccaccaaagatccgaacgtgagcctgattaactacctgccgaagaacaaaatcgaaaccaccgacgttggccaaaccctgggttataacattggtggcaactttcaaagcgcgccgagcatcggtggcaacggtagctttaactacagcaagaccatcagctacacccagaagagctacgtgagcgaggttgataaacagaacagcaagagcgtgaaatggggcgttaaggcgaacgaattcgtgaccccggatggtaagaaaagcgcgcacgaccgttacctgtttgttcaaagcccgaacggtccgaccggtagcgcgcgtgagtattttgcgccggacaaccagctgccgccgctggtgcaaagcggcttcaacccgagctttattaccaccctgagccacgaaaaaggcagcagcgataccagcgagtttgaaattagctacggtcgtaacctggacatcacctatgcgaccctgttcccgcgtaccggtatctacgcggagcgtaaacacaacgcgttcgttaaccgtaactttgtggttcgttatgaggtgaactggaagacccacgaaattaaggttaaaggtcataacggtagcggtggcggtgcgcagaccaagtatggcgatcaaagcgaaaaaggtagccagagcgtgagcaacaagaacaacaaaatccacattgcgatcgttaacgaggaccaaccgaccacctataacggcaagaaagtggaactgggtcaggcgttcatcaagcgtctggcgaacgagaagaactacaaatttgaaaccgtgacccgtaacgttgcggagagcggcctgaaaaacggcggttatcaagtgatgattgttatcccggaaaactttagcaagctggcgatgcagctggacgcgaaaaccccgagcaagattagcctgcaatacaagaccgcggtgggtcagaaagaggaagttgcgaaaaacaccgagaaggtggttagcaacgtgctgaatgatttcaacaagaacctggttgaaatctacctgaccagcatcattgacaacctgcacaacgcgcagaaaaacgtgggcgcgatcatgacccgtgagcacggtgttaacagcaagttcagcaactatctgctgaacccgattaacgatttcccggaactgtttaccgacaccctggttaacagcattagcgcgaacaaagatatcaccaagtggtttcaaacctacaacaagagcctgctgagcgcgaacagcgataccttccgtgtgaacaccgactataacgttagcaccctgatcgagaaacagaacagcctgttcgacgaacacaacaccgcgatggataagatgctgcaggactacaagagccaaaaagatagcgtggagctggacaactatattaacgcgctgaaacagatggacagccaaatcgatcagcaaagcagcatgcaagataccggcaaagaggaatacaagcagaccgttaaagagaacctggacaagctgcgtgagatcattcagagccaagaaagcccgtttagcaagggtatgatcgaagattatcgtaaacaactgaccgagagcctgcaggacgaactggcgaacaacaaggacctgcaggatgcgctgaacagcatcaaaatgaacaacgcgcaattcgcggagaacctggaaaagcagctgcacgacgatattgtgaaagaaccggacagcgataccaccttcatttacaacatgagcaaacaggattttatcgcggcgggtctgaacgaggacgaagcgaacaagtatgaggcgatcgtgaaggaagcgaaacgttacaagaacgagtataacctgaagaaaccgctggcggaacacattaacctgaccgactacgataaccaggttgcgcaagataccagcagcctgatcaacgacggtgtgaaggttcaacgtaccgaaaccattaaaagcaacgatatcaaccagctgaccgtggcgaccgatccgcacttcaactttgaaggcgacattaagatcaacggtaagaaatacgacatcaaagatcagagcgtgcaactggacaccagcaacaaagagtataaggtggaagttaacggcgttgcgaagctgaagaaagacgcggagaaagatttcctgaaggacaaaaccatgcacctgcagctgctgtttggtcaggcgaaccgtcaagatgaaccgaacgacaagaaagcgaccagcgtggttgacgttaccctgaaccacaacctggatggccgtctgagcaaagacgcgctgagccagcaactgagcgcgctgagccgttttgatgcgcactacaagatgtataccgacaccaagggtcgtgaggataaaccgttcgacaacaagcgtctgattgatatgatggtggaccaagttatcaacgacatggaaagcttcaaagacgataaggtggcggttctgcaccagatcgatagcatggaggaaaacagcgacaagctgattgacgatatcctgaacaacaagaaaaacaccaccaagaacaaagaggatattagcaaactgatcgaccaactggaaaacgttaagaaaacctttgcggaggaaccgcaggagccgaagatcgataagggcaaaaacgacgaattcaacaccatgagcagcaacctggacaaggagattagccgtatcagcgagaagagcacccaactgctgagcgatacccaggagagcaaaaccattgcggacagcgtgagcggtcagctgaaccaactggataacaacgttaacaagctgcatgcgaccggtcgtgcgctgggtgtgcgtgcgaacgacctgaaccgtcaaatggcgaaaaacgacaaggataacgagctgttcgcgaaggaatttaagaaagttctgcagaacagcaaagacggcgatcgtcagaaccaagcgctgaaggcgttcatgagcaacccggtgcagaagaaaaacctggaaaacgttctggcgaacaacggtaacaccgac

SEQ ID NO: 80 - DNA, 2088 bp, Artificial sequence
  Fusion protein encoding DNA
atggacatcggtaagaaacacgtgattccgaagagccagtaccgtcgtaaacgtcgtgagttctttcacaacgaagatcgtgaggaaaacctgaaccagcaccaagataagcagaacatcgacaacaccaccagcaagaaagcggacaagcaaatccacaaagatagcattgacaagcacgagcgtttcaaaaacagcctgagcagccacctggagcagcgtaaccgtgatgtgaacgaaaacaaggcggaggaaagcaaaagcaaccaagacagcaaaagcgcgtacaaccgtgatcactatctgaccgacgatgtgagcaagaaacagaacagcctggacagcgttgatcaagacaccgaaaagagcaaatactatgagcagaacagcgaagcgaccctgagcaccaagagcaccgataaagttgagagcaccgaaatgcgtaagctgagcagcgacaagaacaaagtgggtcacgaggaacagcacgttctgagcaagccgagcgagcacgataaagaaacccgtattgacagcgagagcagccgtaccgatagcgacagcagcatgcaaaccgaaaagatcaagaaagatagcagcgacggcaacaagagcagcaacctgaaaagcgaggtgatcagcgacaagagcaacaccgttccgaaactgagcgagagcgacgatgaagttaacaaccagaaaccgctgaccctgccggaggaacaaaagctgaaacgtcagcaaagccagaacgagcaaaccaagacctacacctatggtgatagcgagcagaacgacaaaagcaaccacgaaaacgatctgagccaccacatcccgagcattagcgacgataaggacaacgtgatgcgtgaaaaccacatcgttgacgataacccggataacgacattaacaccccgagcctgagcaaaaccgacgatgaccgtaagctggatgagaaaatccacgtggaagacaagcacaaacaaaacgcggatagcagcgaaaccgttggctaccagagccaaagcaccgcgagccaccgtagcaccgaaaagcgtaacatcagcattaacgatcacgacaaactgaacggtcagaagaccaacaccaaaaccagcgcgaacaacaaccaaaagaaagcgaccagcaagctgaacaaaggccgtgcgaccaacaacaactacagcgatatcctgaagaaattctggatgatgtactggccgaaaggtagcggtggcggtgcggacattggtagcaacaccaccgtgaagaccggcgatctggttacctacgacaaagaaaacggtatgctgaagaaagtgttctatagctttattgatgacaagaaccacaacaagaaactgctggtgatccgtaccaaaggcaccattgcgggccagtaccgtgtttatagcgaggaaggcgcgaacaagagcggtctggcgtggccgagcgcgttcaaagtgcagctgcaactgccggacaacgaggttgcgcaaatcagcgattactatccgcgtaacagcattgacaccaaggaatacatgagcaccctgacctatggctttaacggtaacgttaccggcgatgacaccggtaaaatcggcggtctgattggcgcgaacgtgagcatcggtcacaccctgaagtacgttcagccggatttcaaaaccattctggagagcccgaccgacaagaaagtgggttggaaggttatctttaacaacatggtgaaccagaactggggcccgtacgatcgtgacagctggaacccggtttatggtaaccaactgttcatgaagacccgtaacggcagcatgaaagcggcggataactttctggacccgaacaaagcgagcagcctgctgagcagcggtttcagcccggattttgcgaccgtgatcaccatggaccgtaaggcgagcaaacagcaaaccaacatcgacgtgatttacgaacgtgttcgtgatgactatcagctgcactggaccagcaccaactggaagggcaccaacaccaaggataaatggatcgaccgtagcagcgagcgttataagattgactgggaaaaagaggaaatgaccaac

SEQ ID NO: 81 - DNA, 2289 bp, Artificial sequence
  Fusion protein encoding DNA
aataacgtaaacaaaaacgacagaatgaacgacaataatgacgcagacgctcaaaaatacaccactacaatgaagaacgctaacaacacagttaaatcagttgttacagttgaaaacgaaacttcaaaggattcttcattgccaaaggataaagcatctcaagatgaagttggttcaggtgttgtttacaaaaaatctggtgatacattgtacatcgttactaacgcacatgttgttggtgataaagaaaaccaaaagatcacattctctaacaacaaatcagttgttggcaaggttttgggcaaggataagtggtctgatttagctgttgttaaagcaacttcttcagattcttcagttaaggaaatcgctatcggtgattcaaacaacttggttttgggtgaaccaatcttggttgttggtaatccattaggtgttgatttcaagggtactgttacagaaggtatcatctctggtttgaacagaaatgttccaatcgatttcgataaggataacaagtacgatatgttgatgaaagcatttcaaatcgatgcatcagttaaccctggtaatgcaggtggtgctgttgttaatagagagggtaaattgattggtgttgttgctgcaaagatctctatgccaaacgttgaaaacatgtcattcgctatcccagttaacgaagttcaaaagatcgttaaggatttagaaactaaaggtaaaattgattacccagatgttggtgttaagatgaagaacatcgcttctttaaattctttcgaaagacaagcagttaaattgccaggcaaggttaagaacggtgttgttgttgatcaagttgataacaacggtttggctgatcaatctggtttgaagaaaggtgatgttatcacagaattggatggtaaattgttagaagatgatttaagatttagacaaattattttctctcataaagatgatttgaaatcaattactgcaaaaatttatagagatggcaaggaaaaggaaatcaacatcaaattgaaaggttctggtggtggtgctggttctggtggtggtgctatggatatcggtaaaaaacatgttatcccaaagtctcaatacagaagaaagagaagagaatttttccataacgaagatagagaagaaaatttgaatcaacatcaagataagcaaaacatcgataacactacttctaagaaagctgataagcaaatccataaggattctatcgataaacatgaaagatttaaaaattcattgtcttcacatttagaacaaagaaacagagatgttaacgaaaacaaagctgaagaatctaagtcaaaccaagattctaagtcagcatacaacagagatcattacttgacagatgatgtttctaaaaaacaaaattctttggattcagttgatcaagatactgaaaaatctaagtactacgaacaaaattcagaagcaactttatctacaaaatcaactgataaggttgaatctacagaaatgagaaaattgtcttcagataagaacaaggttggtcatgaagaacaacatgttttgtctaagccatcagaacatgataaggaaactagaatcgattctgaatcttcaagaacagattcagattcttcaatgcaaactgaaaagattaagaaagattcttctgatggtaacaagtcttcaaacttgaagtctgaagttatttctgataaatcaaatacagttccaaaattgtctgaatcagatgatgaagttaacaaccaaaaaccattgactttgccagaagaacaaaaattgaagagacaacaatctcaaaacgaacaaactaagacatacacttacggtgattctgaacaaaacgataagtcaaaccatgaaaacgatttatcacatcatattccatctatctcagatgataaggataacgttatgagagaaaaccatatcgttgatgataacccagataacgatatcaacacaccatctttgtcaaagactgatgatgatagaaaattggatgaaaagatccatgttgaagataagcataaacaaaatgctgattcttcagaaacagttggttatcaatctcaatcaacagcatctcatagatcaactgaaaagagaaacatctctatcaacgatcatgataaattgaatggtcaaaagacaaacactaagacatctgctaacaataatcaaaagaaagctacttcaaaattgaataaaggtagagcaactaacaacaactactccgacatattgaaaaaattctggatgatgtattggcctaaa

SEQ ID NO: 82 - protein, 580 aa, Artificial Sequence
  Fusion protein
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHNGSGGGAAQVDEAQKNAFYQVLNMPNLNADQRNGFIQSLKDAPSQSAKVLGEAQKLNDSQAPKADAQQNNVNKDQKSAFYEILNMPNLNEAQRNGFIQSLKDAPSQSTKVLGEAKKLNESQAPKADNNVNKEQKNAFYEILNMPNLNEEQRNGFIQSLKDAPSQSAKLLSEAKKLNESQAPKADNKVNKEQKNAFYEILHLPNLNEEQRNGFIQSLKDAPSQSAKLLAEAKKLNDAQAPKADNKVNKEQKNAFYEILHLPNLTEEQRNGFIQSLKDAPSVSKKILAEAKKLNDAQAPK

SEQ ID NO: 83 - protein, 889 aa, Artificial Sequence
  Fusion protein
DTPQKDTTAKTTSHDSKKSNDDETSKDTTSKDIDKADNNNTSNQDNNDKKFKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKANQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLQADGNKLYSINAGFRKYPSTKESLKNYSDLIKNGIDGNRTIYKPTWKSEADSYKDATSHLSKTYATDPNYAKKLNSIIKHYQLTQFDDERMPDLDKYERSIKDYDDSSDEFKPFREVSDSMPYPHGQSTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGKGSGGGAAQVDEAQKNAFYQVLNMPNLNADQRNGFIQSLKDAPSQSAKVLGEAQKLNDSQAPKADAQQNNVNKDQKSAFYEILNMPNLNEAQRNGFIQSLKDAPSQSTKVLGEAKKLNESQAPKADNNVNKEQKNAFYEILNMPNLNEEQRNGFIQSLKDAPSQSAKLLSEAKKLNESQAPKADNKVNKEQKNAFYEILHLPNLNEEQRNGFIQSLKDAPSQSAKLLAEAKKLNDAQAPKADNKVNKEQKNAFYEILHLPNLTEEQRNGFIQSLKDAPSVSKKILAEAKKLNDAQAPK

SEQ ID NO: 84 - protein, 706 aa, Artificial Sequence
  Fusion protein
MDIGKKHVIPKSQYRRKRREFFHNEDREENLNQHQDKQNIDNTTSKKADKQIHKDSIDKHERFKNSLSSHLEQRNRDVNENKAEESKSNQDSKSAYNRDHYLTDDVSKKQNSLDSVDQDTEKSKYYEQNSEATLSTKSTDKVESTEMRKLSSDKNKVGHEEQHVLSKPSEHDKETRIDSESSRTDSDSSMQTEKIKKDSSDGNKSSNLKSEVISDKSNTVPKLSESDDEVNNQKPLTLPEEQKLKRQQSQNEQTKTYTYGDSEQNDKSNHENDLSHHIPSISDDKDNVMRENHIVDDNPDNDINTPSLSKTDDDRKLDEKIHVEDKHKQNADSSETVGYQSQSTASHRSTEKRNISINDHDKLNGQKTNTKTSANNNQKKATSKLNKGRATNNNYSDILKKFWMMYWPKGSGGGAAQVDEAQKNAFYQVLNMPNLNADQRNGFIQSLKDAPSQSAKVLGEAQKLNDSQAPKADAQQNNVNKDQKSAFYEILNMPNLNEAQRNGFIQSLKDAPSQSTKVLGEAKKLNESQAPKADNNVNKEQKNAFYEILNMPNLNEEQRNGFIQSLKDAPSQSAKLLSEAKKLNESQAPKADNKVNKEQKNAFYEILHLPNLNEEQRNGFIQSLKDAPSQSAKLLAEAKKLNDAQAPKADNKVNKEQKNAFYEILHLPNLTEEQRNGFIQSLKDAPSVSKKILAEAKKLNDAQAPK

SEQ ID NO: 85 - protein, 580 aa, Artificial Sequence
  Fusion protein
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHNGSGGGAAQVDEAQKNAFYQVLNMPNLNADQRNGFIQSLKDAPAKSANVLGEAQKLNDSQAPKADAQQNNVNKDQKSAFYEILNMPNLNEAQRNGFIQSLKDAPAKSTNVLGEAKKLNESQAPKADNNVNKEQKNAFYEILNMPNLNEEQRNGFIQSLKDAPAKSANLLSEAKKLNESQAPKADNKVNKEQKNAFYEILHLPNLNEEQRNGFIQSLKDAPAKSANLLAEAKKLNDAQAPKADNKVNKEQKNAFYEILHLPNLTEEQRNGFIQSLKDAPAKSKEILAEAKKLNDAQAPK

SEQ ID NO: 86 - DNA, 1740 bp, Artificial Sequence
  DNA encoding fusion protein
aatactaatattgaaaatattggtgatggtgctgaagtaatcaaacgtacggaggatgtaagtagtaagaaatggggcgttactcaaaatgtccaattcgactttgtaaaagataaaaaatataacaaagacgctttaattgttaaaatgcaaggttttattaattccagaacttcattttcagatgtgaagggtagtggatatgaattaactaaacgaatgatttggccattccaatataatataggactgacgactaaagatccaaatgttagcttaatcaattaccttcctaaaaacaaaatagaaactactgatgttggtcaaacattaggatataacattggaggtaatttccagtcagcaccatctataggtggcaatggctcatttaattattctaaaacaattagttatacccaaaagagttatgtcagtgaagtagacaagcaaaactcaaaatctgttaaatggggtgttaaagcaaacgaatttgttacgcctgatggaaaaaaatctgcgcatgatagatatttattcgtacaaagtccaaatggtccaacaggttcagcaagagaatattttgctcctgataatcaattgccacctttagttcaaagtggctttaatccatcgtttatcactacactatcacatgaaaaaggttcaagtgatacgagtgaatttgaaatttcatatggtagaaacttagatattacatatgcgactttattccctagaactggtatttacgcagaaagaaagcataatgcatttgtaaatagaaactttgtagttagatatgaagttaattggaaaacacacgaaattaaagtgaaaggacataatggttctggcggaggggctgcgcaagtggatgaagctcaaaaaaatgctttttatcaagtcttaaatatgcctaacttaaatgctgatcaacgcaatggttttatccaaagccttaaagatgctccaagccaaagtgctaaagttttaggtgaagctcaaaaacttaatgactctcaagctccaaaagctgatgcgcaacaaaataacgtgaacaaagatcaaaaaagcgccttctatgaaatcttgaacatgcctaacttaaacgaagcgcaacgtaacggcttcattcaaagtcttaaagacgctccaagccaaagcactaaagttttaggtgaagctaaaaaattaaacgaatctcaagcaccgaaagctgataacaatgtgaacaaagaacaaaaaaatgctttctatgaaatcttgaatatgcctaacttaaacgaagaacaacgcaatggtttcatccaaagcttaaaagatgctccaagccaaagtgctaaactattgtcagaagctaaaaagttaaatgaatctcaagcaccgaaagcggataacaaagtgaacaaagaacaaaaaaatgctttctatgaaatcttacatttacctaacttaaacgaagaacaacgcaatggtttcatccaaagcctaaaagatgctccaagccaaagcgctaaacttttagcagaagctaaaaagctaaatgatgctcaagcaccaaaagctgacaacaaagtgaacaaagaacaaaaaaatgctttctatgaaattttacatttacctaacttaactgaagaacaacgtaacggcttcatccaaagccttaaagacgctccttcagtgagcaaaaaaattttagcagaagctaaaaagctaaacgatgctcaagcaccaaaa

SEQ ID NO: 87 - DNA, 2667 bp, Artificial Sequence
  DNA encoding fusion protein
gatacacctcaaaaagatactacagctaagacaacatctcatgattcaaaaaaatctaatgacgatgaaacttctaaggatactacaagtaaagatattgataaagcagacaacaataatacaagtaaccaagacaataacgacaaaaaatttaaaactatagacgacagcacttcagactctaacaatatcattgattttatttataagaatttaccacaaaccaatataaaccaattgctaaccaaaaataaatacgatgataattactcattaacaactttaatccaaaacctattcaatttaaattcggatatttctgattacgaacaacctcgtaatggcgaaaagtcaacaaatgattcgaataaaaacagtgacaatagcatcaaaaatgacactgatacgcaatcatctaaacaagataaagcagacaatcaaaaagcacctaaatcaaacaatacaaaaccaagtacatctaataagcaaccaaattcgccaaagccaacacaacctaatcaatcaaatagtcaaccagcaagtgacgataaagcaaatcaaaaatcttcatcgaaagataatcaatcaatgtcagattcggctttagactctattttggatcaatacagtgaagatgcaaagaaaacacaaaaagattatgcatctcaatctaaaaaagacaaaaatgaaaaatctaatacaaagaatccacagttaccaacacaagatgaattgaaacataaatctaaacctgctcaatcattcaataacgatgttaatcaaaaggatacacgtgcaacatcattattcgaaacagatcctagtatatctaacaatgatgatagcggacaatttaacgttgttgactcaaaagatacacgtcaatttgtcaaatcaattgctaaagatgcacatcgcattggtcaagataacgatatttatgcgtctgtcatgattgcccaagcaatcttagaatctgactcaggtcgtagtgctttagctaagtcaccaaaccataatttattcggtatcaaaggtgcttttgaagggaattctgttccttttaacacattacaggctgatggtaataaattgtatagtattaatgctggattccgaaaatatccaagcacgaaagaatcactaaaaaactactctgaccttattaaaaatggtattgatggcaatcgaacaatttataaaccaacatggaaatcggaagccgattcttataaagatgcaacatcacacttatctaaaacatatgctacagatccaaactatgctaagaaattaaacagtattattaaacactatcaattaactcagtttgacgatgaacgcatgccagatttagataaatatgaacgttctatcaaggattatgatgattcatcagatgaattcaaacctttccgtgaggtatctgatagtatgccatatccacatggtcaaagcacttggtacgtatataaccgtatgaaacaatttggtacatctatctcaggtgatttaggtgatgcacataattggaataatcgagctcaataccgtgattatcaagtaagtcatacaccaaaacgtcatgctgctgttgtatttgaggctggacaatttggtgcagatcaacattacggtcatgtagcatttgttgaaaaagttaacagtgatggttctatcgttatttcagaatccaatgttaaaggattaggtatcatttctcatagaactatcaatgcagctgccgctgaagaattatcatatattacaggtaaaggttctggcggaggggctgcgcaagtggatgaagctcaaaaaaatgctttttatcaagtcttaaatatgcctaacttaaatgctgatcaacgcaatggttttatccaaagccttaaagatgctccaagccaaagtgctaaagttttaggtgaagctcaaaaacttaatgactctcaagctccaaaagctgatgcgcaacaaaataacgtgaacaaagatcaaaaaagcgccttctatgaaatcttgaacatgcctaacttaaacgaagcgcaacgtaacggcttcattcaaagtcttaaagacgctccaagccaaagcactaaagttttaggtgaagctaaaaaattaaacgaatctcaagcaccgaaagctgataacaatgtgaacaaagaacaaaaaaatgctttctatgaaatcttgaatatgcctaacttaaacgaagaacaacgcaatggtttcatccaaagcttaaaagatgctccaagccaaagtgctaaactattgtcagaagctaaaaagttaaatgaatctcaagcaccgaaagcggataacaaagtgaacaaagaacaaaaaaatgctttctatgaaatcttacatttacctaacttaaacgaagaacaacgcaatggtttcatccaaagcctaaaagatgctccaagccaaagcgctaaacttttagcagaagctaaaaagctaaatgatgctcaagcaccaaaagctgacaacaaagtgaacaaagaacaaaaaaatgctttctatgaaattttacatttacctaacttaactgaagaacaacgtaacggcttcatccaaagccttaaagacgctccttcagtgagcaaaaaaattttagcagaagctaaaaagctaaacgatgctcaagcaccaaaa

SEQ ID NO: 88 - DNA, 2118 bp, Artificial Sequence
  DNA encoding fusion protein
atggatattggtaaaaaacatgtaattcctaaaagtcagtaccgacgtaagcgtcgtgaattcttccacaacgaagacagagaagaaaatttaaatcaacatcaagataaacaaaatatagataatacaacatcaaaaaaagcagataagcaaatacataaagattcaattgataagcacgaacgttttaaaaatagtttatcatcgcatttagaacagagaaaccgtgatgttaatgagaataaagctgaagaaagtaaaagtaatcaggatagtaagtcagcatataacagagatcattatttaacagacgatgtatctaaaaaacaaaattcattagattcagtggaccaagatacagagaaatcaaaatattatgagcaaaattctgaagcgactttatcaactaaatcaaccgataaagtagaatcaactgaaatgagaaagctaagttcagataaaaacaaagttggtcatgaagagcaacatgtactttctaaaccttcagaacatgataaagagactagaattgattctgagtcttcaagaactgattcagacagctcgatgcagacagagaaaataaaaaaagacagttcagatggaaataaaagtagtaatctgaaatctgaagtaatatcagacaaatcaaatacagtaccaaaattgtcggaatctgatgatgaagtaaataatcagaagccattaactttaccggaagaacagaaattgaaaagacagcaaagtcaaaatgagcaaacaaaaacctatacatatggtgatagcgaacaaaatgacaagtctaatcatgaaaatgatttaagtcatcatataccatcgataagtgatgataaagataacgtcatgagagaaaatcatattgttgacgataatcctgataatgatatcaatacaccatcattatcaaaaacagatgacgatcgaaaacttgatgaaaaaattcatgttgaagataaacataaacaaaatgcagactcgtctgaaacggtgggatatcaaagtcagtcaactgcatctcatcgtagcactgaaaaaagaaatatttctattaatgaccatgataaattaaacggtcaaaaaacaaatacaaagacatcggcaaataataatcaaaaaaaggctacatcaaaattgaacaaagggcgcgctacgaataataattatagtgacattttgaaaaagttttggatgatgtattggcctaaaggttctggcggaggggctgcgcaagtggatgaagctcaaaaaaatgctttttatcaagtcttaaatatgcctaacttaaatgctgatcaacgcaatggttttatccaaagccttaaagatgctccaagccaaagtgctaaagttttaggtgaagctcaaaaacttaatgactctcaagctccaaaagctgatgcgcaacaaaataacgtgaacaaagatcaaaaaagcgccttctatgaaatcttgaacatgcctaacttaaacgaagcgcaacgtaacggcttcattcaaagtcttaaagacgctccaagccaaagcactaaagttttaggtgaagctaaaaaattaaacgaatctcaagcaccgaaagctgataacaatgtgaacaaagaacaaaaaaatgctttctatgaaatcttgaatatgcctaacttaaacgaagaacaacgcaatggtttcatccaaagcttaaaagatgctccaagccaaagtgctaaactattgtcagaagctaaaaagttaaatgaatctcaagcaccgaaagcggataacaaagtgaacaaagaacaaaaaaatgctttctatgaaatcttacatttacctaacttaaacgaagaacaacgcaatggtttcatccaaagcctaaaagatgctccaagccaaagcgctaaacttttagcagaagctaaaaagctaaatgatgctcaagcaccaaaagctgacaacaaagtgaacaaagaacaaaaaaatgctttctatgaaattttacatttacctaacttaactgaagaacaacgtaacggcttcatccaaagccttaaagacgctccttcagtgagcaaaaaaattttagcagaagctaaaaagctaaacgatgctcaagcaccaaaa

SEQ ID NO: 89 - DNA, 1740 bp, Artificial Sequence
  DNA encoding fusion protein
aatactaatattgaaaatattggtgatggtgctgaagtaatcaaacgtacggaggatgtaagtagtaagaaatggggcgttactcaaaatgtccaattcgactttgtaaaagataaaaaatataacaaagacgctttaattgttaaaatgcaaggttttattaattccagaacttcattttcagatgtgaagggtagtggatatgaattaactaaacgaatgatttggccattccaatataatataggactgacgactaaagatccaaatgttagcttaatcaattaccttcctaaaaacaaaatagaaactactgatgttggtcaaacattaggatataacattggaggtaatttccagtcagcaccatctataggtggcaatggctcatttaattattctaaaacaattagttatacccaaaagagttatgtcagtgaagtagacaagcaaaactcaaaatctgttaaatggggtgttaaagcaaacgaatttgttacgcctgatggaaaaaaatctgcgcatgatagatatttattcgtacaaagtccaaatggtccaacaggttcagcaagagaatattttgctcctgataatcaattgccacctttagttcaaagtggctttaatccatcgtttatcactacactatcacatgaaaaaggttcaagtgatacgagtgaatttgaaatttcatatggtagaaacttagatattacatatgcgactttattccctagaactggtatttacgcagaaagaaagcataatgcatttgtaaatagaaactttgtagttagatatgaagttaattggaaaacacacgaaattaaagtgaaaggacataatggttctggcggaggggctgcgcaagtggatgaagctcaaaaaaatgctttttatcaagtcttaaatatgcctaacttaaatgctgatcaacgcaatggttttatccaaagccttaaagatgcaccagcaaaaagtgctaacgttttaggtgaagctcaaaaacttaatgactctcaagctccaaaagctgatgcgcaacaaaataacgtgaacaaagatcaaaaaagcgccttctatgaaatcttgaacatgcctaacttaaacgaagcgcaacgtaacggcttcattcaaagtcttaaagacgcaccagcaaaaagcactaacgttttaggtgaagctaaaaaattaaacgaatctcaagcaccgaaagctgataacaatgtgaacaaagaacaaaaaaatgctttctatgaaatcttgaatatgcctaacttaaacgaagaacaacgcaatggtttcatccaaagcttaaaagatgcaccagcaaaaagtgctaacctattgtcagaagctaaaaagttaaatgaatctcaagcaccgaaagcggataacaaagtgaacaaagaacaaaaaaatgctttctatgaaatcttacatttacctaacttaaacgaagaacaacgcaatggtttcatccaaagcctaaaagatgcaccagcaaaaagcgctaaccttttagcagaagctaaaaagctaaatgatgctcaagcaccaaaagctgacaacaaagtgaacaaagaacaaaaaaatgctttctatgaaattttacatttacctaacttaactgaagaacaacgtaacggcttcatccaaagccttaaagacgcacctgcaaaaagcaaagaaattttagcagaagctaaaaagctaaacgatgctcaagcaccaaaa

(19) United States
(12) Patent Application Publication
(10) Pub. No.: US 2025/0288657 A1
(43) Pub. Date: Sep. 18, 2025

(54) Staphylococcal protein variants and truncates

(71) Applicant: Evaxion Biotech A/S, Hørsholm (DK)

(72) Inventors: Andreas Holm Mattsson, Hørsholm (DK); Jens Vindahl Kringelum, Hørsholm (DK); Pär Comstedt, Hørsholm (DK); Christian Thygesen, Hørsholm (DK)

(21) Appl. No.: 18/862,656

(22) PCT Filed: May 4, 2022
(86) PCT No.: PCT/EP2022/062056

§ 371 Date: Nov. 4, 2024

(51) Int. Cl. A61K 39/085; A61K 39/00; A61P 37/04; C07K 14/31; C12N 9/52; C12N 9/80

(52) U.S. Cl. CPC A61K 39/085; A61P 37/04; C07K 14/31; C12N 9/52; C12N 9/80; A61K 2039/545; A61K 2039/55505; A61K 2039/55555; A61K 2039/575; A61K 2039/627; A61K 2039/70; C12Y 304/2105; C12Y 304/24029; C12Y 305/01028

(57) ABSTRACT

Novel polypeptides, polynucleotides, expression vectors and novel immunogenic compositions derived from Staphylococcus aureus, in particular novel polypeptides, polynucleotides, expression vectors and compositions derived from/related to the SpA, Hla, Aur and LukE polypeptides. Also disclosed is methods of immunity induction utilising the polypeptides, polynucleotides, expression vectors, and immunogenic compositions.

### FIELD OF THE INVENTION

The present invention relates to the field of immunization technology, including vaccine technology. In particular, the present invention relates to novel variants of staphylococcal proteins as well as to compositions comprising staphylococcal proteins. The invention also relates to vectors and transformed cells and virus as well as compositions comprising these.

### BACKGROUND OF THE INVENTION

Staphylococcus aureus is a Gram-positive opportunistic pathogenic bacterium which is a major clinical challenge. Especially the multidrug-resistant Methicillin-resistant S. aureus (MRSA) strains, which give rise to serious hospital-acquired as well as community-acquired infections, are a great global health problem. There is a pressing need for alternatives to antibiotics for treatment of S. aureus infections and for a vaccine.

Despite many years of research, no vaccine against S. aureus has yet passed the test in clinical trials. The traditional strategy of developing a vaccine that elicits opsonizing antibodies against S. aureus surface proteins, leading to antibody-mediated clearance of the bacteria, has not been successful. A different and more promising strategy is to develop vaccines with the aim of neutralizing one or more of the virulence factors of S. aureus. The virulence factors of S. aureus include several different proteins with toxic and immune-evasive functions. Below is given a short description of the specific S. aureus virulence factors and other S. aureus proteins of the current disclosure.

### Immunoglobulin G-Binding Protein A

Immunoglobulin G-binding protein A (SpA) is a protein containing 4 to 5 homologous immunoglobulin binding domains (E, D, A, B, C), which each bind the constant region of IgG (Fcγ) and the Ig fragment (Fab) involved in antigen binding. The SpA Fc binding site can also bind to von Willebrand factor (vWF). SpA induces immune evasion by several mechanisms. The binding of SpA to the Fcγ domain interferes with the anti-microbial function of IgGs, including complement fixation and opsonophagocytosis. The binding to Fab provides SpA with a potent B cell super-antigen function, where secreted SpA crosslinks VH3-containing B cell receptors and triggers secretion of all VH3 antibodies, irrespective of their antigen specificity. As a result, antibody-mediated protective immunity against S. aureus is substantially impaired.

### Alpha-Hemolysin

Alpha-hemolysin (Hla), also known as alpha-toxin, is a member of the beta-barrel toxin family and is the major cytotoxic agent of S. aureus. Hla is a monomer, and seven copies of Hla self-assemble to form a heptameric pore in the cell membrane, which allows exchange of monovalent ions, leading to DNA fragmentation and apoptosis. The heptameric pore is composed of three structural regions: the cap, rim, and stem. Hla monomers may also oligomerize to form larger, Ca²⁺-permissive pores, which induce massive necrosis.

### HtrA2

HtrA-like proteases are involved in the virulence of both Gram-positive and Gram-negative bacteria. They are known to play a role in stress resistance and survival. In Streptococcus pyogenes, HtrA has been reported to intervene in the processing of an extracellular virulence factor and to play a role in the control of hemolytic activity. Two putative HtrA-like proteases, HtrA1 and HtrA2, are encoded by S. aureus. HtrA2 of S. aureus is classified as a transmembrane protein, and it contains a domain predicted to be an active serine protease with an Asp/His/Ser catalytic triad.

### LukE

The LukE protein is one of two components of the lukED leukocidin. In addition to inducing cell death in leukocytes, the pore-forming toxin LukED can lyse erythrocytes. The target cells (in humans and mice) of lukED include neutrophils, monocytes, macrophages, dendritic cells, T cells, erythrocytes and NK cells. LukE recognizes the receptors CCR5, CXCR1, and CXCR2 and the erythroid receptor DARC. No pores are formed by LukE alone, only by the heterodimer of LukE and LukD, after recognition of the cell surface receptors by LukE.

### Aureolysin

Aureolysin (Aur) is a zinc metalloproteinase. It is a S. aureus virulence factor with multiple roles, both in immune evasion and toxicity. Aur activates the glutamyl endopeptidase, which is a key virulence factor of S. aureus that cleaves certain human inflammatory regulators and immune components and inhibits the activation of components of the complement system. Aur directly cleaves complement C3 and inhibits the deposition of C3b on the bacterial surface and the release of the chemoattractant C5a. Furthermore, Aur activates prothrombin in human plasma and induces staphylocoagulation.

### SAR2753

Lipase 1/Glycerol ester hydrolase 1 (SAR2753) is a lipase which converts triacylglycerol+H₂O into diacylglycerol+a fatty acid+H⁺. SAR2753 may play a virulent role in S. aureus by its degradation of antimicrobial lipids produced by the host during infection.

### SAR2723

N-acetylmuramoyl-L-alanine amidase (SAR2723) is a protein containing the two amidase domains LYZ2/FlgJ and CHAP, which both have a main function in peptidoglycan hydrolysis. Consistently, many proteins comprising these domains are involved in cell wall metabolism and biogenesis.

### EsaA

The EsaA protein of S. aureus is a fundamental protein of the specialized type VII protein secretion system (T7SS) present in many Gram-positive bacteria. It is a membrane-spanning protein with 6 transmembrane domains. The S. aureus T7SS, termed T7b, consists of six essential proteins; EsxA, EssC, EsaA, EssA, Essb, and EsaB. T7b mediates the secretion of different proteins, including the toxins EsaC and EsaD, which contribute to S. aureus virulence. Furthermore, the T7b-mediated secretion contributes to the production or suppression of specific cytokines during host infection, thereby enabling S. aureus to manipulate immune responses.

### OBJECT OF THE INVENTION

It is an object of embodiments of the invention to provide immunogens and immunogenic compositions useful in vaccination against SA infection. It is further an object of the invention to provide methods of immunization/vaccination against SA infection that utilise these immunogens and immunogenic compositions.

### SUMMARY OF THE INVENTION

The present inventors have conducted a thorough research programme to identify novel pharmaceutically acceptable immunogens, which are derived from S. aureus antigens, and which are believed to be suitable for immunization/vaccination purposes. Part of the research has focused on reducing or eliminating the risk of administering immunogens that would be able to cause adverse effects akin to those exhibited by the wild-type antigen from which the immunogens are derived. Also, the present inventors have identified optimized vaccine compositions that are believed to induce useful immune responses against a variety of separate S. aureus antigens so as to effectively block various effector molecules of S. aureus.

Hence, in a first aspect, the present invention relates to an immunogenic polypeptide consisting of or comprising
i. a variant of the amino acid sequence of Immunoglobulin G-binding protein A (SpA), which variant comprises at least one of a-e:
   a. a sequence at least 85% identical to the amino acid sequence of Immunoglobulin binding domain (IgBD) E (SEQ ID NO: 16, residues 1-56),
   b. a sequence at least 85% identical to the amino acid sequence of IgBD D (SEQ ID NO: 16, residues 62-117),
   c. a sequence at least 85% identical to the amino acid sequence of IgBD A (SEQ ID NO: 16, residues 120-175),
   d. a sequence at least 85% identical to the amino acid sequence of IgBD B (SEQ ID NO: 16, residues 178-233),
   e. a sequence at least 85% identical to the amino acid sequence of IgBD C (SEQ ID NO: 16, residues 236-291),
   and
ii. one or more first mutation(s) in each of the at least one of a-e, wherein the one or more first mutation(s) disrupt(s) binding in the Fc-binding site and wherein the one or more first mutation(s) is/are made in positions corresponding to amino acid positions in SEQ ID NO: 16 selected from positions 3, 8, 9, 64, 69, 70, 122, 127, 128, 180, 185, 186, 238, 243, 244, and
iii. one or more second mutation(s) in each of the at least one of a-e, wherein the one or more second mutation(s) disrupt(s) binding in the Fab-binding site and wherein the one or more second mutation(s), where applicable, is/are made in positions corresponding to amino acid positions in SEQ ID NO: 16 selected from positions 34, 35, 37, 38, 41, 95, 96, 98, 99, 102, 153, 154, 156, 157, 160, 211, 212, 214, 215, 218, 269, 270, 272, 273, and 276,
iv. wherein, optionally, none of the at least one of a-e, where applicable, comprise a mutation, which disrupts binding in the Fc-binding site, in positions corresponding to both of amino acid positions 7+8, 68+69, 126+127, 184+185, and 242+243 of SEQ ID NO: 16, and
v. wherein none of the at least one of a-e, where applicable, comprise a mutation, which disrupts binding in the Fab-binding site, in positions corresponding to both of amino acid positions 34+35 and 95+96 and 153+154 and 211+212, and 269+270 of SEQ ID NO: 16,
   wherein the polypeptide is unable to bind to human IgG and human von Willebrand factor and wherein the substitution(s) are preferably non-conservative substitutions.

In a second aspect, the present invention relates to an immunogenic polypeptide consisting of or comprising a variant of the amino acid sequence of Alpha-hemolysin (Hla), which variant:
i. has at least 85% sequence identity with the sequence of SEQ ID NO: 2, and
ii. comprises one or more amino acid deletion(s) and/or substitution(s) in the first 12 consecutive N-terminal amino acid residues of the amino acid sequence of mature Hla, said first 12 consecutive N-terminal amino acid residues corresponding to positions 1 to 12 of SEQ ID NO: 2,
   wherein the polypeptide is unable to participate in formation of a heptameric structure with other Hla molecules and wherein any substitution(s) in ii) are preferably non-conservative.

In a third aspect, the present invention relates to an immunogenic polypeptide consisting of or comprising a variant of the amino acid sequence of LukE, which variant:
i. has at least 85% sequence identity with SEQ ID NO: 30, and
ii. does not comprise a signal peptide, which signal peptide corresponds to residues 1-28 of SEQ ID NO: 29.

In a fourth aspect, the present invention relates to an immunogenic polypeptide consisting of or comprising a variant of the amino acid sequence of Aureolysin (Aur), which variant:
i. has at least 85% sequence identity with the amino acid sequence of SEQ ID NO: 34, and
ii. comprises one or more amino acid substitutions in the HEXXH catalytic domain, where the HEXXH catalytic domain corresponds to amino acid positions 352-356 of SEQ ID NO: 34,
   wherein the polypeptide has reduced catalytic capacity and wherein the substitution(s) are preferably non-conservative.

In a fifth aspect, the present invention relates to an immunogenic polypeptide consisting of or comprising a variant of the amino acid sequence of N-acetylmuramoyl-L-alanine amidase (SAR 2723), which variant:
i. has at least 85% sequence identity with the amino acid sequence of SEQ ID NO: 38, and
   a. comprises one or more amino acid substitutions in the amidase active site TXEXX domain corresponding to amino acid residues 384-388 in SEQ ID NO: 37, and/or
   b. comprises one or more substitutions in the amidase active site LXDYX domain corresponding to amino acid residues 409-413 in SEQ ID NO: 37, and/or
   c. comprises a substitution of a conserved cysteine corresponding to position 513 in SEQ ID NO: 37,
      wherein the polypeptide has reduced catalytic capacity and wherein the substitution(s) are preferably non-conservative.

In a 6^{th} aspect, the present invention relates to a vaccine composition comprising a selection of polypeptides, which includes at least 2 of a-d:
a) an Hla polypeptide or a variant thereof, where said variant preferably exhibits reduced or no hemolytic activity and/or which preferably can induce antibodies that block the hemolytic activity of native Hla,
b) a LukE polypeptide or a variant thereof, where said variant preferably exhibits reduced or no leukocytic activity and/or which preferably can induce antibodies that block the leukocytic activity of native LukE,
c) an SpA polypeptide or a variant thereof, where said variant preferably exhibits a reduced ability to bind human IgG and human von Willebrandt Factor and/or which preferably can induce antibodies that block native SpA interaction with human IgG, and
d) an Aur polypeptide or a variant, thereof where said variant preferably exhibits reduced catalytic activity and which preferably can induce antibodies that block the catalytic activity of native Aur, said composition optionally further comprising a pharmaceutically acceptable carrier, vehicle or diluent, and further optionally an immunogenic adjuvant, which is in particular selected from AIOH, SLA-SE, and OMVs (outer membrane vesicles).

In a 7^{th} aspect, the present invention relates to a chimeric polypeptide comprising amino acid sequences of the selection of polypeptides according to the 6^{th} aspect of the invention, wherein the amino acid sequences are fused or connected via a linker.

In an 8^{th} aspect, the present invention relates to a nucleic acid fragment encoding the immunogenic polypeptide according to any one of the first to fifth aspects of the invention or the chimeric polypeptide according to the 7^{th} aspect of the invention, such as a DNA fragment or an RNA fragment.

In a 9^{th} aspect, the present invention relates to a vector comprising the nucleic acid fragment according to the 8^{th} aspect of the invention.

In a 10^{th} aspect, the present invention relates to a transformed cell or virus, which comprises and is capable of expressing the nucleic acid fragment according to the 8^{th} aspect of the invention or the vector according to the 9^{th} aspect of the invention.

In an 11^{th} aspect, the present invention relates to an immunogenic composition comprising the nucleic acid fragment according to the 8^{th} aspect of the invention, the vector according to embodiments of the 9^{th} aspect of the invention, or the transformed cell or virus according to the 10^{th} aspect of the invention and a pharmaceutically acceptable carrier, vehicle or diluent, and optionally an immunogenic adjuvant.

In a 12^{th} aspect, the present invention relates to an immunogenic composition comprising (a) nucleic acid fragment(s), vector(s) or transformed cell(s) or virus that is/are capable of expressing the selection of polypeptides according to the 6^{th} aspect of the invention, and a pharmaceutically acceptable carrier, vehicle or diluent, and optionally an immunogenic adjuvant.

In a 13^{th} aspect, the present invention relates to a method for inducing immunity in an animal by administering at least once an immunogenically effective amount of the immunogenic polypeptide according to embodiments of any one of the first to fifth aspect of the invention, a vaccine composition according to the 6^{th} aspect of the invention, a chimeric polypeptide according to the 7^{th} aspect of the invention, a nucleic acid fragment according to the 8^{th} aspect of the invention, a vector according to the 9^{th} aspect of the invention, a transformed cell or virus according to the 10^{th} aspect of the invention, or an immunogenic composition according to the 11^{th} or 12^{th} aspect of the invention, so as to induce adaptive immunity against S. aureus in the animal.

In a 14^{th} aspect, the present invention relates to an immunogenic polypeptide according to any one of the first to fifth aspects of the invention for use as a pharmaceutical.

In a 15^{th} aspect, the present invention relates to an immunogenic polypeptide according to any one of the first to fifth aspects of the invention for use as a pharmaceutical in the treatment, prophylaxis or amelioration of infection with S. aureus.

In a 16^{th} aspect, the present invention relates to a chimeric polypeptide according to the 7^{th} aspect of the invention for use as a pharmaceutical.

In a 17^{th} aspect, the present invention relates to a chimeric polypeptide according to the 7^{th} aspect of the invention for use as a pharmaceutical in the treatment, prophylaxis or amelioration of infection with S. aureus.

In an 18^{th} aspect, the present invention relates to a nucleic acid fragment according to the 8^{th} aspect of the invention or a vector according to the 9^{th} aspect of the invention for use as a pharmaceutical.

In a 19^{th} aspect, the present invention relates to a nucleic acid fragment according to the 8^{th} aspect of the invention or a vector according to the 9^{th} aspect of the invention for use as a pharmaceutical in the treatment, prophylaxis or amelioration of infection with S. aureus.

In a 20^{th} aspect, the present invention relates to a transformed cell or virus according to the 10^{th} aspect of the invention for use as a pharmaceutical.

In a 21^{st} aspect, the present invention relates to a transformed cell or virus according to the 10^{th} aspect of the invention for use as a pharmaceutical in the treatment, prophylaxis or amelioration of infection with S. aureus.

### LEGENDS TO THE FIGURES

FIG. 1: Non-specific binding of different SpA variants to mouse IgG, as determined by direct ELISA (see Example 1.1).

FIG. 2: Non-specific binding of different SpA variants to human IgG, as determined by direct ELISA (see Example 1.1).

FIG. 3: Non-specific binding of different SpA variants to mouse serum IgG, as determined by direct ELISA (see Example 1.1).

FIG. 4: Non-specific binding of different SpA variants to mouse serum IgM, as determined by direct ELISA (see Example 1.1).

FIG. 5: Non-specific binding of different SpA variants to rabbit serum IgG, as determined by direct ELISA (see Example 1.1).

FIG. 6: Binding of different SpA variants to vWF, as determined by direct ELISA. SpA_WT-49-339 binding was set as 100% and relative SpA binding (in percentage) was calculated based on this value (see Example 1.1).

FIG. 7: Non-specific binding of different SpA variants to human IgG, as determined by competitive ELISA (see Example 1.1).

FIG. 8: A-B: Ability of immune sera from mice vaccinated with different single and chimeric proteins to inhibit the activity of aureolysin, as determined by an azocasein assay (see Example 1.2).

FIG. 9: Ability of immune sera from mice vaccinated with LukE alone or chimeric proteins including LukE to inhibit the leukocytic activity of LukED, as determined by an XTT assay (see Example 1.3).

FIG. 10: Ability of immune sera from mice vaccinated with Hla alone or chimeric proteins including Hla to inhibit the hemolytic activity of Hla, as determined by an erythrocyte hemolysis assay (see Example 1.4).

FIG. 11: Immunogenicity, Half-max IgG data from mice immunized with single or chimeric proteins adjuvanted with SLA-SE (see Example 4).

FIG. 12: Immunogenicity, Half-max IgG data from mice immunized with single or chimeric proteins adjuvanted with OMV+AIOH or AIOH (see Example 4).

### DETAILED DISCLOSURE OF THE INVENTION

### Definitions

The term “polypeptide” is in the present context intended to mean both short peptides of from 2 to 10 amino acid residues, oligopeptides of from 11 to 100 amino acid residues, and polypeptides of more than 100 amino acid residues. Further-more, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring amino acid sequence or nucleic acid sequence, respectively

The term “amino acid sequence” is the order in which amino acid residues, connected by peptide bonds, lie in the chain in peptides and proteins when listed in the direction from the N- to the C-terminus.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Sequence identity” is in the context of the present invention determined by comparing 2 optimally aligned sequences of equal length (e.g. DNA, RNA or amino acid) according to the following formula: (N_{ref}−N_{dif})·100/N_{ref}, wherein N_{ref} is the number of residues in one of the 2 sequences and N_{dif} is the number of residues which are non-identical in the two sequences when they are aligned over their entire lengths and in the same direction. So, two sequences 5′-ATTCGGAAC-3′ and 5′-ATACGGGAC-3′ will provide the sequence identity 77.8% (N_{ref}=9 and N_{dif}=2).

The “3D conformation” is the 3-dimensional structure of a biomolecule such as a protein. In monomeric polypeptides/proteins, the 3D conformation is also termed “the tertiary structure” and denotes the relative locations in 3-dimensional space of the amino acid residues forming the polypeptide.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right-typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule prese

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

An “adaptive immune response” is an immune response in response to confrontation with an antigen or immunogen, where the immune response is specific for antigen determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention substantially all will mount an immune response against the immunogen of the present invention.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

“Specific binding” denotes binding between two substances which goes beyond binding of either substance to randomly chosen substances and also goes beyond simple association between substances that tend to aggregate because they share the same overall hydrophobicity or hydrophilicity. As such, specific binding usually involves a combination of electrostatic and other interactions between two conformationally complementary areas on the two substances, meaning that the substances can “recognize” each other in a complex mixture.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage-both these infectious agents are capable of introducing a heterologous nucleic acid sequence into a host cell.

The term “expression vector” refers to a vector containing a nucleic acid sequence coding for at least part of a gene product capable of being transcribed. In some cases, when the transcription product is an mRNA molecule, this is in turn translated into a protein, polypeptide, or peptide.

When referring to an “amino acid”, the present disclosure in general refers to proteinogenic amino acids, i.e. amino acids that are encoded by nucleic acids and appear in expression products for such nucleic acids, as well as to non-proteinogenic amino acids. However, the 22 naturally occurring amino acids are preferred amino acids appearing in the polypeptides disclosed herein, since such polypeptides can be recombinantly produced.

The amino acid substitutions mentioned in the following table are considered “conservative”. Substitutions not mentioned in the table are consequently considered “non-conservative”.

| Amino acid | Conservative mutations |
|---|---|
| Alanine (A) | C, G, S, T |
| Arginine (R) | K, H |
| Asparagine (N) | D, S, T, Q, E |
| Aspartic acid (D) | E, N, S, T, Q |
| Cysteine (C) | — |
| Glutamic acid (E) | D, Q, N, S, T |
| Glutamine (Q) | E, S, T, N, D |
| Glycine (G) | C, A, S, T |
| Histidine (H) | — |
| Isoleucine (I) | M, L, V |
| Leucine (L) | M, I, V |
| Lysine (K) | R, H |
| Phenylalanine (F) | Y, W, H |
| Methionine (M) | L, I, V |
| Proline (P) | — |
| Serine (S) | T, C, G, A |
| Threonine (T) | S, C, G, A |
| Tryptophan (W) | F, Y, H |
| Tyrosine (Y) | F, W, H |
| Valine (V) | M, I, L |

When variants of S. aureus derived polypeptides are mutated as discussed herein, one embodiment entails that the amino acids are non-conservative, but the most important substitutions are those that do not result in dramatic changes in secondary and tertiary structure of the substituted protein compared to the native protein. For instance, proline introduces a fixed turn or bend in a protein's secondary structure, so substitutions with proline will normally be avoided in order to not provide for polypeptides that are less likely to induce antibodies that would target the non-substituted protein. In general, the mutations preferred herein are those that render the mutated molecule less capable of exerting its pathological function (such as the IgG and von Willebrand binding activities of SpA), but which on the other hand preserves the secondary and tertiary structure of the unmutated protein or protein fragment, thereby enabling induction of non-mutated protein binging antibodies by the mutated variant. For instance, exchange of a non-polar amino acid residue with a polar amino acid residue or vice versa will generally be effective in interfering with the binding capability of the mutated variant even though the 3D shape of the molecule remains virtually unaltered.

When referring to an amino acid position or amino residue in a variant sequence or a homologous sequence, where the position or residue is said to “correspond to” an amino acid position in a reference amino acid sequence, it is herein intended that the reference sequence is optimally aligned with that of the homologous/variant sequence; when this is done, the “corresponding” residue/position is the one aligned with the residue pointed out in the reference sequence. Optimal alignment of two sequences must be made by preparing a global alignment of the reference and the variant/homologous sequences using the EMBOSS Needle alignment, for which an online tool is available at www.ebi.ac.uk/tools/psa (based on the Needleman-Wunsch algorithm), with the following settings for protein sequence alignments:
Matrix: Blosum62;
Gap open: 10;
Gap extend: 0.5;
End Gap Penalty: False;
End Gap Open: 10; and
End Gap Extend: 0.5.

When referring “sequence identity between amino acid sequences, the calculation is made using the same alignment. Thus, optimal alignment of two sequences must be made by preparing a global alignment of the reference and the variant/homologous sequences using the EMBOSS Needle alignment with the parameters set forth above in order to obtain a sequence identity percentage.

### SPECIFIC EMBODIMENTS OF THE INVENTION

### Embodiments of the 1^{st} Aspect of the Invention

The first aspect of the invention relates to an immunogenic polypeptide consisting of or comprising
i. a variant of the amino acid sequence of Immunoglobulin G-binding protein A (SpA), which variant comprises at least one of a-e:
   a. a sequence at least 85% identical to the amino acid sequence of Immunoglobulin binding domain (IgBD) E (SEQ ID NO: 16, residues 1-56),
   b. a sequence at least 85% identical to the amino acid sequence of IgBD D (SEQ ID NO: 16, residues 62-117),
   c. a sequence at least 85% identical to the amino acid sequence of IgBD A (SEQ ID NO: 16, residues 120-175),
   d. a sequence at least 85% identical to the amino acid sequence of IgBD B (SEQ ID NO: 16, residues 178-233),
   e. a sequence at least 85% identical to the amino acid sequence of IgBD C (SEQ ID NO: 16, residues 236-291),
   and
ii. one or more first mutation(s) in each of the at least one of a-e, wherein the one or more first mutation(s) disrupt(s) binding in the Fc-binding site and wherein the one or more first mutation(s) is/are made in positions corresponding to amino acid positions in SEQ ID NO: 16 selected from positions 3, 8, 9, 64, 69, 70, 122, 127, 128, 180, 185, 186, 238, 243, 244, and
iii. one or more second mutation(s) in each of the at least one of a-e, wherein the one or more second mutation(s) disrupt(s) binding in the Fab-binding site and wherein the one or more second mutation(s), where applicable, is/are made in positions corresponding to amino acid positions in SEQ ID NO: 16 selected from positions 34, 35, 37, 38, 41, 95, 96, 98, 99, 102, 153, 154, 156, 157, 160, 211, 212, 214, 215, 218, 269, 270, 272, 273, and 276,
iv. wherein, optionally, none of the at least one of a-e, where applicable, comprise a mutation, which disrupts binding in the Fc-binding site, in positions corresponding to both of amino acid positions 7+8, 68+69, 126+127, 184+185, and 242+243 of SEQ ID NO: 16, and
v. wherein none of the at least one of a-e, where applicable, comprise a mutation, which disrupts binding in the Fab-binding site, in positions corresponding to both of amino acid positions 34+35 and 95+96 and 153+154 and 211+212, and 269+270 of SEQ ID NO: 16,
   wherein the polypeptide is unable to bind to human IgG and human von Willebrand factor and wherein the substitution(s) are preferably non-conservative substitutions

The specific mutation pattern in these novel variants of SpA have been demonstrated by the inventors to accomplish the combined goal of being immunogenic and being capable of inducing antibodies that bind the wild-type protein while at the same time not exhibiting the effects on IgG and von Willebrand factor, which are characteristic of wild-type SpA.

In a first embodiment of the first aspect of the invention, the one or more second mutation(s) is/are made in at least or only
1 or 2 or 3 or 4 or 5 of positions 34 and 96 and 154 and 212 and 270,
1 or 2 or 3 or 4 or 5 of positions 35 and 95 and 154 and 212 and 270,
1 or 2 or 3 or 4 or 5 of positions 35 and 96 and 153 and 212 and 270,
1 or 2 or 3 or 4 or 5 of positions 35 and 96 and 154 and 211 and 270,
1 or 2 or 3 or 4 or 5 of positions 35 and 96 and 154 and 212 and 269,
1 or 2 or 3 or 4 or 5 of positions 34 and 95 and 154 and 212 and 270,
1 or 2 or 3 or 4 or 5 of positions 34 and 96 and 153 and 212 and 270,
1 or 2 or 3 or 4 or 5 of positions 34 and 96 and 154 and 211 and 270,
1 or 2 or 3 or 4 or 5 of positions 34 and 96 and 154 and 212 and 269,
1 or 2 or 3 or 4 or 5 of positions 35 and 95 and 153 and 212 and 270,
1 or 2 or 3 or 4 or 5 of positions 35 and 95 and 154 and 211 and 270,
1 or 2 or 3 or 4 or 5 of positions 35 and 95 and 154 and 212 and 269,
1 or 2 or 3 or 4 or 5 of positions 35 and 96 and 153 and 211 and 270,
1 or 2 or 3 or 4 or 5 of positions 35 and 96 and 153 and 212 and 269,
1 or 2 or 3 or 4 or 5 of positions 35 and 96 and 154 and 211 and 269,
1 or 2 or 3 or 4 or 5 of positions 34 and 95 and 153 and 212 and 270,
1 or 2 or 3 or 4 or 5 of positions 34 and 95 and 154 and 211 and 270,
1 or 2 or 3 or 4 or 5 of positions 34 and 95 and 154 and 212 and 269,
1 or 2 or 3 or 4 or 5 of positions 34 and 96 and 153 and 211 and 270,
1 or 2 or 3 or 4 or 5 of positions 34 and 96 and 153 and 212 and 269,
1 or 2 or 3 or 4 or 5 of positions 34 and 96 and 154 and 211 and 269,
1 or 2 or 3 or 4 or 5 of positions 35 and 95 and 153 and 211 and 270,
1 or 2 or 3 or 4 or 5 of positions 35 and 95 and 153 and 212 and 269,
1 or 2 or 3 or 4 or 5 of positions 35 and 96 and 153 and 211 and 269,
1 or 2 or 3 or 4 or 5 of positions 34 and 95 and 153 and 211 and 270,
1 or 2 or 3 or 4 or 5 of positions 34 and 95 and 153 and 212 and 269,
1 or 2 or 3 or 4 or 5 of positions 34 and 95 and 154 and 211 and 269,
1 or 2 or 3 or 4 or 5 of positions 34 and 96 and 153 and 211 and 269,
1 or 2 or 3 or 4 or 5 of positions 35 and 95 and 153 and 211 and 269,
1 or 2 or 3 or 4 or 5 of positions 35 and 95 and 154 and 211 and 269,
1 or 2 or 3 or 4 or 5 of positions 34 and 95 and 153 and 211 and 269, or
1 or 2 or 3 or 4 or 5 of positions 35 and 96 and 154 and 212 and 270.

In a second embodiment of the first aspect of the invention, the one or more second mutation(s) is/are made at least or only in a position corresponding to a position in SEQ ID NO: 16 selected from the group consisting of 34, 35, 95, 96, 153, 154, 211, 212, 269, and 270.

In a third embodiment of the first aspect of the invention, the one or more second mutation(s) is/are made in a position corresponding to a position in SEQ ID NO: 16 selected from the group consisting of 34, 35, 95, 96, 153, 154, 211, 212, 269, and 270.

In a fourth embodiment of the first aspect of the invention, the one or more second mutation(s) are made at least or only in positions corresponding to positions in SEQ ID NO: 16 selected from the group consisting of 34 and 95, 34 and 96, 34 and 153, 34 and 154, 34 and 211, 34 and 212, 34 and 269, 34 and 270, 35 and 95, 35 and 96, 35 and 153, 35 and 154, 35 and 211, 35 and 212, 35 and 269, 35 and 270, 95 and 153, 95 and 154, 95 and 211, 95 and 212, 95 and 269, 95 and 270, 96 and 153, 96 and 154, 96 and 211, 96 and 212, 96 and 269, 96 and 270, 153 and 211, 153 and 212, 153 and 269, 153 and 270, 154 and 211, 154 and 212, 154 and 269, 154 and 270, 211 and 269, 211 and 270, 212 and 269, and 212 and 270.

In a fifth embodiment of the first aspect of the invention, the one or more second mutation(s) are made at least or only in positions corresponding to positions in SEQ ID NO: 16 selected from the group consisting of 34 and 95 and 153, 34 and 95 and 154, 34 and 95 and 211, 34 and 95 and 212, 34 and 95 and 269, 34 and 95 and 270, 35 and 95 and 153, 35 and 95 and 154, 35 and 95 and 211, 35 and 95 and 212, 35 and 95 and 269, 35 and 95 and 270, 34 and 96 and 153, 34 and 96 and 154, 34 and 96 and 211, 34 and 96 and 212, 34 and 96 and 269, 34 and 96 and 270, 35 and 96 and 153, 35 and 96 and 154, 35 and 96 and 211, 35 and 96 and 212, 35 and 96 and 269, 35 and 96 and 270, 34 and 153 and 211, 34 and 153 and 212, 34 and 154 and 269, 34 and 154 and 270, 35 and 153 and 211, 35 and 153 and 212, 35 and 154 and 269, 35 and 154 and 270, 34 and 211 and 269, 34 and 212 and 270, 35 and 211 and 269, 35 and 212 and 270, 95 and 153 and 211, 95 and 153 and 212, 95 and 153 and 269, 95 and 153 and 270, 95 and 154 and 211, 95 and 154 and 212, 95 and 154 and 269, 95 and 154 and 270, 96 and 153 and 211, 96 and 153 and 212, 96 and 153 and 269, 96 and 153 and 270, 96 and 154 and 211, 96 and 154 and 212, 96 and 154 and 269, 96 and 154 and 270, 95 and 211 and 269, 95 and 211 and 270, 95 and 212 and 269, 95 and 212 and 270, 96 and 212 and 269, 96 and 212 and 270, 96 and 212 and 269, 96 and 212 and 270, 153 and 211 and 269, 153 and 212 and 269, 153 and 211 and 270, 153 and 212 and 270, 154 and 211 and 269, 154 and 212 and 269, 154 and 211 and 270, and 154 and 212 and 270.

In a 6^{th} embodiment of the first aspect of the invention, the one or more second mutation(s) are made at least or only in positions corresponding to positions in SEQ ID NO: 16 selected from the group consisting of 95 and 153 and 211 and 269, 95 and 153 and 211 and 270, 95 and 153 and 212 and 269, 95 and 153 and 212 and 270, 95 and 154 and 211 and 269, 95 and 154 and 211 and 270, 95 and 154 and 212 and 269, 95 and 154 and 212 and 270, 96 and 153 and 211 and 269, 96 and 153 and 211 and 270, 96 and 153 and 212 and 269, 96 and 153 and 212 and 270, 96 and 154 and 211 and 269, 96 and 154 and 211 and 270, 96 and 154 and 212 and 269, 96 and 154 and 212 and 270, 34 and 153 and 211 and 269, 34 and 153 and 211 and 270, 34 and 153 and 212 and 269, 34 and 153 and 212 and 270, 34 and 154 and 211 and 269, 34 and 154 and 211 and 270, 34 and 154 and 212 and 269, 34 and 154 and 212 and 270, 35 and 153 and 211 and 269, 35 and 153 and 211 and 270, 35 and 153 and 212 and 269, 35 and 153 and 212 and 270, 35 and 154 and 211 and 269, 35 and 154 and 211 and 270, 35 and 154 and 212 and 269, 35 and 154 and 212 and 270, 34 and 95 and 211 and 269, 34 and 95 and 211 and 270, 34 and 95 and 212 and 269, 34 and 95 and 212 and 270, 34 and 96 and 211 and 269, 34 and 96 and 211 and 270, 34 and 96 and 212 and 269, 34 and 96 and 212 and 270, 35 and 95 and 211 and 269, 35 and 95 and 211 and 270, 35 and 95 and 212 and 269, 35 and 95 and 212 and 270, 35 and 96 and 211 and 269, 35 and 96 and 211 and 270, 35 and 96 and 212 and 269, 35 and 96 and 212 and 270, 34 and 95 and 153 and 269, 34 and 95 and 153 and 270, 34 and 95 and 154 and 269, 34 and 95 and 154 and 270, 34 and 96 and 153 and 269, 34 and 96 and 153 and 270, 34 and 96 and 154 and 269, 34 and 96 and 154 and 270, 35 and 95 and 153 and 269, 35 and 95 and 153 and 270, 35 and 95 and 154 and 269, 35 and 95 and 154 and 270, 35 and 96 and 153 and 269, 35 and 96 and 153 and 270, 35 and 96 and 154 and 269, 35 and 96 and 154 and 270, 34 and 95 and 153 and 211, 34 and 95 and 153 and 212, 34 and 95 and 154 and 211, 34 and 95 and 154 and 212, 34 and 96 and 153 and 211, 34 and 96 and 153 and 212, 34 and 96 and 154 and 211, 34 and 96 and 154 and 212, 35 and 95 and 153 and 211, 35 and 95 and 153 and 212, 35 and 95 and 154 and 211, 35 and 95 and 154 and 212, 35 and 96 and 153 and 211, 35 and 96 and 153 and 212, 35 and 96 and 154 and 211, and 35 and 96 and 154 and 212.

In a 7^{th} embodiment of the first aspect of the invention, the one or more second mutation(s) are made at least or only in positions corresponding to positions in SEQ ID NO: 16 selected from the group consisting of 34 and 96 and 154 and 212 and 270, 35 and 95 and 154 and 212 and 270, 35 and 96 and 153 and 212 and 270, 35 and 96 and 154 and 211 and 270, 35 and 96 and 154 and 212 and 269, 34 and 95 and 154 and 212 and 270, 34 and 96 and 153 and 212 and 270, 34 and 96 and 154 and 211 and 270, 34 and 96 and 154 and 212 and 269, 35 and 95 and 153 and 212 and 270, 35 and 95 and 154 and 211 and 270, 35 and 95 and 154 and 212 and 269, 35 and 96 and 153 and 211 and 270, 35 and 96 and 153 and 212 and 269, 35 and 96 and 154 and 211 and 269, 34 and 95 and 153 and 212 and 270, 34 and 95 and 154 and 211 and 270, 34 and 95 and 154 and 212 and 269, 34 and 96 and 153 and 211 and 270, 34 and 96 and 153 and 212 and 269, 34 and 96 and 154 and 211 and 269, 35 and 95 and 153 and 211 and 270, 35 and 95 and 153 and 212 and 269, 35 and 96 and 153 and 211 and 269, 34 and 95 and 153 and 211 and 270, 34 and 95 and 153 and 212 and 269, 34 and 95 and 154 and 211 and 269, 34 and 96 and 153 and 211 and 269, 35 and 95 and 153 and 211 and 269, 35 and 95 and 154 and 211 and 269, 34 and 95 and 153 and 211 and 269, and 35 and 96 and 154 and 212 and 270.

In an 8^{th} embodiment of the first aspect of the invention, the one or more first mutation(s) is/are made at least or only in a position corresponding to a position in SEQ ID NO: 16 selected from 8, 69, 127, 185, and 243.

In a 9^{th} embodiment of the first aspect of the invention, the one or more first mutation(s) are made in positions corresponding to positions in SEQ ID NO: 16 selected from 3 and 8; 3 and 9; 3 and 69; 3 and 127; 3 and 185; 3 and 243; 8 and 9; 8 and 69; 8 and 127; 8 and 185; 8 and 243; 9 and 69; 9 and 127; 9 and 185; 9 and 243; 69 and 127; 69 and 185; 69 and 243; 127 and 185; 127 and 243; 185 and 243; 3 and 8 and 9; 3 and 8 and 69; 3 and 8 and 127; 3 and 8 and 185; 3 and 8 and 243; 3 and 9 and 69; 3 and 9 and 127; 3 and 9 and 185; 3 and 9 and 243; 3 and 69 and 127; 3 and 69 and 185; 3 and 69 and 243; 3 and 127 and 185; 3 and 127 and 243; 3 and 185 and 243; 8 and 9 and 69; 8 and 9 and 127; 8 and 9 and 185; 8 and 9 and 243; 8 and 69 and 127; 8 and 69 and 185; 8 and 69 and 243; 8 and 127 and 185; 8 and 127 and 243; 8 and 185 and 243; 9 and 69 and 127; 9 and 69 and 185; 9 and 69 and 243; 9 and 127 and 185; 9 and 127 and 243; 9 and 185 and 243; 69 and 127 and 185; 69 and 127 and 243; 69 and 185 and 243; 127 and 185 and 243; 3 and 8 and 9 and 69; 3 and 8 and 9 and 127; 3 and 8 and 9 and 185; 3 and 8 and 9 and 243; 3 and 8 and 69 and 127; 3 and 8 and 69 and 185; 3 and 8 and 69 and 243; 3 and 8 and 127 and 185; 3 and 8 and 127 and 243; 3 and 8 and 185 and 243; 3 and 9 and 69 and 127; 3 and 9 and 69 and 185; 3 and 9 and 69 and 243; 3 and 9 and 127 and 185; 3 and 9 and 127 and 243; 3 and 9 and 185 and 243; 3 and 69 and 127 and 185; 3 and 69 and 127 and 243; 3 and 69 and 185 and 243; 3 and 127 and 185 and 243; 8 and 9 and 69 and 127; 8 and 9 and 69 and 185; 8 and 9 and 69 and 243; 8 and 9 and 127 and 185; 8 and 9 and 127 and 243; 8 and 9 and 185 and 243; 8 and 69 and 127 and 185; 8 and 69 and 127 and 243; 8 and 69 and 185 and 243; 8 and 127 and 185 and 243; 9 and 69 and 127 and 185; 9 and 69 and 127 and 243; 9 and 69 and 185 and 243; 9 and 127 and 185 and 243; 69 and 127 and 185 and 243; 3 and 8 and 9 and 69 and 127; 3 and 8 and 9 and 69 and 185; 3 and 8 and 9 and 69 and 243; 3 and 8 and 9 and 127 and 185; 3 and 8 and 9 and 127 and 243; 3 and 8 and 9 and 185 and 243; 3 and 8 and 69 and 127 and 185; 3 and 8 and 69 and 127 and 243; 3 and 8 and 69 and 185 and 243; 3 and 8 and 127 and 185 and 243; 3 and 9 and 69 and 127 and 185; 3 and 9 and 69 and 127 and 243; 3 and 9 and 69 and 185 and 243; 3 and 9 and 127 and 185 and 243; 3 and 69 and 127 and 185 and 243; 8 and 9 and 69 and 127 and 185; 8 and 9 and 69 and 127 and 243; 8 and 9 and 69 and 185 and 243; 8 and 9 and 127 and 185 and 243; 8 and 69 and 127 and 185 and 243; 9 and 69 and 127 and 185 and 243; 3 and 8 and 9 and 69 and 127 and 185; 3 and 8 and 9 and 69 and 127 and 243; 3 and 8 and 9 and 69 and 185 and 243; 3 and 8 and 9 and 127 and 185 and 243; 3 and 8 and 69 and 127 and 185 and 243; 3 and 9 and 69 and 127 and 185 and 243; 8 and 9 and 69 and 127 and 185 and 243; and 3 and 8 and 9 and 69 and 127 and 185 and 243.

In a 10^{th} embodiment of the first aspect of the invention, the first and/or second mutation(s) is/are non-conservative substitution(s). As pointed out above, such non-conservative substitutions are however not those that will interfere with secondary structure and tertiary structure. Put differently, the mutations should preferably preserve structure (to enable induction of effective antibodies that bind the non-mutated protein) but on the other hand the mutations should preferably reduce or abolish undesired functionality exhibited by the non-mutated protein. This also has the consequence that mutations that would conventionally be termed “conservative” are useful if they have the effect of rendering the immunogen less biologically active than the wild-type protein.

In an 11^{th} embodiment of the first aspect of the invention, at least one of the one or more first mutation(s) is/are as in the 8^{th} embodiment of the first aspect of the invention, and at least one of the one or more first mutation(s), which is/are as in the 8^{th} embodiment of the first aspect of the invention, is a mutation to lysine (K).

In a 12^{th} embodiment of the first aspect of the invention, at least one of the one or more second mutation(s) is/are as in the 8^{th} embodiment of the first aspect of the invention, and all of the one or more first mutation(s), which is/are as in the 8^{th} embodiment of the first aspect of the invention, are a mutation to lysine (K).

In a 13^{th} embodiment of the first aspect of the invention, at least one of the one or more second mutation(s) is/are as in the 3^{rd} embodiment of the first aspect of the invention, and at least one of the one or more second mutation(s), which is/are as in the 3^{rd} embodiment of the first aspect of the invention, is a mutation to alanine (A) or arginine (R).

In a 14^{th} embodiment of the first aspect of the invention, at least one of the one or more second mutation(s) is/are as in the 3^{rd} embodiment of the first aspect of the invention, and all of the one or more second mutation(s), which is/are as in the 3^{rd} embodiment of the first aspect of the invention, are a mutation to alanine (A) or arginine (R).

In a 15^{th} embodiment of the first aspect of the invention, the first mutation is according to the 11^{th} embodiment of the first aspect of the invention and the second mutation is according to the 13^{th} embodiment of the first aspect of the invention, or the first mutation is according to the 12^{th} embodiment of the first aspect of the invention and the second mutation is according to the 13^{th} embodiment of the first aspect of the invention, or the first mutation is according to the 11^{th} embodiment of the first aspect of the invention and the second mutation is according to the 14^{th} embodiment of the first aspect of the invention, or the first mutation is according to the 12^{th} embodiment of the first aspect of the invention and the second mutation is according to the 14^{th} embodiment of the first aspect of the invention.

In a 16^{th} embodiment of the first aspect of the invention, the variant has at least 85% sequence identity with the sequence of SEQ ID NO: 16.

In a 17^{th} embodiment of the first aspect of the invention, the polypeptide can induce antibodies that block the ability of wildtype SpA to bind to IgG and von Willebrand factor.

### Embodiments of the 2^{nd} Aspect of the Invention

The second aspect of the present invention relates to an immunogenic polypeptide consisting of or comprising a variant of the amino acid sequence of Alpha-hemolysin (Hla), which variant:
i. has at least 85% sequence identity with the sequence of SEQ ID NO: 2, and
ii. comprises one or more amino acid deletion(s) and/or substitution(s) in the first 12 consecutive N-terminal amino acid residues of the amino acid sequence of mature Hla, said first 12 consecutive N-terminal amino acid residues corresponding to positions 1 to 12 of SEQ ID NO: 2,
   wherein the polypeptide is unable to participate in formation of a heptameric structure with other Hla molecules and wherein any substitution(s) in ii) are preferably non-conservative.

As is the case with the polypeptide of the first aspect of the invention, the polypeptide of the 2^{nd} aspect has, due to the carefully selected changes in its primary structure relative to the wild-type protein (Hla), the ability to induce antibodies that bind Hla while at the same time exhibiting reduced biological reactivity.

In a first embodiment of the second aspect of the invention, the variant comprises at least two amino acid alterations selected from deletions and substitutions in said first 12 consecutive N-terminal amino acid residues, such as at least or exactly 2, at least or exactly 3, at least or exactly 4, at least or exactly 5, at least or exactly 6, at least or exactly 7, at least or exactly 8, at least or exactly 9, at least or exactly 10, at least or exactly 11, or exactly 12 amino acid alterations.

In a second embodiment of the second aspect of the invention, the variant comprises at least two amino acid substitutions in the first 12 N-terminal amino acids under ii), such as at least or exactly 2, at least or exactly 3, at least or exactly 4, at least or exactly 5, at least or exactly 6, at least or exactly 7, at least or exactly 8, at least or exactly 9, at least or exactly 10, at least or exactly 11, or exactly 12 amino acid substitutions.

In a third embodiment of the second aspect of the invention, the variant comprises at least two amino acid deletions in said first 12 consecutive N-terminal amino acid residues, such as at least or exactly 2, at least or exactly 3, at least or exactly 4, at least or exactly 5, at least or exactly 6, at least or exactly 7, at least or exactly 8, at least or exactly 9, at least or exactly 10, at least or exactly 11, or exactly 12 amino acid deletions.

In a fourth embodiment of the second aspect of the invention, amino acid residues 1-12 are all deleted.

In a fifth embodiment of the second aspect of the invention, amino acid residues 1-12 are all substituted.

In a 6^{th} embodiment of the second aspect of the invention, amino acid residues 1-12 are substituted with the sequence 5′-SETEVSVRSASS-3′ (residues 1-12 in SEQ ID NO: 4).

In a 7^{th} embodiment of the second aspect of the invention, the variant comprises SEQ ID NO: 3.

In an 8^{th} embodiment of the second aspect of the invention, the immunogenic polypeptide comprises a substitution from histidine (H) to leucine (L) at the position corresponding to position 35 of SEQ ID NO: 2.

In a 9^{th} embodiment of the second aspect of the invention, the variant comprises SEQ ID NO: 6.

In a 10^{th} embodiment of the second aspect of the invention, the variant lacks or has significantly reduced hemolytic activity.

In an 11^{th} embodiment of the second aspect of the invention, the polypeptide can induce antibodies that block the hemolytic activity of wildtype Hla.

### Embodiments of the 3^{rd} Aspect of the Invention

The third aspect of the present invention relates to an immunogenic polypeptide consisting of or comprising a variant of the amino acid sequence of LukE, which variant:
i. has at least 85% sequence identity with SEQ ID NO: 30, and
ii. does not comprise a signal peptide, which signal peptide corresponds to residues 1-28 of SEQ ID NO: 29.

In a first embodiment of the third aspect of the invention, the polypeptide consists of SEQ ID NO: 30.

In a second embodiment of the third aspect of the invention, the polypeptide can induce antibodies that block leukotoxicity of the functional mature wildtype LukE.

### Embodiments of the 4^{th} Aspect of the Invention

The fourth aspect of the present invention relates to an immunogenic polypeptide consisting of or comprising a variant of the amino acid sequence of Aureolysin (Aur), which variant:
i. has at least 85% sequence identity with the amino acid sequence of SEQ ID NO: 34,
   and
ii. comprises one or more amino acid substitutions in the HEXXH catalytic domain, where the HEXXH catalytic domain corresponds to amino acid positions 352-356 of SEQ ID NO: 34,
   wherein the polypeptide has reduced catalytic capacity and wherein the substitution(s) are preferably non-conservative.

These variants of Aureolysin have been demonstrated to induce antibodies that effectively target wild-type Aureolysin, but due to their reduced biological activity they are safe and pharmacologically acceptable.

In a first embodiment of the fourth aspect of the invention, in the variant, the conserved cysteine (C), corresponding to amino acid position 479 of SEQ ID NO: 34, is substituted.

In a second embodiment of the fourth aspect of the invention, the conserved cysteine, as defined in the first embodiment of the fourth aspect of the invention, is substituted with serine(S).

In a third embodiment of the fourth aspect of the invention, the glutamic acid residue (E) in the HEXXH catalytic domain, corresponding to amino acid position 353 of SEQ ID NO: 34, is substituted, preferably non-conservatively.

In a fourth embodiment of the fourth aspect of the invention, the glutamic acid in the HEXXH catalytic domain, as defined in the third embodiment of the fourth aspect of the invention, is substituted with alanine (A).

In a fifth embodiment of the fourth aspect of the invention, the variant consists of or comprises SEQ ID NO: 35.

In a 6^{th} embodiment of the fourth aspect of the invention, the variant comprises a sequence with at least 85% sequence identity with amino acid sequence 210-509 of SEQ ID NO: 34.

In a 7^{th} embodiment of the fourth aspect of the invention, the variant shows reduced ability to participate in forming antigen complexes as compared to wildtype Aur.

In an 8^{th} embodiment of the fourth aspect of the invention, the polypeptide can induce antibodies that block the catalytic activities of wildtype Aur.

### Embodiments of the 5^{th} Aspect of the Invention

The fifth aspect of the invention relates to an immunogenic polypeptide consisting of or comprising a variant of the amino acid sequence of N-acetylmuramoyl-L-alanine amidase (SAR2723), which variant:
i. has at least 85% sequence identity with the amino acid sequence of SEQ ID NO: 38, and
   a. comprises one or more amino acid substitutions in the amidase active site TXEXX domain corresponding to amino acid residues 384-388 in SEQ ID NO: 37, and/or
   b. comprises one or more substitutions in the amidase active site LXDYX domain corresponding to amino acid residues 409-413 in SEQ ID NO: 37, and/or
   c. comprises a substitution of a conserved cysteine corresponding to position 513 in SEQ ID NO: 37,
      wherein the polypeptide has reduced catalytic capacity and wherein the substitution(s) are preferably non-conservative.

These variants of SAR2723 have been demonstrated to induce antibodies that effectively target wild-type SAR2723 but due to their reduced biological activity they are—like the other polypeptides of the invention disclosed above-safe and pharmacologically acceptable.

In a first embodiment of the fifth aspect of the invention, the immunogenic polypeptide comprises the following of features i: a only, b only, c only, a and b only, a and c only, b and c only, or a, b and c.

In a second embodiment of the fifth aspect of the invention, the conserved cysteine is substituted with serine(S).

In a third embodiment of the fifth aspect of the invention, the glutamic acid residue (E) in the active site TXEXX domain, corresponding to amino acid position 386 of SEQ ID NO: 37, is substituted, preferably non-conservatively, such as with glutamine (Q).

In a fourth embodiment of the fifth aspect of the invention, the aspartic acid residue (D) in the active site LXDYS domain corresponding to amino acid position 411 of SEQ ID NO: 37, is substituted, preferably non-conservatively, such as with asparagine (N).

In a fifth embodiment of the fifth aspect of the invention, the variant consists of or comprises SEQ ID NO: 39.

In a 6^{th} embodiment of the fifth aspect of the invention, the polypeptide can induce antibodies that block the catalytic activities of wildtype SAR2723 or that interfere with bacterial cell wall development.

### Embodiments of the 6^{th} Aspect of the Invention

The 6^{th} aspect of the invention relates to a vaccine composition comprising a selection of polypeptides, which includes at least 2 of a-d:
a) an Hla polypeptide or a variant thereof, where said variant preferably exhibits reduced or no hemolytic activity and/or which preferably can induce antibodies that block the hemolytic activity of native Hla,
b) a LukE polypeptide or a variant thereof, where said variant preferably exhibits reduced or no leukocytic activity and/or which preferably can induce antibodies that block the leukocytic activity of native LukE,
c) an SpA polypeptide or a variant thereof, where said variant preferably exhibits a reduced ability to bind human IgG and human von Willebrandt Factor and/or which preferably can induce antibodies that block native SpA interaction with human IgG, and
d) an Aur polypeptide or a variant, thereof where said variant preferably exhibits reduced catalytic activity and which preferably can induce antibodies that block the catalytic activity of native Aur, said composition optionally further comprising a pharmaceutically acceptable carrier, vehicle or diluent, and further optionally an immunogenic adjuvant, which is in particular selected from AIOH, SLA-SE and OMVs (outer membrane vesicles).

It will be understood that such a composition can be composed of the above-discussed variants of SpA, Hla, LukE, and Aur, but that polypeptides known from the art that exhibit comparable functional properties also find use in the composition. However, it is in a first embodiment of the 6^{th} aspect of the invention preferred that the Hla polypeptide variant is according to the second aspect of the invention, and/or wherein the LukE polypeptide variant is according to the third aspect of the invention and/or wherein the SpA polypeptide variant is according to the first aspect of the present invention and/or wherein the Aur polypeptide variant is according to the fourth aspect of the invention.

In a second embodiment of the 6^{th} aspect of the invention, the Hla polypeptide variant has the amino acid sequence SEQ ID NO: 5 or any other previously identified Hla polypeptide, which shares the feature of not being capable of forming a heptameric (pore forming) structure. Likewise, this embodiment also utilizes any existing SpA polypeptide variant and/or Aur polypeptide variant(s), which share the functionality discussed above for the SpA and Aur polypeptide variants of the invention. This embodiment hence utilizes at least one immunogen which has the same or comparable properties exhibited by the polypeptides of 1^{st}-4^{th} aspect of the present invention.

In a third embodiment of the 6^{th} aspect of the invention, the vaccine composition comprises at least 3 of a-d.

In a fourth embodiment of the 6^{th} aspect of the invention, the vaccine composition comprises
a and b;
a and c;
a and d;
b and c;
b and d;
c and d;
a, b, and c;
a, b, and d;
a, c, and d;
b, c, and d; or
a, b, c, and d.

In particular, the vaccine composition, where the S. aureus immunogens comprise or consist of a and b are preferred, i.e. the vaccine composition, where the S. aureus immunogens comprise or consist of a) an Hla polypeptide or a variant thereof, where said variant preferably exhibits reduced or no hemolytic activity and/or which preferably can induce antibodies that block the hemolytic activity of native Hla, and
b) a LukE polypeptide or a variant thereof, where said variant preferably exhibits reduced or no leukocytic activity and/or which preferably can induce antibodies that block the leukocytic activity of native LukE, where each of a and b are as disclosed herein.

In a fifth embodiment of the 6^{th} aspect of the invention, the vaccine composition further comprises at least one polypeptide selected from
e) a SAR2723 polypeptide or a variant thereof, where said variant preferably exhibits reduced or no catalytic activity, and which can preferably induce antibodies that block the catalytic activity of native SAR2723,
f) a SAR2753 polypeptide or a variant, where said variant preferably exhibits reduced or no lipase activity and which can preferably induce antibodies that block the lipase activity of native SAR2753,
g) a SAR0992 (HtrA2) polypeptide or a variant thereof, and
h) a SAR0280 (EsaA) polypeptide of a variant thereof, where said variant can preferably induce antibodies that prevent EsxA/B secretion.

In a 6^{th} embodiment of the 6^{th} aspect of the invention,
the SAR0992 polypeptide variant is a fragment or sequence variant of SEQ ID NO: 15 as disclosed in WO 2012/136653, in particular a fragment consisting of amino acid residues 1-409 of SEQ ID NO: 15 in WO 2012/136653, or a variant of SAR0992 that can induce antibodies that block catalytic activity of native SAR0992, such as a truncate of SAR0992 C-terminal relative to the transmembrane helix of SAR0992, or is a mutated version of SAR0992 comprising a substitution of the serine residue corresponding to the serine residue in position 619 in SEQ ID NO: 15 disclosed in WO 2012/136653, and/or is a mutated version of SAR0992, where the transmembrane helix is exchanged with a flexible linker or wherein the transmembrane helix is exchanged with a linker and the N- and C-terminal parts of SAR0992 flanking the linker are exchanged with each other, and/or
wherein the SAR0280 polypeptide variant is a fragment or sequence variant of SEQ ID NO: 13 as disclosed in WO 2012/136653 or is a fusion of the most N-terminal and the most C-terminal extracellular fragments of SAR0280, and/or wherein
the SAR2723 polypeptide variant is according to the fifth aspect of the invention or a fragment or sequence variant of SEQ ID NO: 13 as disclosed in WO 2015/082536, and/or wherein
the SAR2753 polypeptide variant is a fragment or sequence variant of SEQ ID NO: 14 as disclosed in WO 2015/082536 or a polypeptide comprised of the amino acid sequence SEQ ID NO: 42.

In a 7^{th} embodiment of the 6^{th} aspect of the invention, the vaccine composition comprises at least 2 of e-h.

In an 8^{th} embodiment of the 6^{th} aspect of the invention, the vaccine composition comprises at least 3 of e-h.

In a 9^{th} embodiment of the 6^{th} aspect of the invention, the vaccine composition comprises
e and f;
e and g;
e and h;
f and g;
f and h;
g and h;
e, f, and g;
e, f, and h;
e, g, and h;
f, g, and h; or
e, f, g, and h.

More details relating to vaccine compositions is found below under the headings “immunization methods” and “compositions of the invention; vaccines”.

Also part of the 6^{th} aspect of the invention is a vaccine composition comprising at least one chimeric polypeptide of the 7^{th} aspect of the invention and detailed below under discussion of embodiments of the 7^{th} aspect of the invention, said vaccine composition further comprising a pharmaceutically acceptable carrier, vehicle or diluent, and further optionally an immunological adjuvant.

The at least one chimeric polypeptide in this embodiment is preferably selected from CHIM_LukE_mHla_H35L_FS (SEQ ID NO: 56), CHIM_mc2716_mSpA_7_12_FS (SEQ ID NO: 57), and CHIM_2753_mc27723_FS (SEQ ID NO: 59), and in particular from SEQ ID NO: 56 and 57.

In an important embodiment this vaccine composition comprises at least or exactly 2 chimeric polypeptides of the 7^{th} aspect of the invention and embodiments thereof.

In some embodiments, the vaccine composition, which comprises at least one chimeric polypeptide of the 7^{th} aspect, also comprises at least or exactly one further polypeptide selected from the polypeptides discussed above as e, g, f, and h in the embodiments of the 6^{th} aspect of the invention; i.e. such a composition comprises at least one chimeric polypeptide and at least one of the polypeptides defined as e, g, f, or h above.

The further polypeptide is preferably selected from the group consisting of SAR0992-1-409 (residues 1-409 of SEQ ID NO: 44) and SAR0280-28-820 (SEQ ID NO: 48).

A particularly important vaccine composition comprises chimeric polypeptides CHIM_LukE_mHla_H35L_FS (SEQ ID NO: 56) and CHIM_mc2716_mSpA_7_12_FS (SEQ ID NO: 57), and optionally the polypeptide consisting of amino acid residues 1-409 of SAR0992 (residues 1-409 of SEQ ID NO: 44), and a particularly important composition consists of chimeric polypeptides CHIM_LukE_mHla_H35L_FS (SEQ ID NO: 56) and

CHIM_mc2716_mSpA_7_12_FS (SEQ ID NO: 57), and optionally the polypeptide consisting of amino acid residues 1-409 of SAR0992 (residues 1-409 of SEQ ID NO: 44) in admixture with a pharmaceutically acceptable carrier, vehicle or diluent, and further optionally an immunological adjuvant.

The adjuvant is in preferred embodiments selected from the group consisting of AIOH, SLA-SE and OMVs (outer membrane vesicles).

### Embodiments of the 7^{th} Aspect of the Invention

The 7^{th} aspect of the invention relates to a chimeric polypeptide comprising amino acid sequences of the selection of polypeptides according to the 6^{th} aspect of the invention, wherein the amino acid sequences are fused or connected via a linker. In other words, instead of including some or all of the polypeptides discussed in the 6^{th} aspect of the invention into a vaccine “cocktail”, the immunogenic amino acid sequences are instead part of fusion proteins; this approach may facilitate production of the vaccine or can obviate any possible need for inclusion of immunogenic carrier molecules fused to the polypeptides.

Particularly preferred chimeric polypeptides are composed of or comprises the polypeptides a and b discussed above under the 4^{th} embodiment of the 6^{th} aspect above. In particular, the chimeric polypeptide is composed of or comprises the S. aureus immunogens a) an Hla polypeptide or a variant thereof, where said variant preferably exhibits reduced or no hemolytic activity and/or which preferably can induce antibodies that block the hemolytic activity of native Hla, and
b) a LukE polypeptide or a variant thereof, where said variant preferably exhibits reduced or no leukocytic activity and/or which preferably can induce antibodies that block the leukocytic activity of native LukE, where each of a and b are as disclosed herein.

In a first embodiment of the 7^{th} aspect of the invention, the linker is flexible or rigid.

In a second embodiment of the 7^{th} aspect of the invention, the flexible linker, as presented in the first embodiment of the 7^{th} aspect of the invention, is GSGGGA (SEQ ID NO: 50) or GSGGGAGSGGGA (SEQ ID NO: 51), or the rigid linker, as presented in the first embodiment of the 7^{th} aspect of the invention, is KPEPKPAPAPKP (SEQ ID NO: 52).

Particularly preferred chimeric polypeptides are composed of or comprises the polypeptides a and b discussed above under the 4^{th} embodiment of the 6^{th} aspect above. In particular, the chimeric polypeptide is composed of or comprises the S. aureus immunogens a) an Hla polypeptide or a variant thereof disclosed herein, where said variant preferably exhibits reduced or no hemolytic activity and/or which preferably can induce antibodies that block the hemolytic activity of native Hla, and b) a LukE polypeptide or a variant thereof detailed herein, where said variant preferably exhibits reduced or no leukocytic activity and/or which preferably can induce antibodies that block the leukocytic activity of native LukE, where each of a and b are as disclosed herein.

In a third embodiment of the 7^{th} aspect of the invention, the chimeric polypeptide hence has an amino acid sequence selected from the group consisting of CHIM_LukE_mHla_H35L_FS (SEQ ID NO: 56), CHIM_mc2716_mSpA_7_12_FS (SEQ ID NO: 57), CHIM_0992_mSpA_7_12_FS (SEQ ID NO: 58), CHIM_2753_mc2723_FS (SEQ ID NO: 59), CHIM_0280_2753_FS (SEQ ID NO: 60), CHIM_0992_2753_FS (SEQ ID NO: 61), CHIM_mc2723_Hla_H35L_t_FS (SEQ ID NO: 62), CHIM_mc2716_LukE_FS (SEQ ID NO: 63), CHIM_LukE_Hla_H35L_t_FS (SEQ ID NO: 64), CHIM_mc2716_mSpA_10_12_FS (SEQ ID NO: 65), CHIM_LukE_0280_FS (SEQ ID NO: 66), CHIM_m0992_0992_FL (SEQ ID NO: 67), CHIM_0992_Hla_H35L_t_FS (SEQ ID NO: 68), CHIM_LukE_mSpA_10_12_FS (SEQ ID NO: 82), CHIM_mc2723_mSpA_10_12_FS (SEQ ID NO: 83), CHIM_0992_mSpA_10_12_FS (SEQ ID NO: 84), and CHIM_LukE_mSpA_7_12_FS (SEQ ID NO: 85).

### Embodiments of the 8^{th}-12^{th} Aspects of the Invention

The 8^{th} aspect of the invention relates to a nucleic acid or nucleic acid fragment, which encodes a polypeptide of the present invention, whereas the 9^{th} aspect of the invention relates to a vector comprising the nucleic acid fragment according to the 8^{th} aspect of the invention. Likewise, the 10^{th}-12^{th} aspects relate to cells and composition that utilize vectors and nucleic acids disclosed herein; a more detailed discussion these embodiments are found infra.

### Embodiments of the 13^{th} Aspect of the Invention

The 13^{th} aspect of the invention relates to a method for inducing immunity in an animal by administering at least once an immunogenically effective amount of the immunogenic polypeptide according to any one of the first to fifth aspects of the invention, a vaccine composition according to the 6^{th} aspect of the invention, a chimeric polypeptide according to the 7^{th} aspect of the invention, a nucleic acid fragment according to the 8^{th} aspect of the invention, a vector according to the 9^{th} aspect of the invention, a transformed cell or virus according to the 10^{th} aspect of the invention, or an immunogenic composition according to the 11^{th} or 12^{th} aspect of the invention, so as to induce adaptive immunity against S. aureus in the animal.

In a first embodiment of the 13^{th} aspect of the invention, when the immunogenic polypeptide according to any one of the first to fifth aspects of the invention, the chimeric polypeptide according to the 7^{th} aspect of the invention, or a composition comprising said immunogenic polypeptide or said chimeric polypeptide is administered, the animal receives between 0.5 and 5,000 μg of the immunogenic polypeptide or the chimeric polypeptide according to any one of the first to fifth aspects and the 7^{th} aspect of the invention per administration.

In a second embodiment of the 13^{th} aspect of the invention, the animal receives a first priming administration comprising of said immunogenic polypeptide or said chimeric polypeptide and one or more booster administrations comprising said immunogenic polypeptide or said chimeric polypeptide.

In a third embodiment of the 13^{th} aspect of the invention, the animal is a human being.

In a fourth embodiment of the 13^{th} aspect of the invention, the administration is for the purpose of inducing protective immunity against S. aureus.

In a fifth embodiment of the 13^{th} aspect of the invention, the protective immunity is effective in reducing the risk of attracting infection with S. aureus or is effective in treating or ameliorating infection with S. aureus.

In a 6^{th} embodiment of the 13^{th} aspect of the invention, the administration is for the purpose of inducing antibodies specific for S. aureus and wherein said antibodies or B-lymphocytes producing said antibodies are subsequently recovered from the animal.

In a 7^{th} embodiment of the 13^{th} aspect of the invention, the administration is for the purpose of inducing antibodies specific for S. aureus and wherein B-lymphocytes producing said antibodies are subsequently recovered from the animal and used for preparation of monoclonal antibodies.

More details pertaining to this aspect is found infra in the sections detailing immunization methods and vaccines.

### Vectors

It will be understood that the nucleic acid fragments of the invention and nucleic acid fragment encoding polypeptides of the compositions of the invention may be used for both production, carrier and vaccine purposes—the latter will require that the sequences are included in expression vectors that may lead to production of immunogenic proteins in the mammal receiving the vector. Or put differently, the nucleic acid is comprised in a vector capable of expressing the nucleic acid in man upon administration.

Such a vector often comprises in operable linkage and in the 5′-3′ direction, an expression control region comprising an enhancer/promoter for driving expression of the nucleic acid, an optional signal peptide coding sequence, a nucleotide sequence to be expressed, and optionally a terminator. Hence, such a vector constitutes an expression vector useful for effecting production in cells of the polypeptide of the invention or a polypeptide being part of a composition of the invention. Since the polypeptides are bacterial of origin, recombinant production has to be effected in host cells that can express the coding nucleic acid. Bacterial host cells may preferably be used. However, if the vector is to drive expression in eukaryotic cell (as would be the case for a nucleic acid vaccine vector), the expression control region should be adapted to this particular use.

For production purposes it is therefore often convenient that the expression control region drives expression in a prokaryotic cell such as a bacterium, e.g. in E. coli, or in a eukaryotic cell such as a plant cell, an insect cell, or a mammalian cell. For vaccine purposes, the expression control region has to be able to drive expression in a mammalian, preferably human, cell.

Also, for production purposes, it is practical that the vector is capable of integrating the nucleic acid into the genome of a host cell—this is particularly useful if the vector is use in the production of stably transformed cells, where the progeny will also include the genetic information introduced via the vector. Alternatively, vectors incapable of being integrated into the genome of a piscine host cell are useful in e.g. nucleic acid vaccination.

An interesting production system is the use of plants. For instance, proteins can be produced at low cost in plants using an Agrobacterium transfection system to genetically modify plants to express genes that encode the protein of interest. One commercially available platform are those provided by iBio CMO LLC (8800 HSC Pkwy, Bryan, TX 77807, USA) and iBio, Inc (9 Innovation Way, Suite 100, Newark, DE 19711, USA) and disclosed in e.g. EP 2 853 599, EP 1 769 068, and EP 2 192 172. Hence, in such systems the vector is an Agrobacterium vector or other vector suitable for transfection of plants.

The vector is typically selected from the group consisting of a virus, such as a virus which is non-pathogenic in mammals and in particular in humans, a bacterium such as a bacterium which is non-pathogenic in mammals such as humans, a plasmid, a minichromosome, and a cosmid.

Interesting vectors are viral vectors (in particular those useful as vaccine agents in humans). These may be selected from the group consisting of a retrovirus vector, such as a lentivirus vector, an adenovirus vector, an adeno-associated virus vector, and a pox virus vector. Certain pox virus vectors are preferred, in particular vaccinia virus vectors. A particularly preferred vaccinia virus vector is a modified vaccinia Ankara (MVA) vector.

Polypeptides of the invention or being part of a composition of the invention may as indicated be encoded by a nucleic acid molecule comprised in a vector. A nucleic acid sequence can be “heterologous,” which means that it is in a context foreign to the cell in which the vector is being introduced, which includes a sequence homologous to a sequence in the cell but in a position within the host cell where it is ordinarily not found.

Vectors include naked DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques. In addition to encoding the polypeptides of this invention of being part of a composition of this invention, a vector may encode polypeptide sequences such as a “tag” or immunogenicity enhancing peptide (e.g. an immunogenic carrier or a fusion partner that stimulates the immune system, such as a cytokine or active fragment thereof). Useful vectors encoding such fusion proteins include pIN vectors, vectors encoding a stretch of histidine residues, and pGEX vectors, for use in generating glutathione S-transferase (GST) soluble fusion proteins for later purification and separation or cleavage.

Vectors may be used in a host cell to produce a polypeptide of the invention or a polypeptide being part of a composition of the inventio that may subsequently be purified for administration, or the vector may be purified for direct administration for expression of the protein (as is the case when administering a nucleic acid vaccine).

Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

### 1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including polymerase chain reaction in connection with the compositions disclosed herein.

It may be important to employ a promoter and/or enhancer that effectively direct(s) the expression of the DNA segment in the cell type or organism chosen for expression. Those of skill in the art of molecular biology generally know the use of promoters, enhancers, and cell type combinations for protein expression. The promoters employed may be constitutive, tissue-specific, or inducible and in certain embodiments may direct high level expression of the introduced DNA segment under specified conditions, such as large-scale production of recombinant proteins or peptides.

Examples of inducible elements, which are regions of a nucleic acid sequence that can be activated in response to a specific stimulus, include but are not limited to Immunoglobulin Heavy Chain, Immunoglobulin Light Chain, T Cell Receptor, HLA DQα and/or DQβ, β-Interferon, Interleukin-2, Interleukin-2 Receptor, MHC Class II 5, MHC Class II HLA-DRα, β-Actin, Muscle Creatine Kinase (MCK), Prealbumin (Transthyretin), Elastase I, Metallothionein (MTII), Collagenase, Albumin, α-Fetoprotein, γ-Globin, β-Globin, c-fos, c-HA-ras, Insulin, Neural Cell Adhesion Molecule (NCAM), αI-Antitrypain, H2B (TH2B) Histone, Mouse and/or Type I Collagen, Glucose-Regulated Proteins (GRP94 and GRP78), Rat Growth Hormone, Human Serum Amyloid A (SAA), Troponin I (TN I), Platelet-Derived Growth Factor (PDGF), Duchenne Muscular Dystrophy, SV40, Polyoma, Retroviruses, Papilloma Virus, Hepatitis B Virus, Human Immunodeficiency Virus, Cytomegalovirus (CMV) IE, and Gibbon Ape Leukemia Virus.

Inducible Elements include MT II—Phorbol Ester (TFA)/Heavy metals; MMTV (mouse mammary tumour virus)—Glucocorticoids; β-Interferon—poly(rl)x/poly(rc); Adenovirus 5 E2—EIA; Collagenase—Phorbol Ester (TPA); Stromelysin—Phorbol Ester (TPA); SV40—Phorbol Ester (TPA); Murine MX Gene—Interferon, Newcastle Disease Virus; GRP78 Gene—A23187; α-2-Macroglobulin—IL-6; Vimentin—Serum; MHC Class I Gene H-2κb—Interferon; HSP70—EIA/SV40 Large T Antigen; Proliferin—Phorbol Ester/TPA; Tumour Necrosis Factor—PMA; and Thyroid Stimulating Hormonea Gene—Thyroid Hormone.

Also contemplated as useful in the present invention are the dectin-1 and dectin-2 promoters. Additionally, any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

The particular promoter that is employed to control the expression of peptide or protein encoding polynucleotides is not believed to be critical, so long as it is capable of expressing the polynucleotide in a targeted cell. Where a piscine cell is targeted (as is the case in nucleic acid vaccination), it is preferable to position the polynucleotide coding region adjacent to and under the control of a promoter that is capable of being expressed in a piscine cell. Generally speaking, such a promoter might include either a bacterial, piscine or viral promoter as long as the promoter is effective in piscine cells.

In various embodiments—in particular those where recombinant production of the polypeptide is the aim—the human cytomegalovirus (CMV) immediate early gene promoter, the SV40 early promoter, and the Rous sarcoma virus long terminal repeat can be used to obtain high level expression of a related polynucleotide. The use of other viral or mammalian cellular or bacterial phage promoters, which are well known in the art, to achieve expression of polynucleotides is contemplated as well.

In embodiments in which a vector is administered to humans for expression of the protein, it is contemplated that a desirable promoter for use with the vector is one that is not down-regulated by cytokines or one that is strong enough that even if down-regulated, it produces an effective amount of the protein/polypeptide of the current invention (or useful in a composition of the present invention) in humans to elicit an immune response. Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells and macrophages. The mammalian MHC I and MHC II promoters are examples of such tissue-specific promoters in man and it is contemplated that corresponding piscine promoters will be effective.

### 2. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

### 3. Multiple Cloning Sites

Vectors can include a multiple cloning site (MCS), which is a nucleic acid region that contains multiple restriction enzyme sites, any of which can be used in conjunction with standard recombinant technology to digest the vector. Frequently, a vector is linearized or fragmented using a restriction enzyme that cuts within the MCS to enable exogenous sequences to be ligated to the vector. Techniques involving restriction enzymes and ligation reactions are well known to those of skill in the art of recombinant technology.

### 4. Splicing Sites

Most transcribed eukaryotic RNA molecules will undergo RNA splicing to remove introns from the primary transcripts. If relevant, vectors containing genomic eukaryotic sequences may require donor and/or acceptor splicing sites to ensure proper processing of the transcript for protein expression.

### 5. Termination Signals

The vectors or constructs disclosed herein will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the bovine growth hormone terminator or viral termination sequences, such as the SV40 terminator. In certain embodiments, the termination signal may be a lack of transcribable or translatable sequence, such as due to a sequence truncation.

### 6. Polyadenylation Signals

In expression, particularly eukaryotic expression (as is relevant in nucleic acid vaccination), one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

### 7. Origins of Replication

In order to propagate a vector in a host cell, it may contain one or more origins of replication sites (often termed “on”), which is a specific nucleic acid sequence at which replication is initiated. Alternatively, an autonomously replicating sequence (ARS) can be employed if the host cell is yeast.

### 8. Selectable and Screenable Markers

In certain embodiments of the invention, cells containing a nucleic acid construct may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein of the invention. Further examples of selectable and screenable markers are well known to one of skill in the art.

### Transformed Cells

Transformed cells are useful as organisms for producing the polypeptide of the invention the polypeptide of the composition of the invention, but also as simple “containers” of nucleic acids and vectors of the invention.

Certain transformed cells, including those of the invention, are capable of replicating a nucleic acid fragment, including the nucleic acid fragment of the invention. Preferred transformed cells are capable of expressing the nucleic acid fragment.

For recombinant production it is, since the proteins disclosed herein are of bacterial origin, convenient that the transformed cell is prokaryotic, such as a bacterium, but generally both prokaryotic cells and eukaryotic cells may be used.

Suitable prokaryotic cells are bacterial cells (preferably non-pathogenic) selected from the group consisting of Escherichia (such as E. coli.), Bacillus (e.g. Bacillus subtilis), Salmonella, and Mycobacterium (e.g. M. bovis BCG).

Eukaryotic cells can be in the form of yeasts (such as Saccharomyces cerevisiae) and protozoans. Alternatively, the transformed eukaryotic cells are derived from a multicellular organism such as a filamentous fungus, an insect cell, a plant cell, or a mammalian cell.

For production purposes, it is advantageous that the transformed cell is stably transformed by having the nucleic acid stably integrated into its genome, and in certain embodiments it is also preferred that the transformed cell secretes or carries on its surface the polypeptide of the invention, since this facilitates recovery of the polypeptides produced. A particular version of this embodiment is one where the transformed cell is a bacterium and secretion of the polypeptide of the invention is into the periplasmic space.

As noted above, stably transformed cells are preferred—these i.a. allows that cell lines comprised of transformed cells as defined herein may be established—such cell lines are particularly preferred.

Further details on cells and cell lines are presented in the following:

Suitable cells for recombinant nucleic acid expression of the nucleic acid fragments, such as those of the present invention, are prokaryotes and eukaryotes. Examples of prokaryotic cells include E. coli; members of the Staphylococcus genus, such as S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the Pseudomonas genus such as Ps. fluorescens. Examples of eukaryotic cells include mammalian cells; insect cells; yeast cells such as members of the Saccharomyces genus (e.g. S. cerevisiae), members of the Pichia genus (e.g. P. pastoris), members of the Hansenula genus (e.g. H. polymorpha), members of the Kluyveromyces genus (e.g. K. lactis or K. fragilis) and members of the Schizosaccharomyces genus (e.g. S. pombe).

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2 nd Edition, Cold Spring Harbor Laboratory Press, 1989.

As used herein, the terms “cell,” “cell line,” and “cell culture” may be used interchangeably. All of these terms also include their progeny, which includes any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org) or from other depository institutions such as Deutsche Sammlung vor Microorganismen und Zellkulturen (DSM). An appropriate host can be determined by one of skill in the art based on the vector backbone and the desired result. A plasmid or cosmid, for example, can be introduced into a prokaryote host cell for replication of many vectors or expression of encoded proteins. Bacterial cells used as host cells for vector replication and/or expression include Staphylococcus strains, DH5a, JMI 09, and KC8, as well as a number of commercially available bacterial hosts such as SURE® Competent Cells and SOLOP ACK™ Gold Cells (STRATAGENER, La Jolla, CA). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ Baculovirus expression system from CLONTECH®

In addition to the disclosed expression systems, other examples of expression systems include STRATAGENER's COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter.

INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### Methods of Gene Transfer

Suitable methods for nucleic acid delivery to effect expression of compositions or nucleic acid fragments of the present invention are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859), including microinjection (U.S. Pat. No. 5,789,215); by electroporation (U.S. Pat. No. 5,384,253); by calcium phosphate precipitation; by using DEAE dextran followed by polyethylene glycol; by direct sonic loading; by liposome mediated transfection; by microprojectile bombardment (PCT Application Nos. WO 94/09699 and 95/06128; U.S. Pat. Nos. 5,610,042; 5,322,783 5,563,055, 5,550,318, 5,538,877 and 5,538,880); by agitation with silicon carbide fibers (U.S. Pat. Nos. 5,302,523 and 5,464,765); by Agrobacterium mediated transformation (U.S. Pat. Nos. 5,591,616 and 5,563,055); or by PEG mediated transformation of protoplasts (U.S. Pat. Nos. 4,684,611 and 4,952,500); by desiccation/inhibition mediated DNA uptake. Through the application of techniques such as these, organelle(s), cell(s), tissue(s) or organism(s) may be stably or transiently transformed.

### Compositions of the Invention; Vaccines

Compositions, in particular vaccines, according to the invention are prophylactic but may also be used therapeutically.

Such vaccines comprise immunising antigen(s), immunogen(s), polypeptide(s), protein(s) or nucleic acid(s), usually in combination with “pharmaceutically acceptable carriers”, which include any carrier that does not itself induce the production of antibodies harmful to the individual receiving the composition.

In some embodiments of the invention, the pharmaceutical compositions such as vaccines include merely one single antigen, immunogen, polypeptide, protein, nucleic acid or vector of the invention, but in other embodiments, the pharmaceutical compositions comprise “cocktails” of antigens or immunogens or polypeptides or protein or nucleic acids or vectors.

In interesting embodiments, the pharmaceutical composition is a vector mentioned herein, which encodes and can effect expression of at least 2 nucleic acid fragments of the invention.

Another interesting embodiment of a pharmaceutical composition comprises RNA as the active principle, i.e. at least one mRNA encoding a polypeptide of the invention.

An embodiment of a pharmaceutical composition of the invention comprises at least 2 (such as 2, 3, 4, 5, 6, 7, 8, 9, or 10) distinct polypeptides as described above.

Another embodiment of the pharmaceutical composition of the invention comprises at least 2 (such as 2, 3, 4, 5, 6, 7, 8, 9, or 10) distinct nucleic acid molecules (such as DNA and RNA) each encoding a polypeptide discussed above.

Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art. Additionally, these carriers may function as immune stimulating agents (“adjuvants”). Furthermore, the antigen or immunogen may be conjugated to a bacterial toxoid, such as a toxoid from diphtheria, tetanus, cholera, H. pylori, etc.

The pharmaceutical compositions of the invention thus typically contain an immunological adjuvant, which is commonly an aluminium based adjuvant or one of the other adjuvants described in the following:

Preferred adjuvants to enhance effectiveness of the composition include, but are not limited to: (1) aluminium salts (alum), such as aluminium hydroxide (AIOH), aluminium phosphate, aluminium sulphate, etc; (2) oil-in-water emulsion formulations (with or without other specific immune stimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, MA), (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) Ribi adjuvant system (RAS), (Ribi Immunochem, Hamilton, MT) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™); (3) saponin adjuvants such as Stimulon™ (Cambridge Bioscience, Worcester, MA) may be used or particles generated therefrom such as ISCOMs (immune stimulating complexes); (4) Complete Freund's Adjuvant (CFA) and Incomplete Freund's Adjuvant (IFA); (5) cytokines, such as interleukins (eg. IL-1, IL-2, IL-4, IL-5, IL-6, IL-7, IL-12, etc.), interferons (e.g. gamma interferon), macrophage colony stimulating factor (M-CSF), tumour necrosis factor (TNF), etc.; and (6) other substances that act as immune stimulating agents to enhance the effectiveness of the composition. Alum and MF59™ adjuvants are preferred together with CFA and IFA.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

Other interesting adjuvants are disclosed in Didierlaurent A M et al., Expert Rev. Vaccines 2017; 16: 55-63, Gonzales-Lopez A. et al., Clin. Immunol. 2019; 209, 108275. Further, the TLR4 ligand adjuvants, such as SLA-SE, are also preferred adjuvant systems (cf. Reed S. G. et al., Current Opinion in Immunology 2016, 41: 85-90; Liang H. et al., npj Vaccines 2020, 4:19, doi.org/10.1038/s41541-019-0116-6; van Hoeven N. et al., PLOS One, 2016, DOI:10.1371/journal.pone.0149610; and Reed S. G. et al., Seminars in Immunology 2018, 39: 22-29).

Another preferred adjuvant system is disclosed in Agger E M et al., PLoS One. 2008; 3(9): e3116; van Dissel J D et al., Vaccine. 2014 Dec. 12; 32(52):7098-107. doi: 10.1016/j.vaccine.2014.10.036. Epub 2014 Oct. 30; and Dietrich J et al., PLoS One. 2014 Jun. 23; 9(6):e100879. doi: 10.1371/journal.pone.0100879. eCollection 2014.

Another particularly preferred adjuvant system is OMVs (outer membrane vesicles), cf. Kaparakis-Liaskos, M. & Ferrero, R. L., 2015. Immune modulation by bacterial outer membrane vesicles. Nat Rev Immunol. 15:375-387; Russo, A. J. et al., 2018. Emerging insights into noncanonical inflammasome recognition of microbes. J Mol Biol. 430:207-216; and Tan K et al., Front Microbiol. 2018; 9: 783; doi: 10.3389/fmicb.2018.00783. In one interesting embodiment, OMVs are used in combination with Aluminium hydroxide.

Another possibility for a polypeptide vaccine formulation is to include the vaccine polypeptide(s) in a virus-like particle, i.e. a non-infectious self-assembling structure composed of envelope or capsid proteins, where the protein(s) are incorporated. The effect is multiple presentations of the polypeptides of the invention on the surface of the VLP, which in turn provides for improved immune recognition of the polypeptides. Hence, VLPs exert immunological adjuvant effects, too.

The immunogenic compositions (e.g. the immunising antigen or immunogen or polypeptide or protein or nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic polypeptide compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of the individual to be treated (e.g. non-human primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount of immunogen will fall in a relatively broad range that can be determined through routine trials. However, for the purposes of protein vaccination, the amount administered per immunization is typically in the range between 0.5 μg and 500 mg (however, often not higher than 5,000 μg). The amount of polypeptide of the invention can therefore be between 1 and 400 μg, between 2 and 350 μg, between 4 and 300 μg, between 5 and 250 μg, and between 10 and 200 μg. Hence, the composition will typically contain between 0.1-500 μg of protein of the invention per g of vaccine composition.

The immunogenic compositions are conventionally administered parenterally, e.g., by injection, either subcutaneously, intramuscularly, or transdermally/transcutaneously (cf. e.g. W0 98/20734). Additional formulations suitable for other modes of administration include oral and pulmonary formulations, suppositories, and transdermal applications. In the case of nucleic acid vaccination, also the intravenous or intraarterial routes may be applicable.

Dosage treatment may be a single dose schedule or a multiple dose schedule. The vaccine may be administered in conjunction with other immunoregulatory agents.

As an alternative to protein-based vaccines, DNA vaccination (also termed nucleic acid vaccination or gene vaccination) may be used (cf. e.g. Robinson & Torres (1997) Seminars in Immunol 9: 271-283; Donnelly et al. (1997) Annu Rev Immunol 15: 617-648).

A further aspect of the invention is as mentioned above the recognition that combination vaccines can be provided, wherein 2 or more polypeptide antigens disclosed herein are combined to enhance the immune response by the vaccinated individual, including to optimize initial immune response and duration of immunity. For the purposes of this aspect of the invention, multiple antigenic fragments derived from the same, longer protein can also be used, such as the use of a combination of different lengths of polypeptide sequence fragments from one protein.

Thus, embodiments of the invention relate to a composition (or the use as a vaccine thereof) comprising 2 distinct (i.e. non-identical) proteinaceous immunogens disclosed herein.

### Immunization Methods

The method of this aspect of the invention generally relates to induction of immunity and as such also entails methods that are prophylactic as well as therapeutic.

When immunization methods entail that a polypeptide of the invention or a polypeptide composition of the invention is administered the animal (e.g. the human) typically receives between 0.5 and 5,000 μg of the polypeptide per administration, cf. the above indications concerning dosages.

In preferred embodiments, the immunization scheme includes that a primary administration of the chimeric polypeptide(s), the nucleic acids/vectors, or the composition(s) of the invention is made, but it may be necessary to follow up with one or more booster administrations.

Preferred embodiments comprise that the administration is for the purpose of inducing protective immunity against S. aureus. In this embodiment it is particularly preferred that the protective immunity is effective in reducing the risk of attracting infection with S. aureus.

Some vaccine compositions of the invention induce humoral immunity, so it is preferred that the administration is for the purpose of inducing antibodies specific for S. aureus. But, as also mentioned the immunization method may also be useful in antibody production, so in other embodiments the administration is for the purpose of inducing antibodies specific for S. aureus wherein B-lymphocytes producing said antibodies are subsequently recovered from the animal and used for preparation of monoclonal antibodies.

Compositions for immunization can as mentioned above comprise polypeptides, nucleic acids, vectors virus or cells. The pharmaceutical compositions will comprise a therapeutically effective amount thereof.

The term “therapeutically effective amount” or “prophylactically effective amount” as used herein refers to an amount of a therapeutic agent to treat, ameliorate, or prevent a desired disease or condition, or to exhibit a detectable preventative effect in a group of mammals such as humans The effect can be detected by, for example, chemical markers or antigen levels. Reference is made to the ranges for dosages of immunologically effective amounts of polypeptides, cf. above. However, the effective amount for a given situation can be determined by routine experimentation and is within the judgement of the clinician.

For purposes of the present invention, an effective dose of a nucleic acid vaccine (vector) will be from about 0.01 mg/kg to 50 mg/kg or 0.05 mg/kg to about 10 mg/kg of the DNA or RNA constructs in the animal to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N. J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

### Specific Amino Acid Sequences Disclosed Herein

In the present application, the following amino acid sequences are referred to by both sequence ID NOs and separate designations (provided in parentheses) used herein:

| (Hla - wild type full length) | |
|---|---|
| SEQ ID NO: 1 | |
| MKTRIVSSVT TTLLLGSILM NPVANAADSD INIKTGTTDI GSNTTVKTGD LVTYDKENGM | 60 |
| HKKVFYSFID DKNHNKKLLV IRTKGTIAGQ YRVYSEEGAN KSGLAWPSAF KVQLQLPDNE | 120 |
| VAQISDYYPR NSIDTKEYMS TLTYGENGNV TGDDTGKIGG LIGANVSIGH TLKYVQPDFK | 180 |
| TILESPTDKK VGWKVIFNNM VNQNWGPYDR DSWNPVYGNQ LFMKTRNGSM KAADNFLDPN | 240 |
| KASSLLSSGF SPDFATVITM DRKASKQQTN IDVIYERVRD DYQLHWTSTN WKGTNTKDKW | 300 |
| IDRSSERYKI DWEKEEMTN | 319 |
| (Hla_wt-27-319) | |
| SEQ ID NO: | |
| ADSDINIKTG TTDIGSNTTV KTGDLVTYDK ENGMHKKVFY SFIDDKNHNK KLLVIRTKGT | 60 |
| IAGQYRVYSE EGANKSGLAW PSAFKVQLQL PDNEVAQISD YYPRNSIDTK EYMSTLTYGF | 120 |
| NGNVTGDDTG KIGGLIGANV SIGHTLKYVQ PDFKTILESP TDKKVGWKVI FNNMVNQNWG | 180 |
| PYDRDSWNPV YGNQLFMKTR NGSMKAADNF LDPNKASSLL SSGFSPDFAT VITMDRKASK | 240 |
| QQTNIDVIYE RVRDDYQLHW TSTNWKGTNT KDKWIDRSSE RYKIDWEKEE MTN | 293 |
| (Hla_t-39-319) | |
| SEQ ID NO: 3 | |
| DIGSNTTVKT GDLVTYDKEN GMHKKVFYSF IDDKNHNKKL LVIRTKGTIA GQYRVYSEEG | 60 |
| ANKSGLAWPS AFKVQLQLPD NEVAQISDYY PRNSIDTKEY MSTLTYGENG NVTGDDTGKI | 120 |
| GGLIGANVSI GHTLKYVQPD FKTILESPTD KKVGWKVIEN NMVNQNWGPY DRDSWNPVYG | 180 |
| NQLFMKTRNG SMKAADNFLD PNKASSLLSS GFSPDFATVI TMDRKASKQQ TNIDVIYERV | 240 |
| RDDYQLHWTS TNWKGTNTKD KWIDRSSERY KIDWEKEEMT N | 281 |
| (mHla-27-319) | |
| SEQ ID NO: 4 | |
| SETEVSVRSA SSDIGSNTTV KTGDLVTYDK ENGMHKKVFY SFIDDKNHNK KLLVIRTKGT | 60 |
| IAGQYRVYSE EGANKSGLAW PSAFKVQLQL PDNEVAQISD YYPRNSIDTK EYMSTLTYGF | 120 |
| NGNVTGDDTG KIGGLIGANV SIGHTLKYVQ PDFKTILESP TDKKVGWKVI ENNMVNQNWG | 180 |
| PYDRDSWNPV YGNQLFMKTR NGSMKAADNF LDPNKASSLL SSGFSPDFAT VITMDRKASK | 240 |
| QQTNIDVIYE RVRDDYQLHW TSTNWKGTNT KDKWIDRSSE RYKIDWEKEE MTN | 293 |
| (Hla_H35L-27-319) | |
| SEQ ID NO: 5 | |
| ADSDINIKTG TTDIGSNTTV KTGDLVTYDK ENGMLKKVFY SFIDDKNHNK KLLVIRTKGT | 60 |
| IAGQYRVYSE EGANKSGLAW PSAFKVQLQL PDNEVAQISD YYPRNSIDTK EYMSTLTYGF | 120 |
| NGNVTGDDTG KIGGLIGANV SIGHTLKYVQ PDFKTILESP TDKKVGWKVI FNNMVNQNWG | 180 |
| PYDRDSWNPV YGNQLFMKTR NGSMKAADNF LDPNKASSLL SSGFSPDFAT VITMDRKASK | 240 |
| QQTNIDVIYE RVRDDYQLHW TSTNWKGTNT KDKWIDRSSE RYKIDWEKEE MTN | 293 |
| (Hla_H35L_t-39-319) | |
| SEQ ID NO: 6 | |
| DIGSNTTVKT GDLVTYDKEN GMLKKVFYSF IDDKNHNKKL LVIRTKGTIA GQYRVYSEEG | 60 |
| ANKSGLAWPS AFKVQLQLPD NEVAQISDYY PRNSIDTKEY MSTLTYGENG NVTGDDTGKI | 120 |
| GGLIGANVSI GHTLKYVQPD FKTILESPTD KKVGWKVIEN NMVNQNWGPY DRDSWNPVYG | 180 |
| NQLFMKTRNG SMKAADNFLD PNKASSLLSS GFSPDFATVI TMDRKASKQQ TNIDVIYERV | 240 |
| RDDYQLHWTS TNWKGTNTKD KWIDRSSERY KIDWEKEEMT N | 281 |
| (mHla H35L-27-319) | |
| SEQ ID NO: 7 | |
| SETEVSVRSA SSDIGSNTTV KTGDLVTYDK ENGMLKKVFY SFIDDKNHNK KLLVIRTKGT | 60 |
| IAGQYRVYSE EGANKSGLAW PSAFKVQLQL PDNEVAQISD YYPRNSIDTK EYMSTLTYGF | 120 |
| NGNVTGDDTG KIGGLIGANV SIGHTLKYVQ PDFKTILESP TDKKVGWKVI ENNMVNQNWG | 180 |
| PYDRDSWNPV YGNQLFMKTR NGSMKAADNF LDPNKASSLL SSGFSPDFAT VITMDRKASK | 240 |
| QQTNIDVIYE RVRDDYQLHW TSTNWKGTNT KDKWIDRSSE RYKIDWEKEE MTN | 293 |
| (SpA_wild type full length) | |
| SEQ ID NO: 15 | |
| MMTLQIHTGG INLKKKNIYS IRKLGVGIAS VTLGTLLISG GVTPAANAAQ HDEAQQNAFY | 60 |
| QVLNMPNLNA DQRNGFIQSL KDDPSQSANV LGEAQKLNDS QAPKADAQQN NENKDQQSAF | 120 |
| YEILNMPNLN EAQRNGFIQS LKDDPSQSTN VLGEAKKLNE SQAPKADNNF NKEQQNAFYE | 180 |
| ILNMPNLNEE QRNGFIQSLK DDPSQSANLL SEAKKLNESQ APKADNKENK EQQNAFYEIL | 240 |
| HLPNLNEEQR NGFIQSLKDD PSQSANLLAE AKKLNDAQAP KADNKENKEQ QNAFYEILHL | 300 |
| PNLTEEQRNG FIQSLKDDPS VSKEILAEAK KLNDAQAPKE EDNNKPGKED NNKPGKEDNN | 360 |
| KPGKEDNNKP GKEDGNKPGK EDNKKPGKED GNKPGKEDNK KPGKEDGNKP GKEDGNKPGK | 420 |
| EDGNGVHVVK PGDTVNDIAK ANGTTADKIA ADNKLADKNM IKPGQELVVD KKQPANHADA | 480 |
| NKAQALPETG EENPFIGTTV FGGLSLALGA ALLAGRRREL | 520 |
| (SpA_wt_t: sequence listed in domain order E-D-A-B-C) | |
| SEQ ID NO: 16 | |
| AQHDEAQQNA FYQVLNMPNL NADQRNGFIQ SLKDDPSQSA NVLGEAQKLN DSQAPKADAQ | 60 |
| QNNENKDQQS AFYEILNMPN LNEAQRNGFI QSLKDDPSQS TNVLGEAKKL NESQAPKADN | 120 |
| NENKEQQNAF YEILNMPNLN EEQRNGFIQS LKDDPSQSAN LLSEAKKLNE SQAPKADNKF | 180 |
| NKEQQNAFYE ILHLPNLNEE QRNGFIQSLK DDPSQSANLL AEAKKLNDAQ APKADNKENK | 240 |
| EQQNAFYEIL HLPNLTEEQR NGFIQSLKDD PSVSKEILAE AKKLNDAQAP K | 291 |
| (SpA_10_12) | |
| SEQ ID NO: 17 | |
| AQVDEAQKNA FYQVLNMPNL NADQRNGFIQ SLKDAPSQSA KVLGEAQKLN DSQAPKADAQ | 60 |
| QNNVNKDQKS AFYEILNMPN LNEAQRNGFI QSLKDAPSQS TKVLGEAKKL NESQAPKADN | 120 |
| NVNKEQKNAF YEILNMPNLN EEQRNGFIQS LKDAPSQSAK LLSEAKKLNE SQAPKADNKV | 180 |
| NKEQKNAFYE ILHLPNLNEE QRNGFIQSLK DAPSQSAKLL AEAKKLNDAQ APKADNKVNK | 240 |
| EQKNAFYEIL HLPNLTEEQR NGFIQSLKDA PSVSKKILAE AKKLNDAQAP K | 291 |
| (SpA_7_12) | |
| SEQ ID NO: 18 | |
| AQVDEAQKNA FYQVLNMPNL NADQRNGFIQ SLKDAPAKSA NVLGEAQKLN DSQAPKADAQ | 60 |
| QNNVNKDQKS AFYEILNMPN LNEAQRNGFI QSLKDAPAKS TNVLGEAKKL NESQAPKADN | 120 |
| NVNKEQKNAF YEILNMPNLN EEQRNGFIQS LKDAPAKSAN LLSEAKKLNE SQAPKADNKV | 180 |
| NKEQKNAFYE ILHLPNLNEE QRNGFIQSLK DAPAKSANLL AEAKKLNDAQ APKADNKVNK | 240 |
| EQKNAFYEIL HLPNLTEEQR NGFIQSLKDA PAKSKEILAE AKKLNDAQAP K | 291 |
| (SpA_4_12) | |
| SEQ ID NO: 19 | |
| AQVDEAQKNA FYQVLNMPNL NADQRNGFIQ SLKRDPRQSA NVLGEAQKLN DSQAPKADAQ | 60 |
| QNNVNKDQKS AFYEILNMPN LNEAQRNGFI QSLKRDPRQS TNVLGEAKKL NESQAPKADN | 120 |
| NVNKEQKNAF YEILNMPNLN EEQRNGFIQS LKRDPRQSAN LLSEAKKLNE SQAPKADNKV | 180 |
| NKEQKNAFYE ILHLPNLNEE QRNGFIQSLK RDPRQSANLL AEAKKLNDAQ APKADNKVNK | 240 |
| EQKNAFYEIL HLPNLTEEQR NGFIQSLKRD PRVSKEILAE AKKLNDAQAP K | 291 |
| (SpA_3_12) | |
| SEQ ID NO: 20 | |
| AQVDEAQKNA FYQVLNMPNL NADQRNGFIQ SLKDRPRQSA NVLGEAQKLN DSQAPKADAQ | 60 |
| QNNVNKDQKS AFYEILNMPN LNEAQRNGFI QSLKDRPRQS TNVLGEAKKL NESQAPKADN | 120 |
| NVNKEQKNAF YEILNMPNLN EEQRNGFIQS LKDRPRQSAN LLSEAKKLNE SQAPKADNKV | 180 |
| NKEQKNAFYE ILHLPNLNEE QRNGFIQSLK DRPRQSANLL AEAKKLNDAQ APKADNKVNK | 240 |
| EQKNAFYEIL HLPNLTEEQR NGFIQSLKDR PRVSKEILAE AKKLNDAQAP K | 291 |
| (SpA_10_15) | |
| SEQ ID NO: 21 | |
| AQHDEAQKKA FYQVLNMPNL NADQRNGFIQ SLKDAPSQSA KVLGEAQKLN DSQAPKADAQ | 60 |
| QNNFNKDQKK AFYEILNMPN LNEAQRNGFI QSLKDAPSQS TKVLGEAKKL NESQAPKADN | 120 |
| NFNKEQKKAF YEILNMPNLN EEQRNGFIQS LKDAPSQSAK LLSEAKKLNE SQAPKADNKF | 180 |
| NKEQKKAFYE ILHLPNLNEE QRNGFIQSLK DAPSQSAKLL AEAKKLNDAQ APKADNKENK | 240 |
| EQKKAFYEIL HLPNLTEEQR NGFIQSLKDA PSVSKKILAE AKKLNDAQAP K | 291 |
| (wt_LukE) | |
| SEQ ID NO: 29 | |
| MFKKKMLAAT LSVGLIAPLA SPIQESRANT NIENIGDGAE VIKRTEDVSS KKWGVTQNVQ | 60 |
| FDFVKDKKYN KDALIVKMQG FINSRTSFSD VKGSGYELTK RMIWPFQYNI GLTTKDPNVS | 120 |
| LINYLPKNKI ETTDVGQTLG YNIGGNFQSA PSIGGNGSEN YSKTISYTQK SYVSEVDKQN | 180 |
| SKSVKWGVKA NEFVTPDGKK SAHDRYLFVQ SPNGPTGSAR EYFAPDNQLP PLVQSGENPS | 240 |
| FITTLSHEKG SSDTSEFEIS YGRNLDITYA TLFPRTGIYA ERKHNAFVNR NFVVRYEVNW | 300 |
| KTHEIKVKGH N | 311 |
| (LukE-29-311) | |
| SEQ ID NO: 30 | |
| NTNIENIGDG AEVIKRTEDV SSKKWGVTQN VQFDFVKDKK YNKDALIVKM QGFINSRTSF | 60 |
| SDVKGSGYEL TKRMIWPFQY NIGLTTKDPN VSLINYLPKN KIETTDVGQT LGYNIGGNFQ | 120 |
| SAPSIGGNGS FNYSKTISYT QKSYVSEVDK QNSKSVKWGV KANEFVTPDG KKSAHDRYLF | 180 |
| VQSPNGPTGS AREYFAPDNQ LPPLVQSGEN PSFITTLSHE KGSSDTSEFE ISYGRNLDIT | 240 |
| YATLFPRTGI YAERKHNAFV NRNFVVRYEV NWKTHEIKVK GHN | 283 |
| (USA300HOU_2637) | |
| SEQ ID NO: 33 | |
| MRKFSRYAFT SMAALTLLST LSPAALAIDS KNKPANSDIK FEVTQKSDAV KALKELPKSE | 60 |
| NVKNIYQDYA VTDVKTDKKG FTHYTLQPSV DGVHAPDKEV KVHADKSGKV VLINGDTDAK | 120 |
| KVKPTNKVTL SKDDAADKAF KAVKIDKNKA KNLKDKVIKE NKVEIDGDSN KYVYNVELIT | 180 |
| VTPEISHWKV KIDAQTGEIL EKMNLVKEAA ETGKGKGVLG DTKDININSI DGGFSLEDLT | 240 |
| HQGKLSAFSF NDQTGQATLI TNEDENFVKD EQRAGVDANY YAKQTYDYYK DTFGRESYDN | 300 |
| QGSPIVSLTH VNNYGGQDNR NNAAWIGDKM IYGDGDGRTF TSLSGANDVV AHELTHGVTQ | 360 |
| ETANLEYKDQ SGALNESFSD VEGYFVDDED FLMGEDVYTP GKEGDALRSM SNPEQFGQPA | 420 |
| HMKDYVFTEK DNGGVHTNSG IPNKAAYNVI QAIGKSKSEQ IYYRALTEYL TSNSNFKDCK | 480 |
| DALYQAAKDL YDEQTAEQVY EAWNEVGVE | 509 |
| (USA300HOU_2637-28-509) | |
| SEQ ID NO: 34 | |
| IDSKNKPANS DIKFEVTQKS DAVKALKELP KSENVKNIYQ DYAVTDVKTD KKGFTHYTLQ | 60 |
| PSVDGVHAPD KEVKVHADKS GKVVLINGDT DAKKVKPTNK VTLSKDDAAD KAFKAVKIDK | 120 |
| NKAKNLKDKV IKENKVEIDG DSNKYVYNVE LITVTPEISH WKVKIDAQTG EILEKMNLVK | 180 |
| EAAETGKGKG VLGDTKDINI NSIDGGFSLE DLTHQGKLSA FSENDQTGQA TLITNEDENF | 240 |
| VKDEQRAGVD ANYYAKQTYD YYKDTFGRES YDNQGSPIVS LTHVNNYGGQ DNRNNAAWIG | 300 |
| DKMIYGDGDG RTFTSLSGAN DVVAHELTHG VTQETANLEY KDQSGALNES FSDVFGYFVD | 360 |
| DEDFLMGEDV YTPGKEGDAL RSMSNPEQFG QPAHMKDYVF TEKDNGGVHT NSGIPNKAAY | 420 |
| NVIQAIGKSK SEQIYYRALT EYLTSNSNFK DCKDALYQAA KDLYDEQTAE QVYEAWNEVG | 480 |
| VE | 482 |
| (mc2716) | |
| SEQ ID NO: 35 | |
| IDSKNKPANS DIKFEVTQKS DAVKALKELP KSENVKNIYQ DYAVTDVKTD KKGFTHYTLQ | 60 |
| PSVDGVHAPD KEVKVHADKS GKVVLINGDT DAKKVKPTNK VTLSKDDAAD KAFKAVKIDK | 120 |
| NKAKNLKDKV IKENKVEIDG DSNKYVYNVE LITVTPEISH WKVKIDAQTG EILEKMNLVK | 180 |
| EAAETGKGKG VLGDTKDINI NSIDGGFSLE DLTHQGKLSA FSENDQTGQA TLITNEDENF | 240 |
| VKDEQRAGVD ANYYAKQTYD YYKDTFGRES YDNQGSPIVS LTHVNNYGGQ DNRNNAAWIG | 300 |
| DKMIYGDGDG RTFTSLSGAN DVVAHALTHG VTQETANLEY KDQSGALNES FSDVEGYFVD | 360 |
| DEDFLMGEDV YTPGKEGDAL RSMSNPEQFG QPAHMKDYVF TEKDNGGVHT NSGIPNKAAY | 420 |
| NVIQAIGKSK SEQIYYRALT EYLTSNSNFK DSKDALYQAA KDLYDEQTAE QVYEAWNEVG | 480 |
| VE | 482 |
| (2723; SAR2723) | |
| SEQ ID NO: 37 | |
| MPKNKILIYL LSTTLVLPTL VSPTAYADTP QKDTTAKTTS HDSKKSTDDE TSKDTTSKDI | 60 |
| DKADNNNTSN QDNNDKKVKT IDDSTSDSNN IIDFIYKNLP QTNINQLLTK NKYDDNYSLT | 120 |
| TLIQNLFNLN SDISDYEQPR NGEKSINDSN KNSDNSIKND TDTQSSKQDK ADNQKAPKSN | 180 |
| NTKPSTSNKQ PNSPKPTQPN QSNSQPASDD KVNQKSSSKD NQSMSDSALD SILDQYSEDA | 240 |
| KKTQKDYASQ SKKDKNEKSN TKNPQLPTQD ELKHKSKPAQ SFNNDVNQKD TRATSLFETD | 300 |
| PSISNNDDSG QFNVVDSKDT RQFVKSIAKD AHRIGQDNDI YASVMIAQAI LESDSGRSAL | 360 |
| AKSPNHNLFG IKGAFEGNSV PENTLEADGN QLYSINAGFR KYPSTKESLK DYSDLIKNGI | 420 |
| DGNRTIYKPT WKSEADSYKD ATSHLSKTYA TDPNYAKKLN SIIKHYQLTQ FDDERMPDLD | 480 |
| KYERSIKDYD DSSDEFKPER EVSDNMPYPH GQCTWYVYNR MKQFGTSISG DLGDAHNWNN | 540 |
| RAQYRDYQVS HTPKRHAAVV FEAGQFGADQ HYGHVAFVEK VNSDGSIVIS ESNVKGLGII | 600 |
| SHRTINAAAA EELSYITGK | 619 |
| (wt 2723-28-619) | |
| SEQ ID NO: 38 | |
| DTPQKDTTAK TTSHDSKKST DDETSKDTTS KDIDKADNNN TSNQDNNDKK VKTIDDSTSD | 60 |
| SNNIIDFIYK NLPQTNINQL LTKNKYDDNY SLTTLIQNLF NLNSDISDYE QPRNGEKSTN | 120 |
| DSNKNSDNSI KNDTDTQSSK QDKADNQKAP KSNNTKPSTS NKQPNSPKPT QPNQSNSQPA | 180 |
| SDDKVNQKSS SKDNQSMSDS ALDSILDQYS EDAKKTQKDY ASQSKKDKNE KSNTKNPQLP | 240 |
| TQDELKHKSK PAQSENNDVN QKDTRATSLF ETDPSISNND DSGQFNVVDS KDTRQFVKSI | 300 |
| AKDAHRIGQD NDIYASVMIA QAILESDSGR SALAKSPNHN LFGIKGAFEG NSVPENTLEA | 360 |
| DGNQLYSINA GFRKYPSTKE SLKDYSDLIK NGIDGNRTIY KPTWKSEADS YKDATSHLSK | 420 |
| TYATDPNYAK KLNSIIKHYQ LTQFDDERMP DLDKYERSIK DYDDSSDEFK PFREVSDNMP | 480 |
| YPHGQCTWYV YNRMKQFGTS ISGDLGDAHN WNNRAQYRDY QVSHTPKRHA AVVFEAGQFG | 540 |
| ADQHYGHVAF VEKVNSDGSI VISESNVKGL GIISHRTINA AAAEELSYIT GK | 592 |
| (mc2723) | |
| SEQ ID NO: 39 | |
| DTPQKDTTAK TTSHDSKKSN DDETSKDTTS KDIDKADNNN TSNQDNNDKK FKTIDDSTSD | 60 |
| SNNIIDFIYK NLPQTNINQL LTKNKYDDNY SLTTLIQNLF NLNSDISDYE QPRNGEKSTN | 120 |
| DSNKNSDNSI KNDTDTQSSK QDKADNQKAP KSNNTKPSTS NKQPNSPKPT QPNQSNSQPA | 180 |
| SDDKANQKSS SKDNQSMSDS ALDSILDQYS EDAKKTQKDY ASQSKKDKNE KSNTKNPQLP | 240 |
| TQDELKHKSK PAQSFNNDVN QKDTRATSLF ETDPSISNND DSGQFNVVDS KDTRQFVKSI | 300 |
| AKDAHRIGQD NDIYASVMIA QAILESDSGR SALAKSPNHN LFGIKGAFEG NSVPENTLQA | 360 |
| DGNKLYSINA GFRKYPSTKE SLKNYSDLIK NGIDGNRTIY KPTWKSEADS YKDATSHLSK | 420 |
| TYATDPNYAK KLNSIIKHYQ LTQFDDERMP DLDKYERSIK DYDDSSDEFK PFREVSDSMP | 480 |
| YPHGQSTWYV YNRMKQFGTS ISGDLGDAHN WNNRAQYRDY QVSHTPKRHA AVVFEAGQFG | 540 |
| ADQHYGHVAF VEKVNSDGSI VISESNVKGL GIISHRTINA AAAEELSYIT GK | 592 |
| (2753-291-680) | |
| SEQ ID NO: 42 | |
| KVAKQGQYKN QDPIVLVHGF NGFTDDINPS VLAHYWGGNK MNIRQDLEEN GYKAYEASIS | 60 |
| AFGSNYDRAV ELYYYIKGGR VDYGAAHAAK YGHERYGKTY EGIYKDWKPG QKVHLVGHSM | 120 |
| GGQTIRQLEE LLRNGSREEI EYQKKHGGEI SPLFKGNNDN MISSITTLGT PHNGTHASDL | 180 |
| AGNEALVRQI VFDIGKMFGN KNSRVDFGLA QWGLKQKPNE SYIDYVKRVK QSNLWKSKDN | 240 |
| GFYDLTREGA TDLNRKTSLN PNIVYKTYTG EATHKALNSD RQKADLNMFF PFVITGNLIG | 300 |
| KATEKEWREN DGLVSVISSQ HPENQAYTNA TDKIQKGIWQ VTPTKHDWDH VDFVGQDSSD | 360 |
| TVRTREELQD FWHHLADDLV KTEKVTDTKQ | 390 |
| (SAR0992) | |
| SEQ ID NO: 44 | |
| MDIGKKHVIP KSQYRRKRRE FFHNEDREEN LNQHQDKQNI DNTTSKKADK QIHKDSIDKH | 60 |
| ERFKNSLSSH LEQRNRDVNE NKAEESKSNQ DSKSAYNRDH YLTDDVSKKQ NSLDSVDQDT | 120 |
| EKSKYYEQNS EATLSTKSTD KVESTEMRKL SSDKNKVGHE EQHVLSKPSE HDKETRIDSE | 180 |
| SSRTDSDSSM QTEKIKKDSS DGNKSSNLKS EVISDKSNTV PKLSESDDEV NNQKPLTLPE | 240 |
| EQKLKRQQSQ NEQTKTYTYG DSEQNDKSNH ENDLSHHIPS ISDDKDNVMR ENHIVDDNPD | 300 |
| NDINTPSLSK TDDDRKLDEK IHVEDKHKQN ADSSETVGYQ SQSTASHRST EKRNISINDH | 360 |
| DKLNGQKTNT KTSANNNQKK ATSKLNKGRA TNNNYSDILK KFWMMYWPKL VILMGIIILI | 420 |
| VILNAIFNNV NKNDRMNDNN DADAQKYTTT MKNANNTVKS VVTVENETSK DSSLPKDKAS | 480 |
| QDEVGSGVVY KKSGDTLYIV TNAHVVGDKE NQKITFSNNK SVVGKVLGKD KWSDLAVVKA | 540 |
| TSSDSSVKEI AIGDSNNLVL GEPILVVGNP LGVDFKGTVT EGIISGLNRN VPIDEDKDNK | 600 |
| YDMLMKAFQI DASVNPGNSG GAVVNREGKL IGVVAAKISM PNVENMSFAI PVNEVQKIVK | 660 |
| DLETKGKIDY PDVGVKMKNI ASLNSFERQA VKLPGKVKNG VVVDQVDNNG LADQSGLKKG | 720 |
| DVITELDGKL LEDDLRFRQI IFSHKDDLKS ITAKIYRDGK EKEINIKLK | 769 |
| (m0992; SAR0992_S619A-428-769) | |
| SEQ ID NO: 45 | |
| NNVNKNDRMN DNNDADAQKY TTTMKNANNT VKSVVTVENE TSKDSSLPKD KASQDEVGSG | 60 |
| VVYKKSGDTL YIVTNAHVVG DKENQKITFS NNKSVVGKVL GKDKWSDLAV VKATSSDSSV | 120 |
| KEIAIGDSNN LVLGEPILVV GNPLGVDFKG TVTEGIISGL NRNVPIDEDK DNKYDMLMKA | 180 |
| FQIDASVNPG NAGGAVVNRE GKLIGVVAAK ISMPNVENMS FAIPVNEVQK IVKDLETKGK | 240 |
| IDYPDVGVKM KNIASLNSFE RQAVKLPGKV KNGVVVDQVD NNGLADQSGL KKGDVITELD | 300 |
| GKLLEDDLRF RQIIFSHKDD LKSITAKIYR DGKEKEINIK LK | 342 |
| (CHIM_m0992_0992_FL) | |
| SEQ ID NO: 46 | |
| NNVNKNDRMN DNNDADAQKY TTTMKNANNT VKSVVTVENE TSKDSSLPKD KASQDEVGSG | 60 |
| VVYKKSGDTL YIVTNAHVVG DKENQKITFS NNKSVVGKVL GKDKWSDLAV VKATSSDSSV | 120 |
| KEIAIGDSNN LVLGEPILVV GNPLGVDFKG TVTEGIISGL NRNVPIDEDK DNKYDMLMKA | 180 |
| FQIDASVNPG NAGGAVVNRE GKLIGVVAAK ISMPNVENMS FAIPVNEVQK IVKDLETKGK | 240 |
| IDYPDVGVKM KNIASLNSFE RQAVKLPGKV KNGVVVDQVD NNGLADQSGL KKGDVITELD | 300 |
| GKLLEDDLRF RQIIFSHKDD LKSITAKIYR DGKEKEINIK LKGSGGGAGS GGGAMDIGKK | 360 |
| HVIPKSQYRR KRREFFHNED REENLNQHQD KQNIDNTTSK KADKQIHKDS IDKHERFKNS | 420 |
| LSSHLEQRNR DVNENKAEES KSNQDSKSAY NRDHYLTDDV SKKQNSLDSV DQDTEKSKYY | 480 |
| EQNSEATLST KSTDKVESTE MRKLSSDKNK VGHEEQHVLS KPSEHDKETR IDSESSRTDS | 540 |
| DSSMQTEKIK KDSSDGNKSS NLKSEVISDK SNTVPKLSES DDEVNNQKPL TLPEEQKLKR | 600 |
| QQSQNEQTKT YTYGDSEQND KSNHENDLSH HIPSISDDKD NVMRENHIVD DNPDNDINTP | 660 |
| SLSKTDDDRK LDEKIHVEDK HKQNADSSET VGYQSQSTAS HRSTEKRNIS INDHDKLNGQ | 720 |
| KTNTKTSANN NQKKATSKLN KGRATNNNYS DILKKFWMMY WPK | 763 |
| (wt_SAR0280) | |
| SEQ ID NO: 47 | |
| MKKKNWIYAL IVTLIIIIAI VSMIFFVQTK YGDQSEKGSQ SVSNKNNKIH IAIVNEDQPT | 60 |
| TYNGKKVELG QAFIKRLANE KNYKFETVTR NVAESGLKNG GYQVMIVIPE NESKLAMQLD | 120 |
| AKTPSKISLQ YKTAVGQKEE VAKNTEKVVS NVLNDENKNL VEIYLTSIID NLHNAQKNVG | 180 |
| AIMTREHGVN SKESNYLLNP INDFPELFTD TLVNSISANK DITKWFQTYN KSLLSANSDT | 240 |
| FRVNTDYNVS TLIEKQNSLF DEHNTAMDKM LQDYKSQKDS VELDNYINAL KQMDSQIDQQ | 300 |
| SSMQDTGKEE YKQTVKENLD KLREIIQSQE SPFSKGMIED YRKQLTESLQ DELANNKDLQ | 360 |
| DALNSIKMNN AQFAENLEKQ LHDDIVKEPD SDTTFIYNMS KQDFIAAGLN EDEANKYEAI | 420 |
| VKEAKRYKNE YNLKKPLAEH INLTDYDNQV AQDTSSLIND GVKVQRTETI KSNDINQLTV | 480 |
| ATDPHFNFEG DIKINGKKYD IKDQSVQLDT SNKEYKVEVN GVAKLKKDAE KDFLKDKTMH | 540 |
| LQLLFGQANR QDEPNDKKAT SVVDVTLNHN LDGRLSKDAL SQQLSALSRF DAHYKMYTDT | 600 |
| KGREDKPFDN KRLIDMMVDQ VINDMESFKD DKVAVLHQID SMEENSDKLI DDILNNKKNT | 660 |
| TKNKEDISKL IDQLENVKKT FAEEPQEPKI DKGKNDEFNT MSSNLDKEIS RISEKSTQLL | 720 |
| SDTQESKTIA DSVSGQLNQL DNNVNKLHAT GRALGVRAND LNRQMAKNDK DNELFAKEFK | 780 |
| KVLQNSKDGD RQNQALKAFM SNPVQKKNLE NVLANNGNTD VISPTLFVLL MYLLSMITAY | 840 |
| IFYSYERAKG QMNFIKDDYS SKNNLWNNAI TSGVIGATGL VEGLIVGLIA MNKFHVLAGY | 900 |
| RAKFILMVIL TMMVFVLINT YLLRQVKSIG MFLMIAALGL YFVAMNNLKA AGQGVTNKIS | 960 |
| PLSYIDNMFF NYLNAEHPIG LALVILTVLV IIGFVLNMFI KHEKKERLI | 1009 |
| (0280-28-820; SAR0280-28-820) | |
| SEQ ID NO: 48 | |
| QTKYGDQSEK GSQSVSNKNN KIHIAIVNED QPTTYNGKKV ELGQAFIKRL ANEKNYKFET | 60 |
| VTRNVAESGL KNGGYQVMIV IPENFSKLAM QLDAKTPSKI SLQYKTAVGQ KEEVAKNTEK | 120 |
| VVSNVLNDEN KNLVEIYLTS IIDNLHNAQK NVGAIMTREH GVNSKESNYL LNPINDFPEL | 180 |
| FTDTLVNSIS ANKDITKWFQ TYNKSLLSAN SDTFRVNTDY NVSTLIEKQN SLFDEHNTAM | 240 |
| DKMLQDYKSQ KDSVELDNYI NALKQMDSQI DQQSSMQDTG KEEYKQTVKE NLDKLREIIQ | 300 |
| SQESPFSKGM IEDYRKQLTE SLQDELANNK DLQDALNSIK MNNAQFAENL EKQLHDDIVK | 360 |
| EPDSDTTFIY NMSKQDFIAA GLNEDEANKY EAIVKEAKRY KNEYNLKKPL AEHINLTDYD | 420 |
| NQVAQDTSSL INDGVKVQRT ETIKSNDINQ LTVATDPHFN FEGDIKINGK KYDIKDQSVQ | 480 |
| LDTSNKEYKV EVNGVAKLKK DAEKDFLKDK TMHLQLLFGQ ANRQDEPNDK KATSVVDVTL | 540 |
| NHNLDGRLSK DALSQQLSAL SRFDAHYKMY TDTKGREDKP FDNKRLIDMM VDQVINDMES | 600 |
| FKDDKVAVLH QIDSMEENSD KLIDDILNNK KNTTKNKEDI SKLIDQLENV KKTFAEEPQE | 660 |
| PKIDKGKNDE FNTMSSNLDK EISRISEKST QLLSDTQESK TIADSVSGQL NQLDNNVNKL | 720 |
| HATGRALGVR ANDLNRQMAK NDKDNELFAK EFKKVLQNSK DGDRQNQALK AFMSNPVQKK | 780 |
| NLENVLANNG NTD | 793 |
| (s0280; SAR0280-949-978) | |
| SEQ ID NO: 49 | |
| KAAGQGVINK ISPLSYIDNM FENYLNAEHP | 30 |
| (CHIM_LukE_mHla_H35L_FS) | |
| SEQ ID NO: 56 | |
| NTNIENIGDG AEVIKRTEDV SSKKWGVTQN VQFDFVKDKK YNKDALIVKM QGFINSRTSF | 60 |
| SDVKGSGYEL TKRMIWPFQY NIGLTTKDPN VSLINYLPKN KIETTDVGQT LGYNIGGNFQ | 120 |
| SAPSIGGNGS FNYSKTISYT QKSYVSEVDK QNSKSVKWGV KANEFVTPDG KKSAHDRYLF | 180 |
| VQSPNGPTGS AREYFAPDNQ LPPLVQSGEN PSFITTLSHE KGSSDTSEFE ISYGRNLDIT | 240 |
| YATLFPRTGI YAERKHNAFV NRNFVVRYEV NWKTHEIKVK GHNGSGGGAS ETEVSVRSAS | 300 |
| SDIGSNTTVK TGDLVTYDKE NGMLKKVFYS FIDDKNHNKK LLVIRTKGTI AGQYRVYSEE | 360 |
| GANKSGLAWP SAFKVQLQLP DNEVAQISDY YPRNSIDTKE YMSTLTYGEN GNVTGDDTGK | 420 |
| IGGLIGANVS IGHTLKYVQP DFKTILESPT DKKVGWKVIF NNMVNQNWGP YDRDSWNPVY | 480 |
| GNQLFMKTRN GSMKAADNFL DPNKASSLLS SGFSPDFATV ITMDRKASKQ QTNIDVIYER | 540 |
| VRDDYQLHWT STNWKGTNTK DKWIDRSSER YKIDWEKEEM TN | 582 |
| (CHIM_mc2716_mSpA_7_12_FS) | |
| SEQ ID NO: 57 | |
| IDSKNKPANS DIKFEVTQKS DAVKALKELP KSENVKNIYQ DYAVTDVKTD KKGFTHYTLQ | 60 |
| PSVDGVHAPD KEVKVHADKS GKVVLINGDT DAKKVKPTNK VTLSKDDAAD KAFKAVKIDK | 120 |
| NKAKNLKDKV IKENKVEIDG DSNKYVYNVE LITVTPEISH WKVKIDAQTG EILEKMNLVK | 180 |
| EAAETGKGKG VLGDTKDINI NSIDGGFSLE DLTHQGKLSA FSFNDQTGQA TLITNEDENF | 240 |
| VKDEQRAGVD ANYYAKQTYD YYKDTFGRES YDNQGSPIVS LTHVNNYGGQ DNRNNAAWIG | 300 |
| DKMIYGDGDG RTFTSLSGAN DVVAHALTHG VTQETANLEY KDQSGALNES FSDVFGYFVD | 360 |
| DEDFLMGEDV YTPGKEGDAL RSMSNPEQFG QPAHMKDYVF TEKDNGGVHT NSGIPNKAAY | 420 |
| NVIQAIGKSK SEQIYYRALT EYLTSNSNFK DSKDALYQAA KDLYDEQTAE QVYEAWNEVG | 480 |
| VEGSGGGAAQ VDEAQKNAFY QVLNMPNLNA DQRNGFIQSL KDAPAKSANV LGEAQKLNDS | 540 |
| QAPKADAQQN NVNKDQKSAF YEILNMPNLN EAQRNGFIQS LKDAPAKSTN VLGEAKKLNE | 600 |
| SQAPKADNNV NKEQKNAFYE ILNMPNLNEE QRNGFIQSLK DAPAKSANLL SEAKKLNESQ | 660 |
| APKADNKVNK EQKNAFYEIL HLPNLNEEQR NGFIQSLKDA PAKSANLLAE AKKLNDAQAP | 720 |
| KADNKVNKEQ KNAFYEILHL PNLTEEQRNG FIQSLKDAPA KSKEILAEAK KLNDAQAPK | 779 |
| (CHIM_0992_mSpA_7_12_FS) | |
| SEQ ID NO: 58 | |
| MDIGKKHVIP KSQYRRKRRE FFHNEDREEN LNQHQDKQNI DNTTSKKADK QIHKDSIDKH | 60 |
| ERFKNSLSSH LEQRNRDVNE NKAEESKSNQ DSKSAYNRDH YLTDDVSKKQ NSLDSVDQDT | 120 |
| EKSKYYEQNS EATLSTKSTD KVESTEMRKL SSDKNKVGHE EQHVLSKPSE HDKETRIDSE | 180 |
| SSRTDSDSSM QTEKIKKDSS DGNKSSNLKS EVISDKSNTV PKLSESDDEV NNQKPLTLPE | 240 |
| EQKLKRQQSQ NEQTKTYTYG DSEQNDKSNH ENDLSHHIPS ISDDKDNVMR ENHIVDDNPD | 300 |
| NDINTPSLSK TDDDRKLDEK IHVEDKHKQN ADSSETVGYQ SQSTASHRST EKRNISINDH | 360 |
| DKLNGQKTNT KTSANNNQKK ATSKLNKGRA TNNNYSDILK KFWMMYWPKG SGGGAAQVDE | 420 |
| AQKNAFYQVL NMPNLNADQR NGFIQSLKDA PAKSANVLGE AQKLNDSQAP KADAQQNNVN | 480 |
| KDQKSAFYEI LNMPNLNEAQ RNGFIQSLKD APAKSTNVLG EAKKLNESQA PKADNNVNKE | 540 |
| QKNAFYEILN MPNLNEEQRN GFIQSLKDAP AKSANLLSEA KKLNESQAPK ADNKVNKEQK | 600 |
| NAFYEILHLP NLNEEQRNGF IQSLKDAPAK SANLLAEAKK LNDAQAPKAD NKVNKEQKNA | 660 |
| FYEILHLPNL TEEQRNGFIQ SLKDAPAKSK EILAEAKKLN DAQAPK | 706 |
| (CHIM_2753_mc2723_FS) | |
| SEQ ID NO: 59 | |
| KVAKQGQYKN QDPIVLVHGF NGFTDDINPS VLAHYWGGNK MNIRQDLEEN GYKAYEASIS | 60 |
| AFGSNYDRAV ELYYYIKGGR VDYGAAHAAK YGHERYGKTY EGIYKDWKPG QKVHLVGHSM | 120 |
| GGQTIRQLEE LLRNGSREEI EYQKKHGGEI SPLFKGNNDN MISSITTLGT PHNGTHASDL | 180 |
| AGNEALVRQI VFDIGKMFGN KNSRVDFGLA QWGLKQKPNE SYIDYVKRVK QSNLWKSKDN | 240 |
| GFYDLTREGA TDLNRKTSLN PNIVYKTYTG EATHKALNSD RQKADLNMFF PFVITGNLIG | 300 |
| KATEKEWREN DGLVSVISSQ HPFNQAYTNA TDKIQKGIWQ VTPTKHDWDH VDFVGQDSSD | 360 |
| TVRTREELQD FWHHLADDLV KTEKVTDTKQ GSGGGADTPQ KDTTAKTTSH DSKKSNDDET | 420 |
| SKDTTSKDID KADNNNTSNQ DNNDKKFKTI DDSTSDSNNI IDFIYKNLPQ TNINQLLTKN | 480 |
| KYDDNYSLTT LIQNLFNLNS DISDYEQPRN GEKSTNDSNK NSDNSIKNDT DTQSSKQDKA | 540 |
| DNQKAPKSNN TKPSTSNKQP NSPKPTQPNQ SNSQPASDDK ANQKSSSKDN QSMSDSALDS | 600 |
| ILDQYSEDAK KTQKDYASQS KKDKNEKSNT KNPQLPTQDE LKHKSKPAQS FNNDVNQKDT | 660 |
| RATSLFETDP SISNNDDSGQ FNVVDSKDTR QFVKSIAKDA HRIGQDNDIY ASVMIAQAIL | 720 |
| ESDSGRSALA KSPNHNLFGI KGAFEGNSVP FNTLQADGNK LYSINAGFRK YPSTKESLKN | 780 |
| YSDLIKNGID GNRTIYKPTW KSEADSYKDA TSHLSKTYAT DPNYAKKLNS IIKHYQLTQF | 840 |
| DDERMPDLDK YERSIKDYDD SSDEFKPFRE VSDSMPYPHG QSTWYVYNRM KQFGTSISGD | 900 |
| LGDAHNWNNR AQYRDYQVSH TPKRHAAVVF EAGQFGADQH YGHVAFVEKV NSDGSIVISE | 960 |
| SNVKGLGIIS HRTINAAAAE ELSYITGK | 988 |
| (CHIM_0280_2753_FS) | |
| SEQ ID NO: 60 | |
| QTKYGDQSEK GSQSVSNKNN KIHIAIVNED QPTTYNGKKV ELGQAFIKRL ANEKNYKFET | 60 |
| VTRNVAESGL KNGGYQVMIV IPENFSKLAM QLDAKTPSKI SLQYKTAVGQ KEEVAKNTEK | 120 |
| VVSNVLNDEN KNLVEIYLTS IIDNLHNAQK NVGAIMTREH GVNSKFSNYL LNPINDFPEL | 180 |
| FTDTLVNSIS ANKDITKWFQ TYNKSLLSAN SDTFRVNTDY NVSTLIEKQN SLFDEHNTAM | 240 |
| DKMLQDYKSQ KDSVELDNYI NALKQMDSQI DQQSSMQDTG KEEYKQTVKE NLDKLREIIQ | 300 |
| SQESPFSKGM IEDYRKQLTE SLQDELANNK DLQDALNSIK MNNAQFAENL EKQLHDDIVK | 360 |
| EPDSDTTFIY NMSKQDFIAA GLNEDEANKY EAIVKEAKRY KNEYNLKKPL AEHINLTDYD | 420 |
| NQVAQDTSSL INDGVKVQRT ETIKSNDINQ LTVATDPHEN FEGDIKINGK KYDIKDQSVQ | 480 |
| LDTSNKEYKV EVNGVAKLKK DAEKDFLKDK TMHLQLLFGQ ANRQDEPNDK KATSVVDVTL | 540 |
| NHNLDGRLSK DALSQQLSAL SRFDAHYKMY TDTKGREDKP FDNKRLIDMM VDQVINDMES | 600 |
| FKDDKVAVLH QIDSMEENSD KLIDDILNNK KNTTKNKEDI SKLIDQLENV KKTFAEEPQE | 660 |
| PKIDKGKNDE FNTMSSNLDK EISRISEKST QLLSDTQESK TIADSVSGQL NQLDNNVNKL | 720 |
| HATGRALGVR ANDLNRQMAK NDKDNELFAK EFKKVLQNSK DGDRQNQALK AFMSNPVQKK | 780 |
| NLENVLANNG NTDGSGGGAK VAKQGQYKNQ DPIVLVHGEN GFTDDINPSV LAHYWGGNKM | 840 |
| NIRQDLEENG YKAYEASISA FGSNYDRAVE LYYYIKGGRV DYGAAHAAKY GHERYGKTYE | 900 |
| GIYKDWKPGQ KVHLVGHSMG GQTIRQLEEL LRNGSREEIE YQKKHGGEIS PLFKGNNDNM | 960 |
| ISSITTLGTP HNGTHASDLA GNEALVRQIV FDIGKMFGNK NSRVDFGLAQ WGLKQKPNES | 1020 |
| YIDYVKRVKQ SNLWKSKDNG FYDLTREGAT DLNRKTSLNP NIVYKTYTGE ATHKALNSDR | 1080 |
| QKADLNMFFP FVITGNLIGK ATEKEWREND GLVSVISSQH PFNQAYTNAT DKIQKGIWQV | 1140 |
| TPTKHDWDHV DFVGQDSSDT VRTREELQDF WHHLADDLVK TEKVTDTKQ | 1189 |
| (CHIM_0992_2753_FS) | |
| SEQ ID NO: 61 | |
| MDIGKKHVIP KSQYRRKRRE FFHNEDREEN LNQHQDKQNI DNTTSKKADK QIHKDSIDKH | 60 |
| ERFKNSLSSH LEQRNRDVNE NKAEESKSNQ DSKSAYNRDH YLTDDVSKKQ NSLDSVDQDT | 120 |
| EKSKYYEQNS EATLSTKSTD KVESTEMRKL SSDKNKVGHE EQHVLSKPSE HDKETRIDSE | 180 |
| SSRTDSDSSM QTEKIKKDSS DGNKSSNLKS EVISDKSNTV PKLSESDDEV NNQKPLTLPE | 240 |
| EQKLKRQQSQ NEQTKTYTYG DSEQNDKSNH ENDLSHHIPS ISDDKDNVMR ENHIVDDNPD | 300 |
| NDINTPSLSK TDDDRKLDEK IHVEDKHKQN ADSSETVGYQ SQSTASHRST EKRNISINDH | 360 |
| DKLNGQKTNT KTSANNNQKK ATSKLNKGRA TNNNYSDILK KFWMMYWPKG SGGGAKVAKQ | 420 |
| GQYKNQDPIV LVHGENGFTD DINPSVLAHY WGGNKMNIRQ DLEENGYKAY EASISAFGSN | 480 |
| YDRAVELYYY IKGGRVDYGA AHAAKYGHER YGKTYEGIYK DWKPGQKVHL VGHSMGGQTI | 540 |
| RQLEELLRNG SREEIEYQKK HGGEISPLEK GNNDNMISSI TTLGTPHNGT HASDLAGNEA | 600 |
| LVRQIVFDIG KMFGNKNSRV DFGLAQWGLK QKPNESYIDY VKRVKQSNLW KSKDNGFYDL | 660 |
| TREGATDLNR KTSLNPNIVY KTYTGEATHK ALNSDRQKAD LNMFFPFVIT GNLIGKATEK | 720 |
| EWRENDGLVS VISSQHPFNQ AYTNATDKIQ KGIWQVTPTK HDWDHVDFVG QDSSDTVRTR | 780 |
| EELQDFWHHL ADDLVKTEKV TDTKQ | 805 |
| (CHIM_mc2723_Hla_H35L_t_FS) | |
| SEQ ID NO: 62 | |
| DTPQKDTTAK TTSHDSKKSN DDETSKDTTS KDIDKADNNN TSNQDNNDKK FKTIDDSTSD | 60 |
| SNNIIDFIYK NLPQTNINQL LTKNKYDDNY SLTTLIQNLF NLNSDISDYE QPRNGEKSTN | 120 |
| DSNKNSDNSI KNDTDTQSSK QDKADNQKAP KSNNTKPSTS NKQPNSPKPT QPNQSNSQPA | 180 |
| SDDKANQKSS SKDNQSMSDS ALDSILDQYS EDAKKTQKDY ASQSKKDKNE KSNTKNPQLP | 240 |
| TQDELKHKSK PAQSENNDVN QKDTRATSLF ETDPSISNND DSGQFNVVDS KDTRQFVKSI | 300 |
| AKDAHRIGQD NDIYASVMIA QAILESDSGR SALAKSPNHN LFGIKGAFEG NSVPENTLQA | 360 |
| DGNKLYSINA GFRKYPSTKE SLKNYSDLIK NGIDGNRTIY KPTWKSEADS YKDATSHLSK | 420 |
| TYATDPNYAK KLNSIIKHYQ LTQFDDERMP DLDKYERSIK DYDDSSDEFK PFREVSDSMP | 480 |
| YPHGQSTWYV YNRMKQFGTS ISGDLGDAHN WNNRAQYRDY QVSHTPKRHA AVVFEAGQFG | 540 |
| ADQHYGHVAF VEKVNSDGSI VISESNVKGL GIISHRTINA AAAEELSYIT GKGSGGGADI | 600 |
| GSNTTVKTGD LVTYDKENGM LKKVFYSFID DKNHNKKLLV IRTKGTIAGQ YRVYSEEGAN | 660 |
| KSGLAWPSAF KVQLQLPDNE VAQISDYYPR NSIDTKEYMS TLTYGENGNV TGDDTGKIGG | 720 |
| LIGANVSIGH TLKYVQPDFK TILESPTDKK VGWKVIENNM VNQNWGPYDR DSWNPVYGNQ | 780 |
| LFMKTRNGSM KAADNFLDPN KASSLLSSGF SPDFATVITM DRKASKQQTN IDVIYERVRD | 840 |
| DYQLHWTSTN WKGTNTKDKW IDRSSERYKI DWEKEEMTN | 879 |
| (CHIM_mc2716_LukE_FS) | |
| SEQ ID NO: 63 | |
| IDSKNKPANS DIKFEVTQKS DAVKALKELP KSENVKNIYQ DYAVTDVKTD KKGFTHYTLQ | 60 |
| PSVDGVHAPD KEVKVHADKS GKVVLINGDT DAKKVKPTNK VTLSKDDAAD KAFKAVKIDK | 120 |
| NKAKNLKDKV IKENKVEIDG DSNKYVYNVE LITVTPEISH WKVKIDAQTG EILEKMNLVK | 180 |
| EAAETGKGKG VLGDTKDINI NSIDGGFSLE DLTHQGKLSA FSFNDQTGQA TLITNEDENF | 240 |
| VKDEQRAGVD ANYYAKQTYD YYKDTFGRES YDNQGSPIVS LTHVNNYGGQ DNRNNAAWIG | 300 |
| DKMIYGDGDG RTFTSLSGAN DVVAHALTHG VTQETANLEY KDQSGALNES FSDVFGYFVD | 360 |
| DEDFLMGEDV YTPGKEGDAL RSMSNPEQFG QPAHMKDYVE TEKDNGGVHT NSGIPNKAAY | 420 |
| NVIQAIGKSK SEQIYYRALT EYLTSNSNFK DSKDALYQAA KDLYDEQTAE QVYEAWNEVG | 480 |
| VEGSGGGANT NIENIGDGAE VIKRTEDVSS KKWGVTQNVQ FDFVKDKKYN KDALIVKMQG | 540 |
| FINSRTSFSD VKGSGYELTK RMIWPFQYNI GLTTKDPNVS LINYLPKNKI ETTDVGQTLG | 600 |
| YNIGGNFQSA PSIGGNGSEN YSKTISYTQK SYVSEVDKQN SKSVKWGVKA NEFVTPDGKK | 660 |
| SAHDRYLFVQ SPNGPTGSAR EYFAPDNQLP PLVQSGENPS FITTLSHEKG SSDTSEFEIS | 720 |
| YGRNLDITYA TLFPRTGIYA ERKHNAFVNR NFVVRYEVNW KTHEIKVKGH N | 771 |
| (CHIM_LukE_Hla_H35L_t_FS) | |
| SEQ ID NO: 64 | |
| NTNIENIGDG AEVIKRTEDV SSKKWGVTQN VQFDFVKDKK YNKDALIVKM QGFINSRTSF | 60 |
| SDVKGSGYEL TKRMIWPFQY NIGLTTKDPN VSLINYLPKN KIETTDVGQT LGYNIGGNFQ | 120 |
| SAPSIGGNGS FNYSKTISYT QKSYVSEVDK QNSKSVKWGV KANEFVTPDG KKSAHDRYLF | 180 |
| VQSPNGPTGS AREYFAPDNQ LPPLVQSGEN PSFITTLSHE KGSSDTSEFE ISYGRNLDIT | 240 |
| YATLFPRTGI YAERKHNAFV NRNFVVRYEV NWKTHEIKVK GHNGSGGGAD IGSNTTVKTG | 300 |
| DLVTYDKENG MLKKVFYSFI DDKNHNKKLL VIRTKGTIAG QYRVYSEEGA NKSGLAWPSA | 360 |
| FKVQLQLPDN EVAQISDYYP RNSIDTKEYM STLTYGENGN VTGDDTGKIG GLIGANVSIG | 420 |
| HTLKYVQPDF KTILESPTDK KVGWKVIENN MVNQNWGPYD RDSWNPVYGN QLFMKTRNGS | 480 |
| MKAADNFLDP NKASSLLSSG FSPDFATVIT MDRKASKQQT NIDVIYERVR DDYQLHWTST | 540 |
| NWKGTNTKDK WIDRSSERYK IDWEKEEMTN | 570 |
| (CHIM_mc2716_mSpA_10_12_FS) | |
| SEQ ID NO: 65 | |
| IDSKNKPANS DIKFEVTQKS DAVKALKELP KSENVKNIYQ DYAVTDVKTD KKGFTHYTLQ | 60 |
| PSVDGVHAPD KEVKVHADKS GKVVLINGDT DAKKVKPTNK VTLSKDDAAD KAFKAVKIDK | 120 |
| NKAKNLKDKV IKENKVEIDG DSNKYVYNVE LITVTPEISH WKVKIDAQTG EILEKMNLVK | 180 |
| EAAETGKGKG VLGDTKDINI NSIDGGFSLE DLTHQGKLSA FSFNDQTGQA TLITNEDENF | 240 |
| VKDEQRAGVD ANYYAKQTYD YYKDTFGRES YDNQGSPIVS LTHVNNYGGQ DNRNNAAWIG | 300 |
| DKMIYGDGDG RTFTSLSGAN DVVAHALTHG VTQETANLEY KDQSGALNES FSDVEGYFVD | 360 |
| DEDFLMGEDV YTPGKEGDAL RSMSNPEQFG QPAHMKDYVF TEKDNGGVHT NSGIPNKAAY | 420 |
| NVIQAIGKSK SEQIYYRALT EYLTSNSNFK DSKDALYQAA KDLYDEQTAE QVYEAWNEVG | 480 |
| VEGSGGGAAQ VDEAQKNAFY QVLNMPNLNA DQRNGFIQSL KDAPSQSAKV LGEAQKINDS | 540 |
| QAPKADAQQN NVNKDQKSAF YEILNMPNLN EAQRNGFIQS LKDAPSQSTK VLGEAKKLNE | 600 |
| SQAPKADNNV NKEQKNAFYE ILNMPNLNEE QRNGFIQSLK DAPSQSAKLL SEAKKLNESQ | 660 |
| APKADNKVNK EQKNAFYEIL HLPNLNEEQR NGFIQSLKDA PSQSAKLLAE AKKLNDAQAP | 720 |
| KADNKVNKEQ KNAFYEILHL PNLTEEQRNG FIQSLKDAPS VSKKILAEAK KLNDAQAPK | 779 |
| (CHIM_LukE_0280_FS) | |
| SEQ ID NO: 66 | |
| NTNIENIGDG AEVIKRTEDV SSKKWGVTQN VQFDFVKDKK YNKDALIVKM QGFINSRTSF | 60 |
| SDVKGSGYEL TKRMIWPFQY NIGLTTKDPN VSLINYLPKN KIETTDVGQT LGYNIGGNFQ | 120 |
| SAPSIGGNGS FNYSKTISYT QKSYVSEVDK QNSKSVKWGV KANEFVTPDG KKSAHDRYLF | 180 |
| VQSPNGPTGS AREYFAPDNQ LPPLVQSGEN PSFITTLSHE KGSSDTSEFE ISYGRNLDIT | 240 |
| YATLFPRTGI YAERKHNAFV NRNFVVRYEV NWKTHEIKVK GHNGSGGGAQ TKYGDQSEKG | 300 |
| SQSVSNKNNK IHIAIVNEDQ PTTYNGKKVE LGQAFIKRLA NEKNYKFETV TRNVAESGLK | 360 |
| NGGYQVMIVI PENFSKLAMQ LDAKTPSKIS LQYKTAVGQK EEVAKNTEKV VSNVLNDENK | 420 |
| NLVEIYLTSI IDNLHNAQKN VGAIMTREHG VNSKFSNYLL NPINDFPELF TDTLVNSISA | 480 |
| NKDITKWFQT YNKSLLSANS DTFRVNTDYN VSTLIEKQNS LFDEHNTAMD KMLQDYKSQK | 540 |
| DSVELDNYIN ALKQMDSQID QQSSMQDTGK EEYKQTVKEN LDKLREIIQS QESPFSKGMI | 600 |
| EDYRKQLTES LQDELANNKD LQDALNSIKM NNAQFAENLE KQLHDDIVKE PDSDTTFIYN | 660 |
| MSKQDFIAAG LNEDEANKYE AIVKEAKRYK NEYNLKKPLA EHINLTDYDN QVAQDTSSLI | 720 |
| NDGVKVQRTE TIKSNDINQL TVATDPHENF EGDIKINGKK YDIKDQSVQL DTSNKEYKVE | 780 |
| VNGVAKLKKD AEKDFLKDKT MHLQLLFGQA NRQDEPNDKK ATSVVDVTLN HNLDGRLSKD | 840 |
| ALSQQLSALS RFDAHYKMYT DTKGREDKPF DNKRLIDMMV DQVINDMESF KDDKVAVLHQ | 900 |
| IDSMEENSDK LIDDILNNKK NTTKNKEDIS KLIDQLENVK KTFAEEPQEP KIDKGKNDEF | 960 |
| NTMSSNLDKE ISRISEKSTQ LLSDTQESKT IADSVSGQLN QLDNNVNKLH ATGRALGVRA | 1020 |
| NDLNRQMAKN DKDNELFAKE FKKVLQNSKD GDRQNQALKA FMSNPVQKKN LENVLANNGN | 1080 |
| (CHIM_0992_m0992_FS) | |
| SEQ ID NO: 67 | |
| MDIGKKHVIP KSQYRRKRRE FFHNEDREEN LNQHQDKQNI DNTTSKKADK QIHKDSIDKH | 60 |
| ERFKNSLSSH LEQRNRDVNE NKAEESKSNQ DSKSAYNRDH YLTDDVSKKQ NSLDSVDQDT | 120 |
| EKSKYYEQNS EATLSTKSTD KVESTEMRKL SSDKNKVGHE EQHVLSKPSE HDKETRIDSE | 180 |
| SSRTDSDSSM QTEKIKKDSS DGNKSSNLKS EVISDKSNTV PKLSESDDEV NNQKPLTLPE | 240 |
| EQKLKRQQSQ NEQTKTYTYG DSEQNDKSNH ENDLSHHIPS ISDDKDNVMR ENHIVDDNPD | 300 |
| NDINTPSLSK TDDDRKLDEK IHVEDKHKQN ADSSETVGYQ SQSTASHRST EKRNISINDH | 360 |
| DKLNGQKTNT KTSANNNQKK ATSKLNKGRA TNNNYSDILK KFWMMYWPKG SGGGADIGSN | 420 |
| TTVKTGDLVT YDKENGMLKK VFYSFIDDKN HNKKLLVIRT KGTIAGQYRV YSEEGANKSG | 480 |
| LAWPSAFKVQ LQLPDNEVAQ ISDYYPRNSI DTKEYMSTLT YGENGNVTGD DTGKIGGLIG | 540 |
| ANVSIGHTLK YVQPDFKTIL ESPTDKKVGW KVIFNNMVNQ NWGPYDRDSW NPVYGNQLFM | 600 |
| KTRNGSMKAA DNFLDPNKAS SLLSSGFSPD FATVITMDRK ASKQQTNIDV IYERVRDDYQ | 660 |
| LHWTSTNWKG TNTKDKWIDR SSERYKIDWE KEEMTN | 696 |
| (CHIM_0992_Hla_H35L_t_FS) | |
| SEQ ID NO: 68 | |
| NNVNKNDRMN DNNDADAQKY TTTMKNANNT VKSVVTVENE TSKDSSLPKD KASQDEVGSG | 60 |
| VVYKKSGDTL YIVTNAHVVG DKENQKITFS NNKSVVGKVL GKDKWSDLAV VKATSSDSSV | 120 |
| KEIAIGDSNN LVLGEPILVV GNPLGVDFKG TVTEGIISGL NRNVPIDEDK DNKYDMLMKA | 180 |
| FQIDASVNPG NAGGAVVNRE GKLIGVVAAK ISMPNVENMS FAIPVNEVQK IVKDLETKGK | 240 |
| IDYPDVGVKM KNIASLNSFE RQAVKLPGKV KNGVVVDQVD NNGLADQSGL KKGDVITELD | 300 |
| GKLLEDDLRF RQIIFSHKDD LKSITAKIYR DGKEKEINIK LKGSGGGAGS GGGAMDIGKK | 360 |
| HVIPKSQYRR KRREFFHNED REENLNQHQD KQNIDNTTSK KADKQIHKDS IDKHERFKNS | 420 |
| LSSHLEQRNR DVNENKAEES KSNQDSKSAY NRDHYLTDDV SKKQNSLDSV DQDTEKSKYY | 480 |
| EQNSEATLST KSTDKVESTE MRKLSSDKNK VGHEEQHVLS KPSEHDKETR IDSESSRTDS | 540 |
| DSSMQTEKIK KDSSDGNKSS NLKSEVISDK SNTVPKLSES DDEVNNQKPL TLPEEQKLKR | 600 |
| QQSQNEQTKT YTYGDSEQND KSNHENDLSH HIPSISDDKD NVMRENHIVD DNPDNDINTP | 660 |
| SLSKTDDDRK LDEKIHVEDK HKQNADSSET VGYQSQSTAS HRSTEKRNIS INDHDKLNGQ | 720 |
| KTNTKTSANN NQKKATSKLN KGRATNNNYS DILKKFWMMY WPK | 763 |
| (CHIM_LukE_mSpA_10_12_FS) | |
| SEQ ID NO: 82 | |
| NTNIENIGDG AEVIKRTEDV SSKKWGVTQN VQFDFVKDKK YNKDALIVKM QGFINSRTSF | 60 |
| SDVKGSGYEL TKRMIWPFQY NIGLTTKDPN VSLINYLPKN KIETTDVGQT LGYNIGGNFQ | 120 |
| SAPSIGGNGS FNYSKTISYT QKSYVSEVDK QNSKSVKWGV KANEFVTPDG KKSAHDRYLF | 180 |
| VQSPNGPTGS AREYFAPDNQ LPPLVQSGEN PSFITTLSHE KGSSDTSEFE ISYGRNLDIT | 240 |
| YATLFPRTGI YAERKHNAFV NRNFVVRYEV NWKTHEIKVK GHNGSGGGAA QVDEAQKNAF | 300 |
| YQVLNMPNLN ADQRNGFIQS LKDAPSQSAK VLGEAQKIND SQAPKADAQQ NNVNKDQKSA | 360 |
| FYEILNMPNL NEAQRNGFIQ SLKDAPSQST KVLGEAKKLN ESQAPKADNN VNKEQKNAFY | 420 |
| EILNMPNLNE EQRNGFIQSL KDAPSQSAKL LSEAKKLNES QAPKADNKVN KEQKNAFYEI | 480 |
| LHLPNLNEEQ RNGFIQSLKD APSQSAKLLA EAKKLNDAQA PKADNKVNKE QKNAFYEILH | 540 |
| LPNLTEEQRN GFIQSLKDAP SVSKKILAEA KKLNDAQAPK | 580 |
| (CHIM_mc2723_mSpA_10_12_FS) | |
| SEQ ID NO: 83 | |
| DTPQKDTTAK TTSHDSKKSN DDETSKDTTS KDIDKADNNN TSNQDNNDKK FKTIDDSTSD | 60 |
| SNNIIDFIYK NLPQTNINQL LTKNKYDDNY SLTTLIQNLF NLNSDISDYE QPRNGEKSTN | 120 |
| DSNKNSDNSI KNDTDTQSSK QDKADNQKAP KSNNTKPSTS NKQPNSPKPT QPNQSNSQPA | 180 |
| SDDKANQKSS SKDNQSMSDS ALDSILDQYS EDAKKTQKDY ASQSKKDKNE KSNTKNPQLP | 240 |
| TQDELKHKSK PAQSENNDVN QKDTRATSLF ETDPSISNND DSGQFNVVDS KDTRQFVKSI | 300 |
| AKDAHRIGQD NDIYASVMIA QAILESDSGR SALAKSPNHN LFGIKGAFEG NSVPENTLQA | 360 |
| DGNKLYSINA GFRKYPSTKE SLKNYSDLIK NGIDGNRTIY KPTWKSEADS YKDATSHLSK | 420 |
| TYATDPNYAK KLNSIIKHYQ LTQFDDERMP DLDKYERSIK DYDDSSDEFK PFREVSDSMP | 480 |
| YPHGQSTWYV YNRMKQFGTS ISGDLGDAHN WNNRAQYRDY QVSHTPKRHA AVVFEAGQFG | 540 |
| ADQHYGHVAF VEKVNSDGSI VISESNVKGL GIISHRTINA AAAEELSYIT GKGSGGGAAQ | 600 |
| VDEAQKNAFY QVLNMPNLNA DQRNGFIQSL KDAPSQSAKV LGEAQKINDS QAPKADAQQN | 660 |
| NVNKDQKSAF YEILNMPNLN EAQRNGFIQS LKDAPSQSTK VLGEAKKLNE SQAPKADNNV | 720 |
| NKEQKNAFYE ILNMPNLNEE QRNGFIQSLK DAPSQSAKLL SEAKKLNESQ APKADNKVNK | 780 |
| EQKNAFYEIL HLPNLNEEQR NGFIQSLKDA PSQSAKLLAE AKKLNDAQAP KADNKVNKEQ | 840 |
| KNAFYEILHL PNLTEEQRNG FIQSLKDAPS VSKKILAEAK KLNDAQAPK | 889 |
| (CHIM_0992_mSpA_10_12_FS) | |
| SEQ ID NO: 84 | |
| MDIGKKHVIP KSQYRRKRRE FFHNEDREEN LNQHQDKQNI DNTTSKKADK QIHKDSIDKH | 60 |
| ERFKNSLSSH LEQRNRDVNE NKAEESKSNQ DSKSAYNRDH YLTDDVSKKQ NSLDSVDQDT | 120 |
| EKSKYYEQNS EATLSTKSTD KVESTEMRKL SSDKNKVGHE EQHVLSKPSE HDKETRIDSE | 180 |
| SSRTDSDSSM QTEKIKKDSS DGNKSSNLKS EVISDKSNTV PKLSESDDEV NNQKPLTLPE | 240 |
| EQKLKRQQSQ NEQTKTYTYG DSEQNDKSNH ENDLSHHIPS ISDDKDNVMR ENHIVDDNPD | 300 |
| NDINTPSLSK TDDDRKLDEK IHVEDKHKQN ADSSETVGYQ SQSTASHRST EKRNISINDH | 360 |
| DKLNGQKTNT KTSANNNQKK ATSKLNKGRA TNNNYSDILK KFWMMYWPKG SGGGAAQVDE | 420 |
| AQKNAFYQVL NMPNLNADQR NGFIQSLKDA PSQSAKVLGE AQKLNDSQAP KADAQQNNVN | 480 |
| KDQKSAFYEI LNMPNLNEAQ RNGFIQSLKD APSQSTKVLG EAKKLNESQA PKADNNVNKE | 540 |
| QKNAFYEILN MPNLNEEQRN GFIQSLKDAP SQSAKLLSEA KKLNESQAPK ADNKVNKEQK | 600 |
| NAFYEILHLP NLNEEQRNGF IQSLKDAPSQ SAKLLAEAKK LNDAQAPKAD NKVNKEQKNA | 660 |
| FYEILHLPNL TEEQRNGFIQ SLKDAPSVSK KILAEAKKLN DAQAPK | 706 |
| (CHIM_LukE_mSpA_7_12_FS) | |
| SEQ ID NO: 85 | |
| NTNIENIGDG AEVIKRTEDV SSKKWGVTQN VQFDEVKDKK YNKDALIVKM QGFINSRTSF | 60 |
| SDVKGSGYEL TKRMIWPFQY NIGLTTKDPN VSLINYLPKN KIETTDVGQT LGYNIGGNFQ | 120 |
| SAPSIGGNGS FNYSKTISYT QKSYVSEVDK QNSKSVKWGV KANEFVTPDG KKSAHDRYLF | 180 |
| VQSPNGPTGS AREYFAPDNQ LPPLVQSGEN PSFITTLSHE KGSSDTSEFE ISYGRNLDIT | 240 |
| YATLFPRTGI YAERKHNAFV NRNFVVRYEV NWKTHEIKVK GHNGSGGGAA QVDEAQKNAF | 300 |
| YQVLNMPNLN ADQRNGFIQS LKDAPAKSAN VLGEAQKIND SQAPKADAQQ NNVNKDQKSA | 360 |
| FYEILNMPNL NEAQRNGFIQ SLKDAPAKST NVLGEAKKLN ESQAPKADNN VNKEQKNAFY | 420 |
| EILNMPNLNE EQRNGFIQSL KDAPAKSANL LSEAKKLNES QAPKADNKVN KEQKNAFYEI | 480 |
| LHLPNLNEEQ RNGFIQSLKD APAKSANLLA EAKKLNDAQA PKADNKVNKE QKNAFYEILH | 540 |
| LPNLTEEQRN GFIQSLKDAP AKSKEILAEA KKLNDAQAPK | 580 |

Table 1 gives an overview of all of the above amino acid sequences by referring to their nomenclature and any alternative nomenclature.

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the scope of the appended claims will occur to those skilled in the art.

TABLE 1
Overview of the protein constructs of the present invention.

| SEQ ID NO: | Nomenclature | Alternative nomenclature |
|---|---|---|
| 1 | Hla - wild type full length | |
| 2 | Hla_wt-27-319 | |
| 3 | Hla_t-39-319 | |
| 4 | mHla-27-319 | |
| 5 | Hla_H35L-27-319 | HlaH35L_27-319 |
| 6 | Hla_H35L_t-39-319 | Hla_H35L-39-319 |
| 7 | mHla_H35L-27-319 | |
| 15 | SpA_wild_type_full_length | |
| 16 | SpA_wt_t | SpA_wt-49-339 |
| 17 | SpA_10_12 | SpA_mut_10_12/mSpA_10_12 |
| 18 | SpA_7_12 | SpA_mut_7_12/mSpA_7_12 |
| 19 | SpA_4_12 | SpA_mut_4_12/mSpA_4_12 |
| 20 | SpA_3_12 | SpA_mut_3_12/mSpA_3_12 |
| 21 | SpA_10_15 | SpA_mut_10_15/mSpA_10_15 |
| 29 | wt_LukE | |
| 30 | LukE-29-311 | |
| 33 | USA300HOU_2637 | |
| 34 | USA300HOU_2637-28-509 | |
| 35 | mc2716 | USA300HOU_2637_E353A_C478S-28-509/ |
| | | USA300HOU_2637_E353A-28-509 |
| 37 | 2723; SAR2723 | |
| 38 | wt_2723-28-619 | |
| 39 | mc2723 | SAR2723_E386Q_D411N_C513S-28-619 |
| 42 | 2753-291-680 | SAR2753-291-680 |
| 44 | SAR0992 | |
| 45 | m0992; SAR0992_S619A-418-759 | |
| 46 | CHIM_m0992_0992_FL | |
| 47 | wt_SAR0280 | |
| 48 | 0280-28-820; SAR0280-28-820 | |
| 49 | s0280; SAR0280-949-978 | |
| 56 | CHIM_LukE_mHla_H35L_FS | |
| 57 | CHIM_mc2716_mSpA_7_12_FS | |
| 58 | CHIM_0992_mSpA_7_12_FS | |
| 59 | CHIM_2753_mc2723_FS | |
| 60 | CHIM_0280_2753_FS | |
| 61 | CHIM_0992_2753_FS | |
| 62 | CHIM_mc2723_Hla_H35L_t_FS | |
| 63 | CHIM_mc2716_LukE_FS | |
| 64 | CHIM_LukE_Hla_H35L_t_FS | |
| 65 | CHIM_mc2716_mSpA_10_12_FS | |
| 66 | CHIM_LukE_0280_FS | |
| 67 | CHIM_0992_m0992_FS | |
| 68 | CHIM 0992 Hla H35L t FS | |

### Example 1: Functional Assessment of Staphylococcal Protein Variants

### 1.1 SpA Variants

A range of different SpA variants were tested for their ability to bind to Ig and von Willebrand factor (vWF).

Using ELISA, the following SpA variants were found in ELISA not to bind to any of mouse IgG, rabbit IgG, and mouse IgM from whole sera, purified human and mouse IgG, and vWF:

TABLE 2
SpA variants found to have abrogated binding to Ig and vWF. Two or three mutations
were made in each Fab binding site and two mutations in each Fc binding site of the
domains E, D, A, B, and C. In the nomenclature SpA_X_Y, X is a number referring to
a specific combination of mutations in the Fab binding site, and Y is a number referring
to a specific combination of mutations in the Fc binding site.

| SpA variant | | |
|---|---|---|
| (mutation Fab_Fc) | Mutations in Fab binding sites | Mutations in Fc binding sites |
| SpA_10_12 | (D35A, N41K), (D96A, N102K), | (H3V, Q8K), (F64V, Q69K), |
| | (D154A, N160K), (D212A, | (F122V, Q127K), (F180V, |
| | N218K), (D270A, E276K) | Q185K), (F238V, Q243K) |
| SpA_7_12 | (D35A, S37A, Q38K), (D96A, | (H3V, Q8K), (F64V, Q69K), |
| | S98A, Q99K), (D154A, S156A, | (F122V, Q127K), (F180V, |
| | Q157K), (D212A, S214A, | Q185K), (F238V, Q243K) |
| | Q215K), (D270A, S272A, V273K) | |
| SpA_4_12 | (D34R, S37R), (D95R, S98R), | (H3V, Q8K), (F64V, Q69K), |
| | (D153R, S156R), (D211R, | (F122V, Q127K), (F180V, |
| | S214R), (D269R, S272R) | Q185K), (F238V, Q243K) |
| SpA_3_12 | (D35R, S37R), (D96R, S98R), | (H3V, Q8K), (F64V, Q69K), |
| | (D154R, S156R), (D212R, | (F122V, Q127K), (F180V, |
| | S214R), (D270R, S272R) | Q185K), (F238V, Q243K) |
| SpA_10_15 | (D35A, N41K), (D96A, N102K), | (Q8K, N9K), (Q69K, S70K), |
| | (D154A, N160K), (D212A, | (Q127K, N128K), (Q185K, |
| | N218K), (D270A, E276K) | N186K), (Q243K, N244K) |

More detailed data relating to the Ig binding abilities of SpA variants is given in the following.

### Igg Affinity Chromatography

To evaluate the different SpA constructs' ability not to bind human IgG, based on disruptive mutations in SpA, an affinity chromatography assay was set up. The principle of the assay was to immobilize the His-tagged SpA proteins (neutralizing the his-tag) on the chromatography column and then add with purified human IgG (neutralizing the his-tag of SpA variant constructs prior to test of human-IgG-SpA binding). Bound material was then eluted and run on an SDS-PAGE gel to visualize IgG protein binding.

The protocol was adopted from Kim et al., 2010; 2015 with minor modifications. Purified His-tagged SpA proteins (100 μg in 200 μL (0.5 μg/μL)) were immobilized on Ni-NTA agarose (100 μL, Qiagen #30230, Lot 151029515) for 20 min at room temperature (RT), washed over 6 CV or 10 CV with Binding Buffer (50 mM Tris, 150 mM NaCl, pH 7.5), and incubated with human IgG (100 μg in 200 μL (0.5 μg/μL), Sigma #I2511-10MG, lot O48M4868V) for 20 min at RT. Following washing over 20 CV or 10 CV with binding buffer supplemented with 30 mM imidazole, bound proteins were eluted over 5 CV with binding buffer supplemented with 500 mM imidazole and analyzed by SDS-PAGE (10 μL/fraction, 2.5 μg of starting material) (any kDa Criterion TGX Stain Free Protein Gel and Pro Blue Safe Stain, Giotto Biotech).

Results from the human IgG affinity chromatography showed that SpA_WT-49-339 (wild type (WT) full length SpA) (SEQ ID NO: 16) binds the entire amount of loaded IgG, whereas immobilized SpA_{KKAA}-49-339 mutant and several SpA mutant variants such as the SpA_mut_7_12 (SEQ ID NO: 18) and SpA_mut_10_12 (SEQ ID NO: 17), did not bind human IgG (Table 3).

TABLE 3
Human IgG binding ability of SpA mutant variants.

| SpA variant | Binding to IgG |
|---|---|
| SpA-WT-49-339 | Yes |
| SpA_{KKAA}-49-339 | No |
| SpA_mut_3_12 | No |
| SpA_mut_4_12 | No |
| SpA_mut_7_12 | No |
| SpA_mut_10_12 | No |
| SpA_mut_10_15 | No |

### Direct ELISA

To evaluate the different SpA mutant construct's ability to bind to a broad range of immunoglobulins, direct ELISA was applied to evaluate binding ability of human IgG, human vWF, mouse IgG and IgM, and rabbit IgG. The protocol described in Kim et al., 2015 was followed with few adjustments depending on the Ig evaluated in the assay.

To assess inhibition of mouse IgG binding to SpA mutant variants, the protocol described in in Kim et al., 2015 were used with following adjustments: Shortly, wells were coated with 300 ng of proteins/well diluted in PBS, pH 7.6 overnight at 4° C. Five-fold serial dilution of either purified mouse IgG (Sigma-Aldrich, #I8765-10MG, lot SLBW5857) starting from 40 ng/well, or a five-fold serial dilution of unspecific mouse sera starting from 1:1,000, was added to the plates. 1:5,000 or 1:2,000 dilution, respectively, of goat anti-mouse IgG antibody conjugated with alkaline phosphatase (Sigma-Aldrich, A3562, lot SLBK6489V) was added to the plate. As substrate, 3 mg/mL p-nitrophenyl phosphate (p-NPP; Sigma-Aldrich, P5869-25CAP, lot SLBV2576) in diethanolamine (DEA, pH 9.2; Sigma-Aldrich, D8885-500 g, lot MKCB9120) was used. The absorbance at 405 nm was read after 30 or 55 minutes of reaction, respectively, using a Tecan Infinite M200Pro device.

To assess inhibition of purified human IgG binding to SpA mutant variants, the protocol described in in Kim et al., 2015 were used with following adjustments: Shortly, wells were coated with 300 ng of SpA mutant proteins/well diluted in PBS, pH 7.6 overnight at 4° C. Five-fold dilution of human IgG (Sigma-Aldrich, #I2511-10MG, O48M4868V) starting from 40 ng/well was added to the plate, 1:10,000 goat anti-human IgG conjugated alkaline phosphatase (Sigma-Aldrich, #A1543-1ML, lot 029M4838V) was used as secondary antibody, and absorbance at 405 nm was read after 37 min of reaction using a Tecan Infinite M200Pro device.

To assess inhibition of unspecific rabbit IgG binding to SpA mutant variants, the protocol described in in Kim et al., 2015 were used with following adjustments: Shortly, wells were coated with 300 ng of SpA mutant proteins/well diluted in PBS, pH 7.6 overnight at 4° C. Three-fold dilution of unspecific rabbit sera starting from 1:1,000 dilution was added to the plate, 1:10,000 goat anti-rabbit IgG conjugated alkaline phosphatase (Sigma-Aldrich, A3687) was used as secondary antibody, and absorbance at 405 nm was read after 55 min of reaction using a Tecan Infinite M200Pro device.

To assess inhibition of unspecific mouse IgM binding to SpA mutant variants, the protocol described in in Kim et al., 2015 were used with following adjustments: Shortly, wells were coated with 300 ng of SpA mutant proteins/well diluted in PBS, pH 7.6 overnight at 4° C. Three-fold dilution of unspecific mouse sera starting from 1:1,000 dilution was added to the plate, 1:2,000 goat anti-mouse IgM conjugated alkaline phosphatase (Southern Biotech N1021-04, lot C7107RL27Z) was used as secondary antibody, and absorbance at 405 nm was read after 108 min of reaction using a Tecan Infinite M200Pro device.

To assess inhibition of unspecific human von Willebrand Factor (vWF) binding to SpA mutant variants, the protocol described in in Kim et al., 2015 were used with following adjustments: 300 ng of SpA mutant variant proteins per well diluted in PBS (pH 7.6) was coated in the 96-well plates overnight at 4° C. To each well, 300 ng of human vWF (Invitrogen #RP-43132, lot UE2767197) in 1% bovine serum albumin (BSA) was added and incubated for 1 hour. The sheep anti-human vWF antibody (Bio-Rad, #AHP062, lot 148125) was diluted 1:5,000 and incubated for 45 min. The donkey anti-sheep IgG conjugated with peroxidase (Sigma-Aldrich A3414-1 mL, lot SLBV6847) secondary antibody was diluted 1:30,000 and incubated for 45 min. As substrate, TMB (3,3′,5,5′-Tetramethylbenzidine, Biolegend, #421101, lot B304351) was used according to manufacturer's protocol. Stop solution, 1M H₃PO₄ (Sigma-Aldrich), was used. The absorbance at 405 nm was read after 45 min of reaction using a Tecan Infinite M200Pro device.

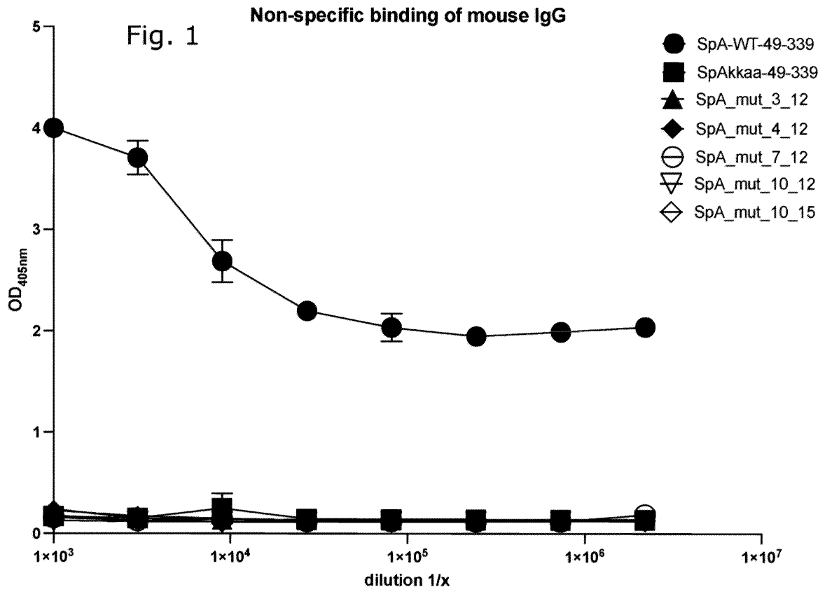

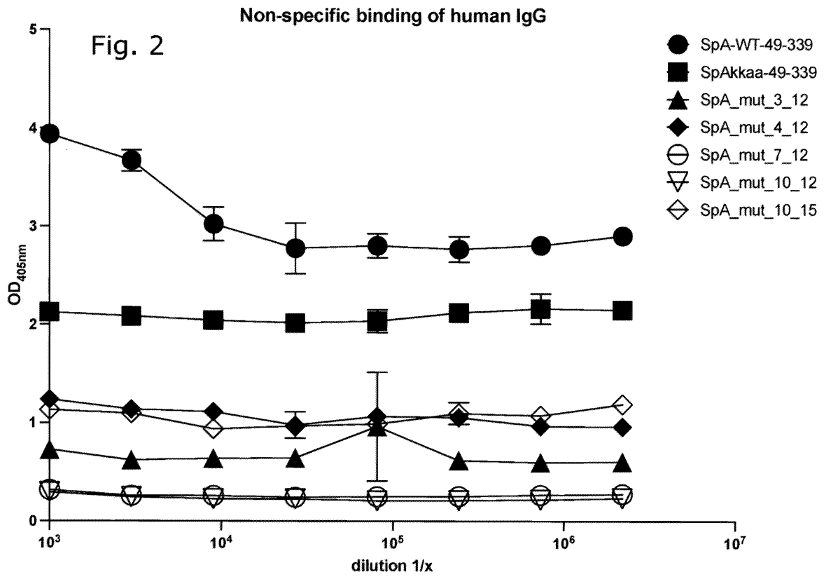

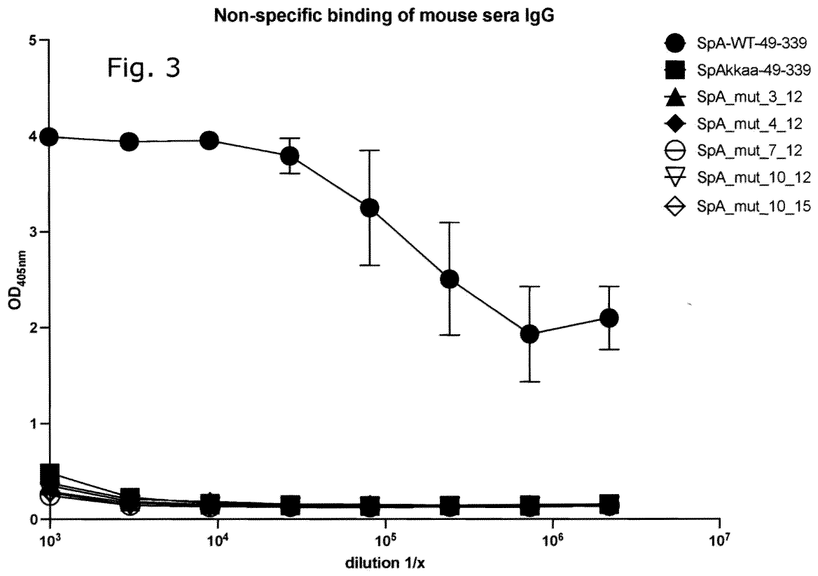

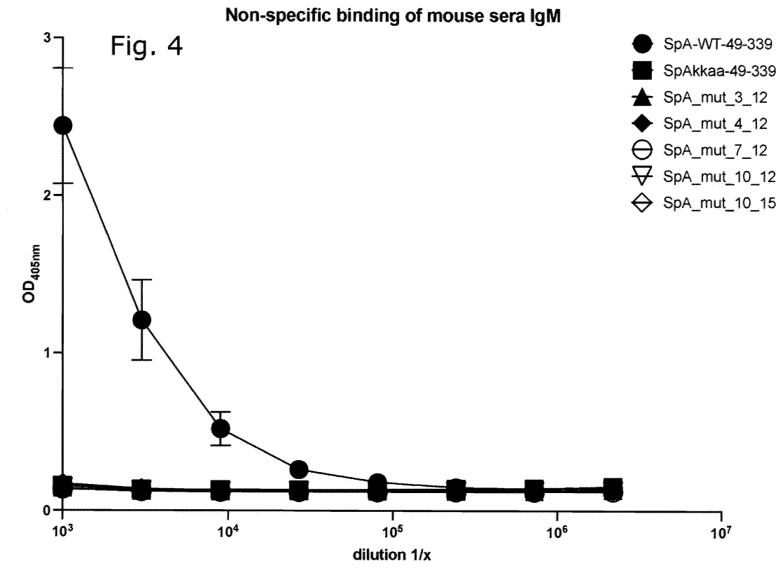

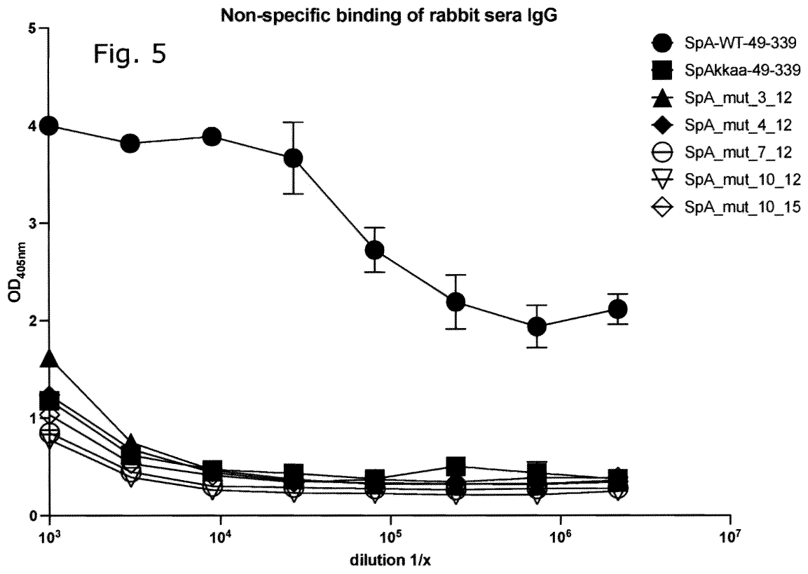

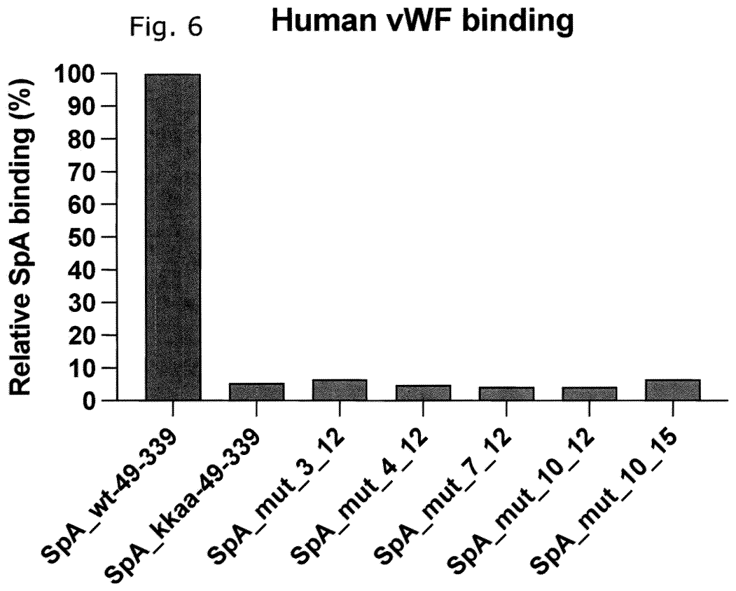

Results compiled in Table 4 and FIG. 1-6 show that the SpA_WT-49-339 (SEQ ID NO: 16) protein was able to bind both human IgG, mouse IgG and IgM, rabbit IgG and human vWF. The SpA_{KKAA}-49-339 mutant did not bind the mouse IgM, IgG's from mice and rabbits, nor vWF but did bind human IgG. SpA mutant variants SpA_mut_7_12 (SEQ ID NO: 18) and SpA_mut_10_12 (SEQ ID NO:17) were not able to bind human IgG, mouse IgG, mouse IgM, rabbit IgM nor vWF. The SpA mutant variants SpA_mut_3_12 (SEQ ID NO: 20), SpA_mut_4_12 (SEQ ID NO: 19) and SpA_mut_10_15 (SEQ ID NO: 21) did bind to human IgG, but could not bind to mouse IgG, mouse IgM, rabbit IgG nor vWF.

TABLE 4
Overview of SpA variants capability to bind IgG and
IgM from various animal sources as well as human vWF.

| | Purified | Purified | Unspecific | Unspecific | Unspecific | |
|---|---|---|---|---|---|---|
| | Mouse | Human | Mouse sera | Mouse sera | Rabbit sera | |
| SpA variants | IgG | IgG | IgG | IgM | IgG | vWF |
| SpA-WT-49-339 | Yes | Yes | Yes | Yes | Yes | Yes |
| SpAKKAA-49-339 | No | No | No | No | No | No |
| SpA_mut_3_12 | No | Yes | No | No | No | No |
| SpA_mut_4_12 | No | Yes | No | No | No | No |
| SpA_mut_7_12 | No | No | No | No | No | No |
| SpA_mut_10_12 | No | No | No | No | No | No |
| SpA_mut_10_15 | No | Yes | No | No | No | No |

### Competitive ELISA

Competitive ELISA was applied to address the blocking of SpA:IgG interaction by SpA variant polyclonal immune sera. To assess inhibition with chicken IgY on human IgG binding to SpA mutants the protocol described in Kim et al. 2010 was followed with few modifications.

In short, wells were coated with 200 ng immobilized SpA_WT-49-339 (SEQ ID NO: 15) per well, diluted in PBS, pH 7.6. IgY from egg yolk of chickens immunized with different SpA variants (Davids Biotechnologie GmbH) adjuvanted with AddaVax™ (InvivoGen) were diluted two-fold (from 1:10 to 1:320) and added to the plates. The plates were incubated for 1 hour at RT. Fifty nanograms (50 ng) per well of human IgG (Sigma-Aldrich 12511, lot #O48M4868V) was added and incubated for 1 hour at RT. Dilution of goat anti-human IgG antibody conjugated with alkaline phosphatase 1:10,000 (Sigma-Aldrich A1541, lot #029M4838V) was used as secondary antibody. As substrate, 3 mg/mL p-nitrophenyl phosphate (p-NPP) (Sigma-Aldrich P5869-25CAP, lot #SLBV2576) in diethanolamine (DEA, pH 9.2) (Sigma-Aldrich, D8885-500 g, lot #MKCB9120) was used. The absorbance at 495 nm was read after 55 min reaction using a Tecan Infinite M200Pro device. The PBS sample was set to 100% (control for no inhibition) and the percentage of human IgG binding was calculated using the formula:

$$( mean absorbance at 405 nm ) \div ( PBS absorbance at 405 nm ) \times 100 \%$$

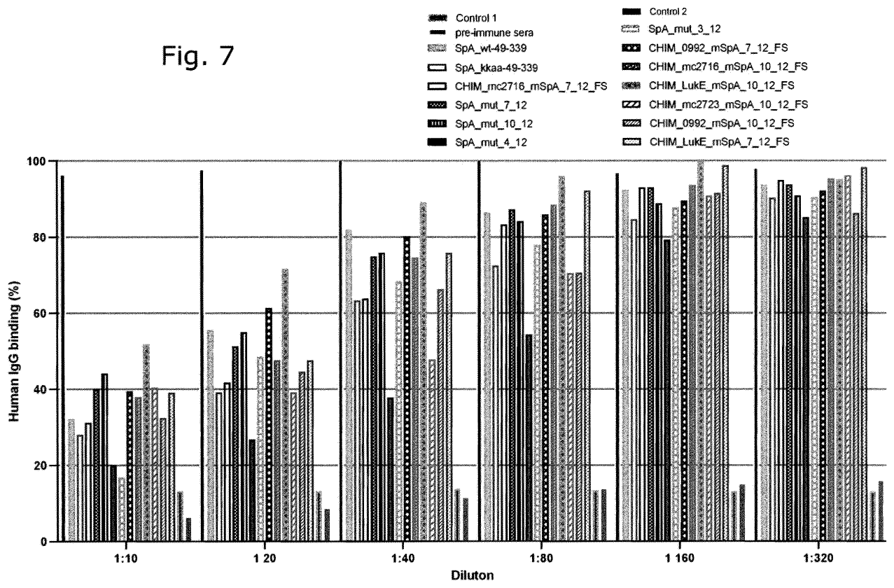

The results showed that the non-specific binding of human IgG could be outcompeted with specific IgY antibodies against several of the SpA mutant variants tested in a competitive ELISA (FIG. 7). Specific IgY against the chimeric protein, CHIM_mc2716_mSpA_7_12_FS (SEQ ID NO: 57), effectively blocked SpA binding of human IgG in a dose dependent manner (63% binding to human IgG at dilution 1:40, where the SpA_WT-49-339 had a human IgG binding at 82%). IgY specific to several SpA mutant variants; CHIM_mc2716_mSpA_7_12_FS (SEQ ID NO: 57), SpA_mut_7_12 (SEQ ID NO:18), SpA_mut_10_12 (SEQ ID NO: 17), SpA_mut_4_12 (SEQ ID NO: 19), SpA_mut_3_12 (SEQ ID NO: 20), CHIM_0992_mSpA_7_12_FS (SEQ ID NO: 58), CHIM_mc2716_mSpA_10_12_FS (SEQ ID NO: 65), CHIM_mc2723_mSpA_10_12_FS (SEQ ID NO: 83), CHIM_0992_mSpA_10_12_FS (SEQ ID NO: 84) and CHIM_LukE_mSpA_7_12_FS (SEQ ID NO: 85), all resulted in decreased human IgG binding to SpA_WT-49-339 (SEQ ID NO: 15) to different degrees. Pre-immune sera, collected before first immunization (negative control), did not have capacity to block unspecific binding of human IgG to SpA_WT-49-339. IgY specific for SpA_{KKAA}-49-339 (positive control) blocked SpA_WT-49-339 binding of human IgG.

### 1.2 Aureolysin Variants

An azocasein hydrolysis assay was set up to analyse the potential of vaccine candidate-specific antibodies to inhibit aureolysin enzymatic activity. Azocasein, a chromogenic derivate of casein, was used as a substrate to determine aureolysin activity. Aureolysin degrades azocasein to yield TCA-soluble (trichloracetic acid) azopeptides with high UV-absorbance which can be spectrophotometrically quantified. Therefore, aureolysin was incubated with azocasein and azocasein degradation was evaluated. Thermolysin, a thermostable neutral metalloproteinase enzyme produced by the Gram-positive bacteria Bacillus thermoproteolyticus (Sigma-Aldrich, P1512), was used as a positive control. Initially, commercial aureolysin and USA300HOU_2637 were tested for casein hydrolysis activity.

However, as neither of these aureolysins showed enzymatic activity in the assay (data not shown), a protocol for aureolysin purification from S. aureus USA300 culture was set up.

The protocol for aureolysin purification from S. aureus USA300 culture was as follows: 1 L S. aureus USA300HOU cell culture was grown in TSB (tryptic soy broth) medium for 24 hours at 37° C. and with 170 r.p.m agitation. The culture was harvested by centrifugation (4,100 r.p.m, 30 min at 4° C.) and the supernatant was filtrated using a 0.22 μm sterile filter. The supernatant was concentrated using Tangential Flow Filtration (TFF) (General Electric Healthcare, AKTA Flux) with a 10 kDa hollow fiber (General Electric Healthcare, AKTA Flux, UFP-10-C-3MA, batch 17000576). Precipitation of sterile supernatant was performed with 60% ammonium sulphate at 4° C. (according to Banbula et al., 1998). To stabilize pH, 1 M Tris (pH 8.0) was added at a ratio of 1:10 (v/v) before precipitation. The suspension was centrifugated at 10,000×g for 30 min at 4° C. Pellet was resuspended in 10 mM Tris-HCl, pH 7.6 and 5 mM CaCl₂. Dialysis was performed against 10 mM Tris-HCl pH 7.6 and 5 mM CaCl₂ overnight at 4° C. (10 kDa membrane cut-off). Hereafter two-steps chromatography was performed; (1) Akta purification: anion exchange—Mono Q 5/50 GL (General Electric Healthcare, 17-5166-01, lot #10244496)—elution with a gradient of 0-0.5 M NaCl, (2) Size Exclusion Chromatography (SEC): SUPERDEX 75 μg 16/600 (General Electric Healthcare, 28-9893-33, lot #10244501). The fractions were analysed by SDS-PAGE (using TGX stain free protein gel, Bio-Rad and using Pro Blue Safe Stain, Giotto Biotech) and Western Blot (primary antibody raised in rabbit 1 μg/mL; goat anti-rabbit IgG/HRP-conjugated (Dako, P0448, lot #20066477) diluted 1:2,000).

The purified aureolysin from S. aureus culture showed >90% purity (as estimated by SDS), and this purified aureolysin also showed activity in the casein hydrolysis assay. Mass spectrometry analyses confirmed the quality of aureolysin preparation (good peptide coverage of the mature form) (data not shown).

The protocol for the azocasein (activity) assay was as follows: For this assay 1 μg of thermolysin (from Geobacillus stearothermophilus, Sigma-Aldrich, P1512) was used as positive control. One microgram (1 μg) of thermolysin together with 10 mM EDTA was used as negative control (EDTA acts as inhibitor for metalloproteases). Using a solution of 10 mg/mL azocasein in McIlavaine buffer (pH 7.4, 10 mM CaCl₂ (19.07 mL 0.2 M Na₂HPO₄, 1.73 mL of 0.1 M citric acid, 0.2 mL of 1 M CaCl₂) was prepared. Mixtures of 200 μL of azocasein solution with PBS supplemented with 2 μg of purified aureolysin and either 20 μL or 50 μL of sera from mice immunized with different antigens, or with the adjuvant alone (negative control), were prepared. The mixtures were incubated at 37° C. with shaking (1,000 r.p.m) for 1 hour in a thermomixer at RT. The reaction was stopped by adding 400 μL 5% TCA (trichloracetic acid). Samples were centrifuged at 10,000×g for 3 min at 4° C. Next, 300 μL of the supernatant was transferred to a 96-multiwell plate, and the absorbance at 400 nm was read using a Tecan Infinite M200Pro device (Serial number: 1206001022).

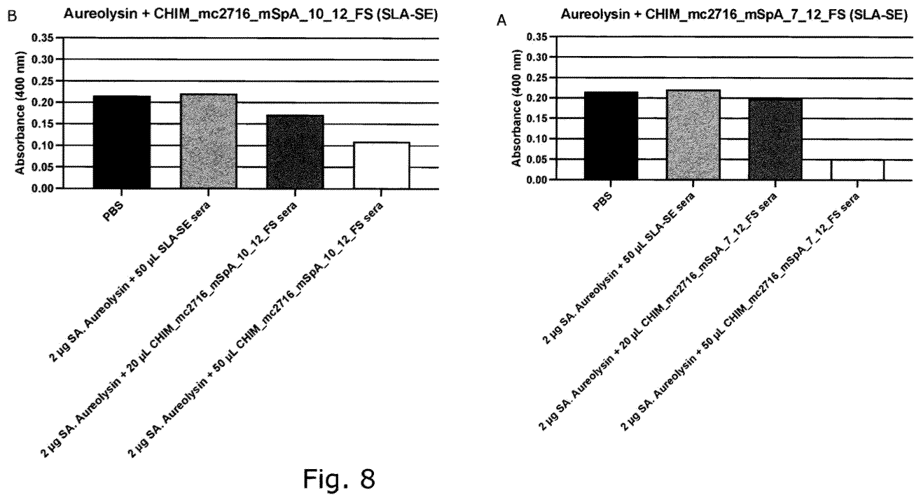

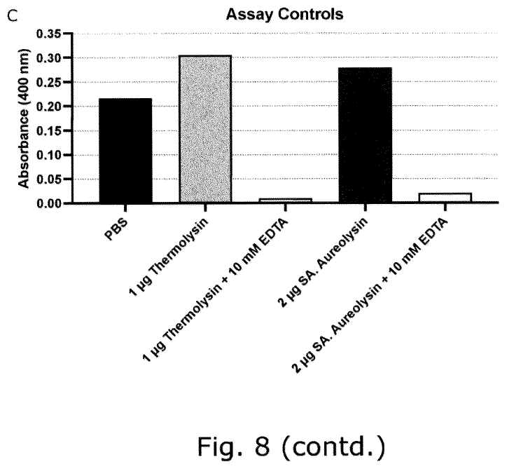

Activity of aureolysin purified from a S. aureus culture (named SA. Aureolysin) could be inhibited using immune sera from mice immunized with aureolysin chimeric protein constructs (FIG. 8). Immune sera from mice having received the CHIM_mc2716_mSpA_7_12_FS (SEQ ID NO: 57) adjuvanted with SLA-SE, showed strong capacity to inhibit enzymatic aureolysin activity in a dose dependent manner (FIG. 8A), as observed by a decrease in absorbance at 400 nm when compared to sera from mice immunized with PBS or SLA-SE adjuvant. This dose dependent aureolysin inhibition was also observed for immune sera collected from mice immunized with CHIM_mc2716_mSpA_10_12_FS (SEQ ID NO: 65) adjuvanted with SLA-SE (FIG. 8B). The assay controls confirm the assay setup (FIG. 8C). Several assays have been repeated with comparable outcomes.

### 1.3 LukE Variants

The LukE protein is one of two components of the LukED leukocidin. LukE and LukD subunits bind together to create a pore-forming toxin in the membrane of immune cells and erythrocytes resulting in lysis and killing. The target immune cells (in humans and mice) of LukED include neutrophils, monocytes, and macrophages amongst others. Therefore, LukE specific functional antibodies can inhibit the formation of the LukED toxin and the cytotoxic activity on human neutrophil cells.

The inhibition of the leukocytic activity of antibodies in sera from mice immunized with LukE or with chimeric protein including LukE, and adjuvanted with SLA-SE, was evaluated by a cytotoxin inhibition assay (XTT Assay). The human promyelocytic leukemia (HL-60) cell line was cultured as described by ATCC following the reported culture conditions. HL-60 cells were differentiated into neutrophil-like cells by the addition of 0.78% dimethylformamide (DMF) to the culture medium and incubation for 5 days at 37° C. with 5% CO₂. For the assay, 3.5-4×10⁵ cells per well were used. The assay was performed in a 96-well plate by adding to each well the components in the following order (duplicates were applied):
Tested sera diluted in culture medium in a final volume of 50 μL (two-fold dilutions from 1:50 to 1:400 dilution)
Toxin; LukE only, LukD only and LukE together with LukD, diluted in culture medium at the needed final concentration in a final volume of 40 μL.
3.5-4×10⁵ HL-60 differentiated cells in a final volume of 10 μL.

Following plating, the plate was incubated 24 hours at 37° C. with 5% CO₂. The cell viability was measured after 16 hours incubation with Cell proliferation kit II (XTT assay reagent, Sigma-Aldrich), following the manufacturer's protocol by reading absorbance at 470/690 nm. The colorimetric assay is based on the reduction of the yellow tetrazolium salt XTT (sodium 3′-[1-(phenylaminocarbonyl)-3,4-tetrazolium]-bis (4-methoxy6-nitro) benzene sulfonic acid hydrate) to an orange formazan dye by metabolically active cells. Finally, data were acquired with a SpectraMax Reader II Instrument.

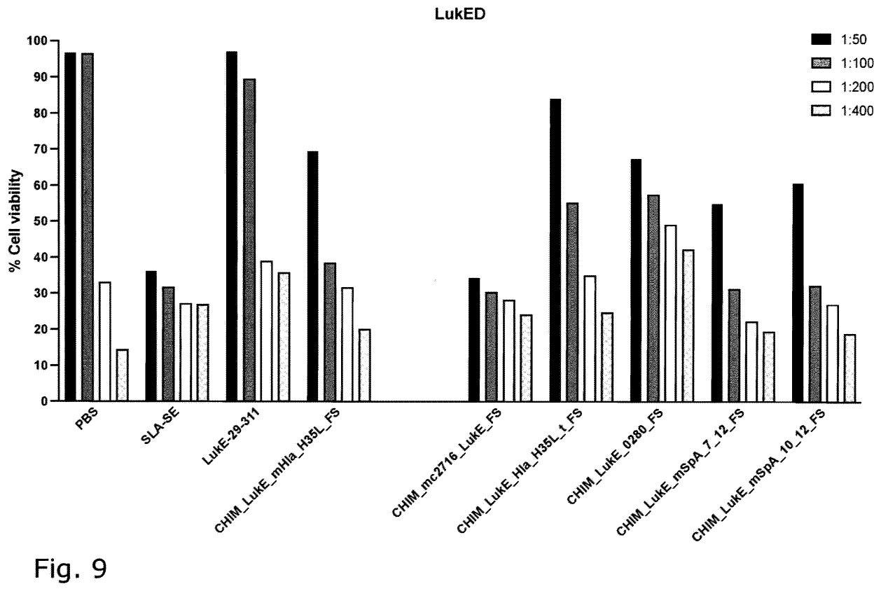

Immunization of mice with LukE-29-311 (SEQ ID NO: 30; positive control) adjuvanted with SLA-SE, or chimeric proteins including LukE adjuvanted with SLA-SE, elicited high amounts of functional antibodies capable of neutralizing the cytotoxic activity of LukED, compared to sera from mice immunized with the SLA-SE adjuvant only (FIG. 9).

The leukotoxicity of the functional wildtype LukE, can be blocked/neutralized by antibodies induced by the CHIM_LukE_mHla_H35L_FS (SEQ ID NO: 56) chimeric protein. Sera from mice immunized with several other LukE chimeric proteins adjuvanted with SLA-SE induce high cell viability, comparable to levels observed for the positive control, native LukE-29-311 immune sera (FIG. 9). The assay also showed that the toxin subunits LukE and LukD alone had no effect on cell viability across all tested chimeric proteins (data not shown).

### 1.4 Hla Variants

To evaluate the efficacy of different chimeric proteins containing designs of the Hla subunit, a haemolysis inhibition assay was set up. Mice were immunized two times subcutaneously (s.c.) into the flank using a two-week schedule, with different chimeric proteins adjuvanted with the SLA-SE. Immune sera were collected 14 days post 2^{nd} immunization to assess induction of specific antibodies. The immune sera were two-fold serially diluted (from 1:10 to 1:640) in PBS with 0.5% bovine serum albumin (BSA) in a 96-well plate. Each serum dilution was incubated with 20 ng recombinant Hla (rHla) for 20 min at RT. Water only and a solution of PBS with 0.5% BSA were used as positive and negative controls, respectively. Two percent (2%) solution of rabbit erythrocytes derived from defibrinated rabbit blood was added to the plate and the plate incubated at 37° C. for 30 min. After centrifugation at 1,000×g at 4° C. for 5 min, supernatants were transferred into a new 96-well plate. One-hundred microliters (100 μL) PBS was added to each well and absorbance at 540 nm was read with a plate reader. Percentage of haemolysis was calculated with respect to the haemolysis induced by water (100% haemolysis).

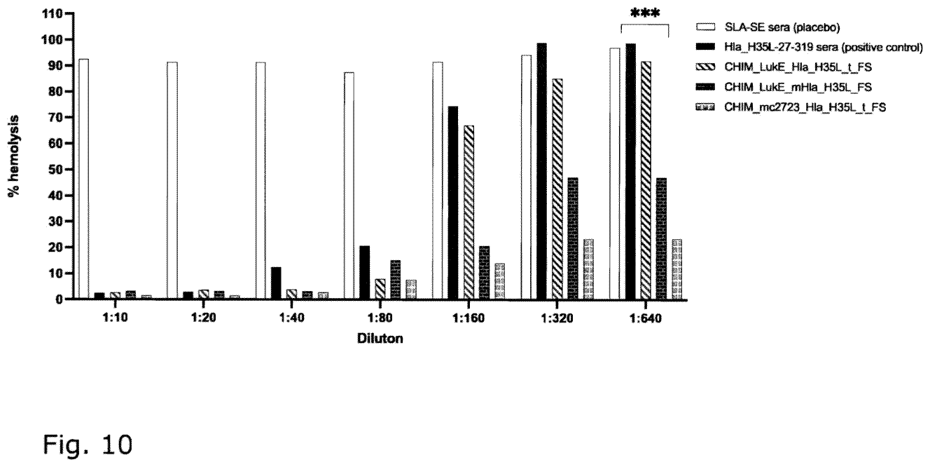

Sera from mice immunized with adjuvant alone (SLA-SE) did not inhibit haemolysis of erythrocytes (FIG. 10). The positive control sera collected from mice having received Hla_H35L-27-319 (SEQ ID NO: 5) with SLA-SE, showed inhibition of haemolysis, which was lost at dilution 1:320. Both control sera together verified the setup of the haemolysis inhibition assay. Immune sera from mice immunized with the chimeric protein CHIM_LukE_mHla_H35L_FS (SEQ ID NO: 56) together with SLA-SE, showed strong capacity to inhibit haemolysis even at highest dilution tested (1:640). This chimeric protein immune sera inhibited 53% haemolysis at dilution 1:640, whereas both sera from mice having received placebo (SLA-SE) and positive control (Hla_H35L-27-319), only inhibited approx. 1-3% haemolysis at the same dilution. Different chimeric proteins all containing the Hla subunit, were capable of inducing haemolysis inhibitory immune sera to various degrees. However, highest haemolysis inhibition effect was observed with immune sera from mice having received CHIM_mc2723_Hla_H35L_t_FS (SEQ ID NO: 62), showing 77% haemolysis inhibition at dilution 1:640. The assay was repeated with comparable results.

### Example 2: Prophylactic Vaccine Efficacy in Animal Challenge Model

Efficacy of the prophylactic vaccine proteins was evaluated using a peritonitis animal model of S. aureus infection using CD-1 mice (6-8-week-old females, 6-12 mice per group, Charles River, Germany). Prior to challenge, mice were immunized twice or three times either intramuscular (i.m.) into their hind legs (split) at day 0 and day 14 (for two immunizations) or at day 0, day 14 and day 28 (for three immunizations) or subcutaneously (s.c.) into the flank with prophylactic vaccine products stated in Table 5 adjuvanted with either outer membrane vesicle (OMV, InvivoGen, E. coli OMV InvivoFit™) with aluminum hydroxide gel (AIOH, Alhydrogel, InvivoGen, Cat. Code: vac-alu-250, Lot #5531), aluminum hydroxide gel (AIOH, Alhydrogel, InvivoGen, Cat. Code: vac-alu-250, Lot #5531) alone or SLA-SE (AAHI, the Access to Advanced Health Institute). The proteins were formulated 1:1[vol/vol] with the above stated adjuvants diluted in a 10 mM Tris+144 mM NaCl, pH 7.2 buffer. The dosage of the single proteins or chimeric constructs are stated in Table 5. As a negative control, mice were immunized with the adjuvant alone or with saline.

The OMV+AIOH adjuvant was prepared as follows: 500 μg OMVs from InvivoGen was resuspended in 1 mL of the supplied sterile, endotoxin free water, and aliquoted into 200 μL aliquots. For a group of 8 mice, 96 μL of the OMV-suspension was mixed with 144 μL sterile buffer. Then 240 μL of redispersed aluminum hydroxide gel was added to the OMV/buffer solution for a final concentration of 5 μL OMV/50 μL, rigorously mixed, and kept on ice in the fridge for at least one hour prior to immunization.

Two or four weeks post last immunization, mice were challenged by intraperitoneal (i.p.) injection with either the S. aureus USA300 LAC strain (NCBI: CP000730.1, Diep et al., 2006), the S. aureus MRSA252 strain (NCBI: BX571856.1), the S. aureus Newman strain (NCBI: AP009351.1) or the S. aureus USA400 (NCBI:NZ_CP019574.1) with different challenge CFU dosages as stated in Table 5.

To generate challenge doses of different S. aureus strains protocol was as follows: Overnight cultures were diluted to an optical density of 600 nm (OD_{600 nm}) of 0.5 into fresh tryptic soy broth (TSB) and grown for approx. 2.5 hours at 37° C. until an OD_{600 nm} of 1.2 was achieved. Bacteria were then harvested by centrifugation, washed twice, and concentrated in 2% of PBS+16% glycerol. Culture was aliquoted and immediately stored at −80° C. for later use. Final challenge dose administered to the animals was verified experimentally by plating culture on agar plates and counting CFU the following day.

Animals were monitored for 7 days post bacterial challenge, studying predefined clinical signs of infection, and using humane endpoint criteria to determine if mice should be sacrificed. The overall survival was recorded.

The survival of the challenged mice showed that immunizations with EDEN Combo-1 consisting of CHIM_mc2716_mSpA_7_12_FS (SEQ ID NO: 57), CHIM_2753_mc2723_FS (SEQ ID NO: 59), SAR0280-28-820 (SEQ ID NO: 48) and SAR0992-1-409 with or without the toxoid CHIM_LukE_mHla_H35L_FS (SEQ ID NO: 56), adjuvanted with OMV+AIOH resulted in highly significantly protection (87.5% survival, p=<0.0001) when immunized three times i.m. as shown in Table 5 compared to the SHAM saline control.

When immunized three times i.m. with EDEN Combo-1 adjuvanted with OMV+AIOH without the toxoid chimeric protein, 50% survival towards the USA300 LAC strain was achieved (p=0.0013, **) compared to the SHAM saline control. It was also observed that EDEN Combo-1 with the toxoid chimeric protein adjuvanted with AIOH induced high protection against the USA300 LAC strain (p=<0.0001, ****). In this study, immunization with EDEN Combo-1 antigens with or without toxoid chimeric protein resulted in strong protection, regardless of the adjuvant. For statistical evaluation Log-Rank Mantel-Cox test was employed.

After challenge with USA300 LAC or MRSA252, LukE-29-311 (SEQ ID NO: 30) SLA-SE adjuvanted vaccines resulted in significant protection. In addition, vaccinations with either USA300HOU_2637_E353A-28-509 (SEQ ID NO: 35), SAR2723-28-509 (SEQ ID NO: 39), or SAR0280-28-820 (SEQ ID NO: 48) adjuvanted with SLA-SE resulted in significant protection against challenge with USA300 LAC. Immunization with either SAR2753-291-680 (SEQ ID NO: 42) or some of the chimeric protein constructs in Table 5 adjuvanted with SLA-SE, all resulted in protection against challenge with MRSA252. Immunization with SAR0992-1-409 adjuvanted with SLA-SE resulted in significant protection against challenge with USA300 LAC, USA400 and Newman strains. For statistical evaluation Log-Rank Mantel-Cox test was employed.

TABLE 5
Sepsis challenge model protection data.

| | | | | | Challenge | |
|---|---|---|---|---|---|---|
| | | Antigen | Immunization | | interval post | |
| | | Dose | route | No. of | immunization | Bacterial |
| Antigen | Adjuvant | (μg)* | (i.m. or s.c.) | immunizations | (weeks) | strain |
| CHIM_mc2716_mSpA_7_12_FS + | OMV + | 60 | i.m. | 3 | 4 | USA300 LAC |
| CHIM_2753_mc2723_FS + | AIOH | | | | | |
| SAR0280-28-820 + SAR0992-1-409 | | | | | | |
| CHIM_mc2716_mSpA_7_12_FS + | OMV + | 80 | i.m. | 3 | 4 | USA300 LAC |
| CHIM_2753_mc2723_FS + | AIOH | | | | | |
| SAR0280-28-820 + SAR0992-1-409 + | | | | | | |
| CHIM_LukE_mHla_H35L_FS | | | | | | |
| CHIM_mc2716_mSpA_7_12_FS + | AIOH | 80 | i.m. | 3 | 4 | USA300 LAC |
| CHIM_2753_mc2723_FS + | | | | | | |
| SAR0280-28-820 + SAR0992-1-409 + | | | | | | |
| CHIM LukE mHla H35L FS | | | | | | |
| CHIM_0280_2753_FS | SLA-SE | 30 | s.c. | 3 | 2 | MRSA252 |
| CHIM_0992_2753_FS | SLA-SE | 50 | s.c. | 3 | 2 | MRSA252 |
| CHIM_2753_mc2723_FS | SLA-SE | 20 | s.c. | 2 | 2 | Newman |
| CHIM_LukE_0280_FS | SLA-SE | 30 | s.c. | 3 | 3 | MRSA252 |
| CHIM_0992_m0992_FS | SLA-SE | 30 | s.c. | 3 | 2 | MRSA252 |
| CHIM_mc2716_LukE_FS | SLA-SE | 30 | s.c. | 3 | 3 | MRSA252 |
| LukE-29-311 | SLA-SE | 30 | i.m. | 2 | 2 | MRSA252 |
| LukE-29-311 | SLA-SE | 10 | s.c. | 3 | 2 | USA300 LAC |
| SAR0280-28-820 | SLA-SE | 10 | s.c. | 3 | 2 | USA300 LAC |
| SAR2723-28-619 | SLA-SE | 10 | s.c. | 3 | 2 | USA300 LAC |
| SAR2753-291-680 | SLA-SE | 30 | i.m. | 3 | 2 | MRSA252 |
| USA300HOU 2637 E353A-28-509 | SLA-SE | 10 | s.c. | 3 | 2 | USA300 LAC |
| CHIM_LukE_Hla_H35L_t_FS | SLA-SE | 30 | s.c. | 3 | 3 | MRSA252 |
| Hla_H35L-39-319 | SLA-SE | 20 | s.c. | 2 | 2 | Newman |
| SAR0992-1-409 | SLA-SE | 10 | s.c. | 3 | 2 | USA300 LAC |
| SAR0992-1-409 | SLA-SE | 10 | s.c. | 2 | 2 | Newman |
| SAR0992-1-409 | SLA-SE | 15 | s.c. | 3 | 2 | USA400 |
| | | | | | | (M3496) |

| | | % Immunized | % Negative | Significance |
|---|---|---|---|---|
| | Challenge | mice | control mice | (log-rank, |
| | dose | survival post | survival post | Mantel- |
| Antigen | (CFU/mouse | challenge | challenge | Cox, test) |
| CHIM_mc2716_mSpA_7_12_FS + | 5 × 10⁸ | 50 | 8.33 | ** |
| CHIM_2753_mc2723_FS + | | | | |
| SAR0280-28-820 + SAR0992-1-409 | | | | |
| CHIM_mc2716_mSpA_7_12_FS + | 5 × 10⁸ | 87.5 | 8.33 | **** |
| CHIM_2753_mc2723_FS + | | | | |
| SAR0280-28-820 + SAR0992-1-409 + | | | | |
| CHIM_LukE_mHla_H35L_FS | | | | |
| CHIM_mc2716_mSpA_7_12_FS + | 5 × 10⁸ | 75 | 8.33 | **** |
| CHIM_2753_mc2723_FS + | | | | |
| SAR0280-28-820 + SAR0992-1-409 + | | | | |
| CHIM LukE mHla H35L FS | | | | |
| CHIM_0280_2753_FS | 2 × 10⁸ | 50 | 25 | * |
| CHIM_0992_2753_FS | 2 × 10⁸ | 58 | 25 | * |
| CHIM_2753_mc2723_FS | 4.1 × 10⁸ | 25 | 0 | ns |
| CHIM_LukE_0280_FS | 2 × 10⁹ | 58 | 25 | ns |
| CHIM_0992_m0992_FS | 2 × 10⁹ | 50 | 27 | ns |
| CHIM_mc2716_LukE_FS | 2 × 10⁹ | 50 | 25 | ns |
| LukE-29-311 | 1.5 × 10⁹ | 42 | 0 | * |
| LukE-29-311 | 2.7 × 10⁸ | 63 | 0 | ** |
| SAR0280-28-820 | 2.7 × 10⁸ | 50 | 0 | ** |
| SAR2723-28-619 | 2.7 × 10⁸ | 50 | 0 | ** |
| SAR2753-291-680 | 1.5 × 10⁹ | 50 | 0 | ** |
| USA300HOU 2637 E353A-28-509 | 2.7 × 10⁸ | 63 | 0 | ** |
| CHIM_LukE_Hla_H3SL_t_FS | 2 × 10⁹ | 92 | 25 | *** |
| Hla_H35L-39-319 | 3 × 10⁸ | 88 | 0 | *** |
| SAR0992-1-409 | 2.7 × 10⁸ | 88 | 0 | *** |
| SAR0992-1-409 | 4.1 × 10⁸ | 75 | 18 | * |
| SAR0992-1-409 | 1 × 10⁸ | 50 | 6 | * |

*In the case of combinations, the antigen dose shown is the total dose given. In the combinations adjuvanted with OMV + AIOH or AIOH alone, each chimeric protein was given at a dose of 20 μg, and each single antigen was given in a dose of 10 μg.

### Example 3: Prophylactic Vaccine Efficacy in Skin Model

Efficacy of the prophylactic vaccine proteins was evaluated using a skin abscess animal model of S. aureus infection using BALB/c mice (6-weeks old females, 12 mice per group). Prior to challenge, mice were immunized twice or three times, either intramuscular (i.m) into their hind legs (split) at day 0 and day 14 (for two immunizations) or at day 0, day 14 and day 28 (for three immunizations) or subcutaneous (s.c.) into the flank with the prophylactic vaccine product as stated in Table 6 adjuvanted with either aluminum hydroxide (AIOH, Alhydrogel, InvivoGen, Cat. Code: vac-alu-250, Lot #5531) or SLA-SE (AAHI, the Access to Advanced Health Institute). The proteins were formulated 1:1[vol/vol] with the above stated adjuvants diluted in a 10 mM Tris+144 mM NaCl, pH 7.2 buffer. The dosage of the single proteins or chimeric constructs is stated in Table 6. As a negative control, mice were immunized with the adjuvant alone or with saline.

Two weeks post last immunization, mice were challenged by subcutaneous (s.c.) injection with either the S. aureus USA300 LAC strain (Diep et al., 2006) or the S. aureus Newman strain (Baba et al., 2008) with different challenge CFU dosages as stated in Table 6.

To generate challenge doses of different S. aureus strains protocol was as follows: Overnight cultures were diluted to an optical density of 600 nm (OD_{600 nm}) of 0.5 into fresh tryptic soy broth (TSB) and grown for approx. 2.5 hours at 37° C. until an OD_{600 nm} of 1.2 was achieved. Bacteria were then harvested by centrifugation, washed twice, and concentrated in 2% of PBS+16% glycerol. Culture was aliquoted and immediately stored at −80° C. for later use. Final challenge dose administered to the animals was verified experimentally by plating culture on agar plates and counting CFU the following day.

Abscess formation (size and dermonecrotic lesions) was monitored at 24-hour intervals over a course of 10 days post infection. The size of an abscess and associated overlying dermonecrotic lesions was determined using a standard formula for ellipse area: [A=n×lesion length/2×lesion width/2]. For statistical evaluation of the area under the curve (AUC), Two-way ANOVA (group×time) on skin lesion area parameter was performed, followed with Dunnett's multiple comparison test (Kruskal-Wallis test). Difference between groups were considered statistically significant when p-values <0.05. The statistical analysis and AUC data were analyzed using GraphPad Prism version 9. High significance difference was considered when the p-value<0.0001 and indicated with ‘****’. The ‘ns’ (non-significant) refer to p-values>0.05.

As a result of immunization with the EDEN Combo-1 consisting of a combination of CHIM_mc2716_mSpA_7_12_FS (SEQ ID NO: 57), CHIM_2753_mc2723_FS (SEQ ID NO: 59), SAR0280-28-820 (SEQ ID NO: 48) and SAR0992-1-409 adjuvanted with AIOH, significantly impaired USA300 LAC abscess formation, thus resulting in significant protection (p=0.0114 *) compared to the AIOH adjuvant alone.

Immunization with either Hla_H35L-39-319 (SEQ ID NO: 6), LukE-29-311 (SEQ ID NO: 30), or SAR2753-291-680 (SEQ ID NO: 42) adjuvanted with SLA-SE highly significantly impaired abscess formations post USA300 LAC and Newman challenge compared to immunization with the SLA-SE adjuvant alone. Immunization with USA300HOU_2637_E353A-28-509 (SEQ ID NO: 35) adjuvanted with SLA-SE significantly impaired (*) abscess formation post USA300 LAC challenge. Immunization with either SAR2723-28-619 (SEQ ID NO: 39) or SAR0992-1-409 adjuvanted with SLA-SE significantly impaired abscess formation post Newman challenge. Immunization with different chimeric constructs adjuvanted with SLA-SE also resulted in lower AUC and impaired abscess formation when challenged with either the S. aureus USA300 LAC or S. aureus Newman strain. All groups were tested against SLA-SE adjuvanted alone.

TABLE 6
S. aureus skin abscess data.

| | | | | | Challenge |
|---|---|---|---|---|---|
| | | Antigen | Immunization | | interval post |
| | | Dose | route | No. of | immunization |
| Antigen | Adjuvant | (μg)* | (i.m. or s.c.) | immunizations | (weeks) |
| EDEN Combo-1 | AIOH | 60 | i.m. | 3 | 2 |
| (CHIM_mc2716_mSpA_7_12_FS, | | | | | |
| CHIM_2753_mc2723_FS, | | | | | |
| SAR0280_28-820 and SAR0992-1- | | | | | |
| 409) | | | | | |
| CHIM_LukE_0280_FS | SLA-SE | 40 | s.c. | 3 | 2 |
| CHIM_m0992_0992_FL | SLA-SE | 40 | i.m. | 3 | 2 |
| CHIM_mc2716_LukE | SLA-SE | 40 | s.c. | 2 | 2 |
| LukE-29-311 | SLA-SE | 20 | s.c. | 2 | 2 |
| LukE-29-311 | SLA-SE | 20 | i.m. | 3 | 2 |
| LukE-29-311 + SAR0992-1-409 + | SLA-SE | 80 | i.m. | 3 | 2 |
| SAR0280-28-820 + SAR2723-28- | | | | | |
| 619 | | | | | |
| SAR2723-28-619 | SLA-SE | 20 | s.c. | 2 | 2 |
| SAR2753-291-680 | SLA-SE | 20 | s.c. | 2 | 2 |
| SAR2753-291-680 | SLA-SE | 20 | i.m. | 3 | 2 |
| USA300HOU_2637_E353A-28- | SLA-SE | 20 | i.m. | 3 | 2 |
| 509 | | | | | |
| CHIM_LukE_Hla_H35L_t_FS | SLA-SE | 40 | s.c. | 3 | 2 |
| CHIM_LukE_Hla_H35L_t_FS | SLA-SE | 40 | s.c. | 2 | 2 |
| Hla_H35L_39-319 | SLA-SE | 20 | i.m. | 3 | 2 |
| Hla_H35L_39-319 | SLA-SE | 20 | s.c. | 2 | 2 |
| SAR0992-1-409 | SLA-SE | 20 | s.c. | 2 | 2 |

| | | Challenge | | | Significance |
|---|---|---|---|---|---|
| | | dose | AUC of | | level |
| | Bacterial | (CFU/100 | test | AUC of | (Kruskal- |
| Antigen | strain | μl/mouse) | antigen | placebo | Walli's test) |
| EDEN Combo-1 | USA300 LAC | 5 × 10⁸ | 1230 | 1633 | * |
| (CHIM_mc2716_mSpA_7_12_FS, | | | | | |
| CHIM_2753_mc2723_FS, | | | | | |
| SAR0280_28-820 and SAR0992-1- | | | | | |
| 409) | | | | | |
| CHIM_LukE_0280_FS | USA300 LAC | 9.3 × 10⁸ | 784 | 1019 | ** |
| CHIM_m0992_0992_FL | USA300 LAC | 1 × 10⁹ | 1096 | 1513 | ** |
| CHIM_mc2716_LukE | Newman | 1 × 10⁸ | 577 | 1006 | ** |
| LukE-29-311 | Newman | 1 × 10⁸ | 685 | 1429 | ** |
| LukE-29-311 | USA300 LAC | 5 × 10⁸ | 776 | 853 | * |
| LukE-29-311 + SAR0992-1-409 + | USA300 LAC | 5 × 10⁸ | 742 | 853 | ns |
| SAR0280-28-820 + SAR2723-28- | | | | | |
| 619 | | | | | |
| SAR2723-28-619 | Newman | 1 × 10⁸ | 646 | 1153 | ** |
| SAR2753-291-680 | Newman | 1 × 10⁸ | 841 | 1153 | * |
| SAR2753-291-680 | USA300 LAC | 8.9 × 10⁸ | 1054 | 1504 | * |
| USA300HOU_2637_E353A-28- | USA300 LAC | 8.9 × 10⁸ | 1148 | 1504 | * |
| 509 | | | | | |
| CHIM_LukE_Hla_H35L_t_FS | USA300 LAC | 1.19 × 10⁹ | 421 | 1281 | **** |
| CHIM_LukE_Hla_H35L_t_FS | Newman | 1 × 10⁸ | 320 | 1006 | *** |
| Hla_H35L_39-319 | USA300 LAC | 8.9 × 10⁸ | 675 | 1504 | **** |
| Hla_H35L_39-319 | Newman | 1 × 10⁸ | 263 | 1153 | *** |
| SAR0992-1-409 | Newman | 1 × 10⁸ | 902 | 1153 | * |

*In the case of combinations, the antigen dose shown is the total dose given. In the combinations adjuvanted with AIOH, each chimeric protein was given at a dose of 20 μg, and each single antigen was given in a dose of 10 μg. In combination adjuvanted with SLA-SE, each single antigen was given in a dose of 20 μg.

### Example 4: Prophylactic Vaccine Immunogenicity Igg Data

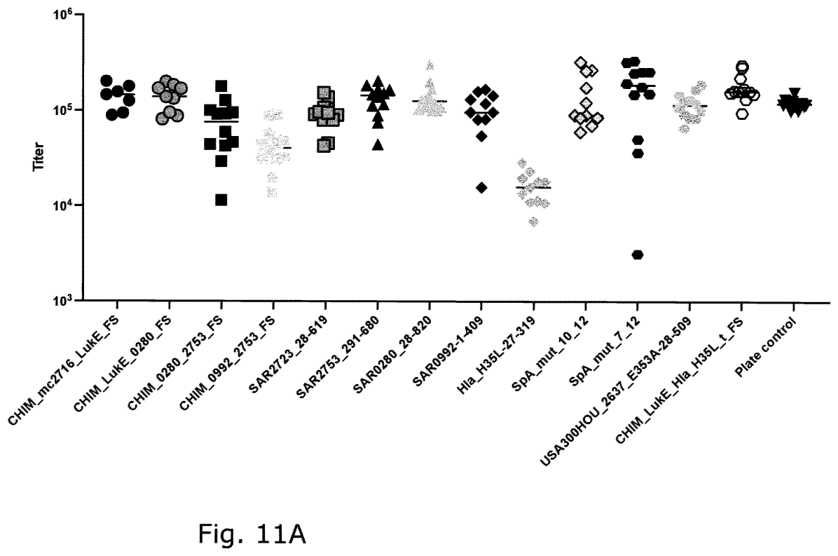

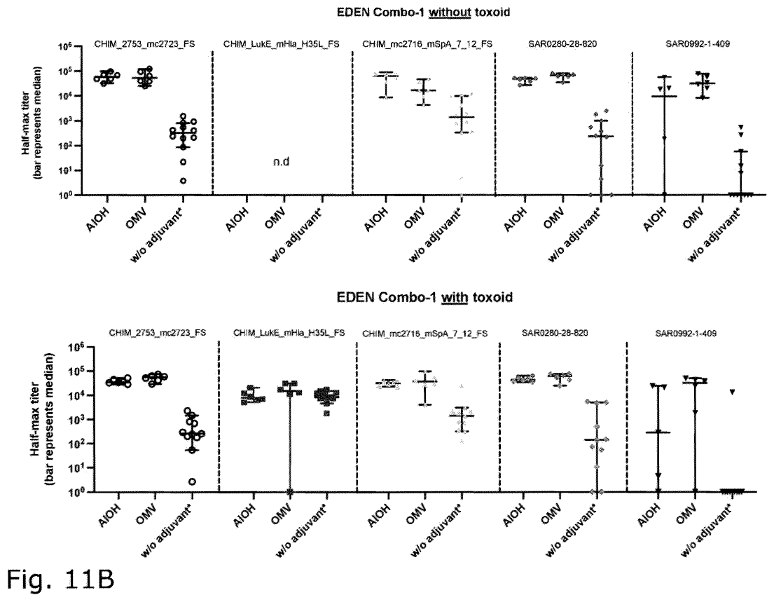

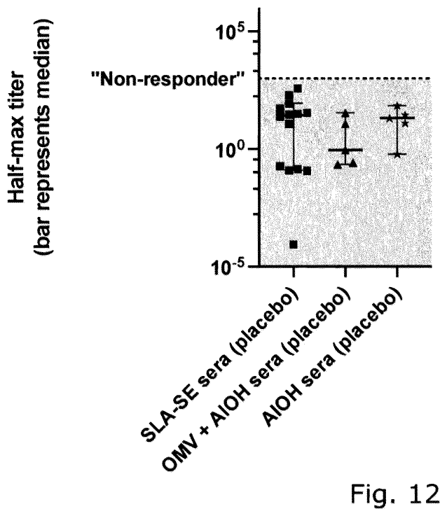

The total immunoglobulin G (IgG) response post immunization with the prophylactic vaccine proteins was evaluated using a standard ELISA assay. Mice were immunized three times with 30 μg per single protein or chimeric constructs for formulated with the adjuvant SLA-SE as stated in FIG. 11a or immunized with 20 μg per chimeric construct and 10 μg of single proteins in the EDEN Combo-1 consisting of CHIM_mc2716_mSpA_7_12_FS (SEQ ID NO: 57), CHIM_2753_mc2723_FS (SEQ ID NO: 59), SAR0280-28-820 (SEQ ID NO: 48) and SAR0992-1-409 with or without the toxoid chimeric construct CHIM_LukE_mHla_H35L_FS (SEQ ID NO: 56) added, formulated with either OMV (InvivoGen, E. coli OMV InvivoFit™)+AIOH or AIOH (InvivoGen) only as the adjuvant, as stated in FIG. 11b. Adjuvants were diluted in a 10 mM Tris+144 mM NaCl, pH 7.2 buffer. Immunizations were administered either intramuscularly (i.m) into the hind legs at day 0, day 14 and at day 28, or subcutaneously (s.c.) into the flank. Blood samples were drawn from the tail vein 12 days post immunization to assess antigen-specific total IgG titers.

A standard ELISA assay protocol was followed: The 96-well Nunc-Immuno™ MaxiSorp plate (Sigma-Aldrich) was coated with single or chimeric proteins and IgG half-max titers in sera from mice immunized with the respective proteins were determined. A threshold for assay sensitivity was set to half-max titer of 103. Each dot in FIG. 11a-c represents one immunized mouse. A plate control was included in each ELISA run to verify assay efficiency.

As shown in FIG. 11a, all tested immune sera from mice immunized with the prophylactic vaccines containing CHIM_mc2716_LukE_FS (SEQ ID NO:63), CHIM_LukE_Hla_H35L_t_FS (SEQ ID NO: 64), CHIM_LukE_0280_FS (SEQ ID NO: 66), CHIM_0992_2753_FS (SEQ ID NO: 61), SAR2723-28-619 (SEQ ID NO: 39), SAR2753-291-680 (SEQ ID NO: 42), SAR0280-28-820 (SEQ ID NO: 48), SAR0992-1-409, Hla_H35L-27-319 (SEQ ID NO: 5), SpA_mut_10_12 (SEQ ID NO: 17), SpA_mut_7_12 (SEQ ID NO:18), or USA300HOU_2637_E353A-28-509 (SEQ ID NO: 35) adjuvanted with SLA-SE, had high levels of antigen-specific total IgG antibodies compared to SLA-SA adjuvant alone (FIG. 11c).

As shown in FIG. 11b, the immune sera from mice immunized with the EDEN Combo-1 adjuvanted with either OMV+AIOH or AIOH alone induced high levels of antigen-specific total IgG antibodies against the single and chimeric proteins included in the EDEN Combo-1 compared to both the OMV+AIOH and AIOH adjuvants alone (FIG. 11c).

Data was analysed using GraphPad Prism version 9.0 by calculating the EC50 of each serum dilution curve using the Hill equation to model a non-linear fit and group means of the IgG titers for each immunization were calculated using Row Statistics computing the mean for each data set column. For statistical evaluation One-way ANOVA test, Tukey's multiple comparison test was employed. Significant difference was considered when the p-value≤0.05. High significance difference was considered when the p-value<0.0001 and indicated with ‘****’. The ‘ns’ (non-significant) refers to p-values>0.05. For the IgG subclasses, one sample t and Wilcoxon test was applied.

### REFERENCE LIST TO THE EXAMPLES

Ashley M. Vaughan (ed.), Malaria Vaccines: Methods and Protocols, Methods in Molecular Biology, vol. 1325, DOI 10.1007/978-1-4939-2815-6_16, Springer Science+Business Media New York 2015
Baba T, Bae T, Schneewind O, Takeuchi F, Hiramatsu K. J Bacteriol. 2008 January; 190(1):300-10. doi: 10.1128/JB.01000-07. Epub 2007 Oct. 19. PMID: 17951380; PMCID: PMC2223734.
Banbula A, Potempa J, Travis J, Fernandez-Catalen C, Mann K, Huber R, Bode W, Medrano F. Structure. 1998 Sep. 15; 6(9):1185-93. doi: 10.1016/s0969-2126(98)00118-x. PMID: 9753696.
Diep B A, Gill S R, Chang R F, Phan T H, Chen J H, Davidson M G, Lin F, Lin J, Carleton H A, Mongodin E F, Sensabaugh G F, Perdreau-Remington F. Lancet. 2006 Mar. 4; 367(9512):731-9. doi: 10.1016/50140-6736(06)68231-7. PMID: 16517273.
Kim H K et al., 2010 Nontoxigenic protein A vaccine for methicillin-resistant Staphylococcus aureus infections in mice. J Exp Med 207:1863-1870.
Kim H K et al., 2015 Protein A suppresses immune responses during Staphylococcus aureus bloodstream infection in guinea pigs. mBio 6.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. A vaccine composition comprising a selection of polypeptides, which includes:
a) an Alpha-hemolysin (Hla) polypeptide or a variant thereof, where said variant preferably exhibits reduced or no hemolytic activity and/or which preferably can induce antibodies that block the hemolytic activity of native Hla, wherein said polypeptide is an immunogenic polypeptide consisting of or comprising a variant of the amino acid sequence of Hla, which variant:
i. has at least 85% sequence identity with the sequence of SEQ ID NO: 2, and
ii. comprises one or more amino acid deletion(s) and/or substitution(s) in the first 12 consecutive N-terminal amino acid residues of the amino acid sequence of mature Hla, said first 12 consecutive N-terminal amino acid residues corresponding to positions 1 to 12 of SEQ ID NO: 2,
wherein the polypeptide is unable to participate in formation of a heptameric structure with other Hla molecules and wherein any substitution(s) in ii) are preferably non-conservative, and at least one of b-d:
b) a LukE polypeptide or a variant thereof, where said variant preferably exhibits reduced or no leukocytic activity and/or which preferably can induce antibodies that block the leukocytic activity of native LukE,
c) an Immunoglobulin G-binding protein A (SpA) polypeptide or a variant thereof, where said variant preferably exhibits a reduced ability to bind human IgG and human von Willebrandt Factor and/or which preferably can induce antibodies that block native SpA interaction with human IgG, and
d) an Aureolysin (Aur) polypeptide or a variant thereof, where said variant preferably exhibits reduced catalytic activity and which preferably can induce antibodies that block the catalytic activity of native Aur,
said composition optionally further comprising a pharmaceutically acceptable carrier, vehicle or diluent, and further optionally an immunogenic adjuvant, which is in particular selected from AIOH, SLA-SE and OMVs (outer membrane vesicles).

95. The vaccine composition according to claim 94, wherein the variant of the amino acid sequence of Hla comprises at least two amino acid alterations selected from deletions and substitutions in said first 12 consecutive N-terminal amino acid residues, such as at least or exactly 2, at least or exactly 3, at least or exactly 4, at least or exactly 5, at least or exactly 6, at least or exactly 7, at least or exactly 8, at least or exactly 9, at least or exactly 10, at least or exactly 11, or exactly 12 amino acid alterations.

96. The vaccine composition according to claim 94 or 95, wherein the variant of the amino acid sequence of Hla comprises at least two amino acid substitutions in the first 12 N-terminal amino acids under ii), such as at least or exactly 2, at least or exactly 3, at least or exactly 4, at least or exactly 5, at least or exactly 6, at least or exactly 7, at least or exactly 8, at least or exactly 9, at least or exactly 10, at least or exactly 11, or exactly 12 amino acid substitutions.

97. The vaccine composition according to claim 94 or 95, wherein the variant of the amino acid sequence of Hla comprises at least two amino acid deletions in said first 12 consecutive N-terminal amino acid residues, such as at least or exactly 2, at least or exactly 3, at least or exactly 4, at least or exactly 5, at least or exactly 6, at least or exactly 7, at least or exactly 8, at least or exactly 9, at least or exactly 10, at least or exactly 11, or exactly 12 amino acid deletions.

98. The vaccine composition according to claim 94, wherein amino acid residues 1-12 of the variant of the amino acid sequence of Hla are all deleted.

99. The vaccine composition according to claim 94, wherein amino acid residues 1-12 of the variant of the amino acid sequence of Hla are all substituted.

100. The vaccine composition according to claim 99, wherein amino acid residues 1-12 are substituted with the sequence 5′-SETEVSVRSASS-3′ (residues 1-12 in SEQ ID NO: 4).

101. The vaccine composition according to claim 94, wherein the variant of the amino acid sequence of Hla comprises SEQ ID NO: 3.

102. The vaccine composition according to claim 94, wherein the variant of the amino acid sequence of Hla comprises a substitution from histidine (H) to leucine (L) at the position corresponding to position 35 of SEQ ID NO: 2.

103. The vaccine composition according to claim 102, wherein the variant of the amino acid sequence of Hla comprises SEQ ID NO: 6.

104. The vaccine composition according to claim 94, wherein the variant of the amino acid sequence of Hla lacks or has significantly reduced hemolytic activity.

105. The vaccine composition according to claim 94, wherein the polypeptide consisting of or comprising a variant of the amino acid sequence of Hla can induce antibodies that block the hemolytic activity of wildtype Hla.

106. The vaccine composition according to claim 94, comprising the variant of the SpA polypeptide according to claim 94 c, which variant comprises:
i. at least one of a-e:
a. a sequence at least 85% identical to the amino acid sequence of Immunoglobulin binding domain (IgBD) E (SEQ ID NO: 16, residues 1-56),
b. a sequence at least 85% identical to the amino acid sequence of IgBD D (SEQ ID NO: 16, residues 62-117),
c. a sequence at least 85% identical to the amino acid sequence of IgBD A (SEQ ID NO: 16, residues 120-175),
d. a sequence at least 85% identical to the amino acid sequence of IgBD B (SEQ ID NO: 16, residues 178-233),
e. a sequence at least 85% identical to the amino acid sequence of IgBD C (SEQ ID NO: 16, residues 236-291),
and
ii. one or more first mutation(s) in each of the at least one of a-e, wherein the one or more first mutation(s) disrupt(s) binding in the Fe-binding site and wherein the one or more first mutation(s) is/are made in positions corresponding to amino acid positions in SEQ ID NO: 16 selected from positions 3, 8, 9, 64, 69, 70, 122, 127, 128, 180, 185, 186, 238, 243, 244, and
iii. one or more second mutation(s) in each of the at least one of a-e, wherein the one or more second mutation(s) disrupt(s) binding in the Fab-binding site and wherein the one or more second mutation(s), where applicable, is/are made in positions corresponding to amino acid positions in SEQ ID NO: 16 selected from positions 34, 35, 37, 38, 41, 95, 96, 98, 99, 102, 153, 154, 156, 157, 160, 211, 212, 214, 215, 218, 269, 270, 272, 273, and 276,
iv. wherein, optionally, none of the at least one of a-e, where applicable, comprise a mutation, which disrupts binding in the Fc-binding site, in positions corresponding to both of amino acid positions 7+8, 68+69, 126+127, 184+185, and 242+243 of SEQ ID NO: 16, and
v. wherein none of the at least one of a-e, where applicable, comprise a mutation, which disrupts binding in the Fab-binding site, in positions corresponding to both of amino acid positions 34+35 and 95+96 and 153+154 and 211+212, and 269+270 of SEQ ID NO: NO: 16,
wherein the polypeptide is unable to bind to human IgG and human von Willebrand factor and wherein the substitution(s) are preferably non-conservative substitutions.

107. The vaccine composition according to claim 106, wherein the one or more second mutation(s) is/are made in at least or only
1 or 2 or 3 or 4 or 5 of positions 34 and 96 and 154 and 212 and 270,
1 or 2 or 3 or 4 or 5 of positions 35 and 95 and 154 and 212 and 270,
1 or 2 or 3 or 4 or 5 of positions 35 and 96 and 153 and 212 and 270,
1 or 2 or 3 or 4 or 5 of positions 35 and 96 and 154 and 211 and 270,
1 or 2 or 3 or 4 or 5 of positions 35 and 96 and 154 and 212 and 269,
1 or 2 or 3 or 4 or 5 of positions 34 and 95 and 154 and 212 and 270,
1 or 2 or 3 or 4 or 5 of positions 34 and 96 and 153 and 212 and 270,
1 or 2 or 3 or 4 or 5 of positions 34 and 96 and 154 and 211 and 270,
1 or 2 or 3 or 4 or 5 of positions 34 and 96 and 154 and 212 and 269,
1 or 2 or 3 or 4 or 5 of positions 35 and 95 and 153 and 212 and 270,
1 or 2 or 3 or 4 or 5 of positions 35 and 95 and 154 and 211 and 270,
1 or 2 or 3 or 4 or 5 of positions 35 and 95 and 154 and 212 and 269,
1 or 2 or 3 or 4 or 5 of positions 35 and 96 and 153 and 211 and 270,
1 or 2 or 3 or 4 or 5 of positions 35 and 96 and 153 and 212 and 269,
1 or 2 or 3 or 4 or 5 of positions 35 and 96 and 154 and 211 and 269,
1 or 2 or 3 or 4 or 5 of positions 34 and 95 and 153 and 212 and 270,
1 or 2 or 3 or 4 or 5 of positions 34 and 95 and 154 and 211 and 270,
1 or 2 or 3 or 4 or 5 of positions 34 and 95 and 154 and 212 and 269,
1 or 2 or 3 or 4 or 5 of positions 34 and 96 and 153 and 211 and 270,
1 or 2 or 3 or 4 or 5 of positions 34 and 96 and 153 and 212 and 269,
1 or 2 or 3 or 4 or 5 of positions 34 and 96 and 154 and 211 and 269,
1 or 2 or 3 or 4 or 5 of positions 35 and 95 and 153 and 211 and 270,
1 or 2 or 3 or 4 or 5 of positions 35 and 95 and 153 and 212 and 269,
1 or 2 or 3 or 4 or 5 of positions 35 and 96 and 153 and 211 and 269,
1 or 2 or 3 or 4 or 5 of positions 34 and 95 and 153 and 211 and 270,
1 or 2 or 3 or 4 or 5 of positions 34 and 95 and 153 and 212 and 269,
1 or 2 or 3 or 4 or 5 of positions 34 and 95 and 154 and 211 and 269,
1 or 2 or 3 or 4 or 5 of positions 34 and 96 and 153 and 211 and 269,
1 or 2 or 3 or 4 or 5 of positions 35 and 95 and 153 and 211 and 269,
1 or 2 or 3 or 4 or 5 of positions 35 and 95 and 154 and 211 and 269,
1 or 2 or 3 or 4 or 5 of positions 34 and 95 and 153 and 211 and 269, or
1 or 2 or 3 or 4 or 5 of positions 35 and 96 and 154 and 212 and 270.

108. The vaccine composition according to claim 106 or 107, wherein the one or more second mutation(s) is/are made at least or only in a position corresponding to a position in SEQ ID NO: 16 selected from the group consisting of 34, 35, 95, 96, 153, 154, 211, 212, 269, and 270.

109. The vaccine composition according to claim 108, wherein the one or more second mutation(s) is/are made at least or only in a position corresponding to a position in SEQ ID NO: 16 selected from the group consisting of 34, 35, 95, 96, 153, 154, 211, 212, 269, and 270.

110. The vaccine composition according to claim 106 or 107, wherein the one or more second mutation(s) are made at least or only in positions corresponding to positions in SEQ ID NO: 16 selected from the group consisting of
34 and 95,
34 and 96,
34 and 153,
34 and 154,
34 and 211,
34 and 212,
34 and 269,
34 and 270,
35 and 95,
35 and 96,
35 and 153,
35 and 154,
35 and 211,
35 and 212,
35 and 269,
35 and 270,
95 and 153,
95 and 154,
95 and 211,
95 and 212,
95 and 269,
95 and 270,
96 and 153,
96 and 154,
96 mmd 211,
96 and 212,
96 and 269,
96 and 270,
153 and 211,
153 and 212,
153 and 269,
153 and 270,
154 and 211,
154 and 212,
154 and 269,
154 and 270,
211 and 269,
211 and 270,
212 and 269, and
212 and 270.

111. The vaccine composition according to claim 106 or 107, wherein the one or more second mutation(s) are made at least or only in positions corresponding to positions in SEQ ID NO: 16 selected from the group consisting of
34 and 95 and 153,
34 and 95 and 154,
34 and 95 and 211,
34 and 95 and 212,
34 and 95 and 269,
34 and 95 and 270,
35 and 95 and 153,
35 and 95 and 154,
35 and 95 and 211,
35 and 95 and 212,
35 and 95 and 269,
35 and 95 and 270,
34 and 96 and 153,
34 and 96 and 154,
34 and 96 and 211,
34 and 96 and 212,
34 and 96 and 269,
34 and 96 and 270,
35 and 96 and 153,
35 and 96 and 154,
35 and 96 and 211,
35 and 96 and 212,
35 and 96 and 269,
35 and 96 and 270,
34 and 153 and 211,
34 and 153 and 212,
34 and 154 and 269,
34 and 154 and 270,
35 and 153 and 211,
35 and 153 and 212,
35 and 154 and 269,
35 and 154 and 270,
34 and 211 and 269,
34 and 212 and 270,
35 and 211 and 269,
35 and 212 and 270,
95 and 153 and 211,
95 and 153 and 212,
95 and 153 and 269,
95 and 153 and 270,
95 and 154 and 211,
95 and 154 and 212,
95 and 154 and 269,
95 and 154 and 270,
96 and 153 and 211,
96 and 153 and 212,
96 and 153 and 269,
96 and 153 and 270,
96 and 154 and 211,
96 and 154 and 212,
96 and 154 and 269,
96 and 154 and 270,
95 and 211 and 269,
95 and 211 and 270,
95 and 212 and 269,
95 and 212 and 270,
96 and 212 and 269,
96 and 212 and 270,
96 and 212 and 269,
96 and 212 and 270,
153 and 211 and 269,
153 and 212 and 269,
153 and 211 and 270,
153 and 212 and 270,
154 and 211 and 269,
154 and 212 and 269,
154 and 211 and 270, and
154 and 212 and 270.

112. The vaccine composition according to claim 106 or 107, wherein the one or more second mutation(s) are made at least or only in positions corresponding to positions in SEQ ID NO: 16 selected from the group consisting of
95 and 153 and 211 and 269,
95 and 153 and 211 and 270,
95 and 153 and 212 and 269,
95 and 153 and 212 and 270,
95 and 154 and 211 and 269,
95 and 154 and 211 and 270,
95 and 154 and 212 and 269,
95 and 154 and 212 and 270,
96 and 153 and 211 and 269,
96 and 153 and 211 and 270,
96 and 153 and 212 and 269,
96 and 153 and 212 and 270,
96 and 154 and 211 and 269,
96 and 154 and 211 and 270,
96 and 154 and 212 and 269,
96 and 154 and 212 and 270,
34 and 153 and 211 and 269,
34 and 153 and 211 and 270,
34 and 153 and 212 and 269,
34 and 153 and 212 and 270,
34 and 154 and 211 and 269,
34 and 154 and 211 and 270,
34 and 154 and 212 and 269,
34 and 154 and 212 and 270,
35 and 153 and 211 and 269,
35 and 153 and 211 and 270,
35 and 153 and 212 and 269,
35 and 153 and 212 and 270,
35 and 154 and 211 and 269,
35 and 154 and 211 and 270,
35 and 154 and 212 and 269,
35 and 154 and 212 and 270,
34 and 95 and 211 and 269,
34 and 95 and 211 and 270,
34 and 95 and 212 and 269,
34 and 95 and 212 and 270,
34 and 96 and 211 and 269,
34 and 96 and 211 and 270,
34 and 96 and 212 and 269,
34 and 96 and 212 and 270,
35 and 95 and 211 and 269,
35 and 95 and 211 and 270,
35 and 95 and 212 and 269,
35 and 95 and 212 and 270,
35 and 96 and 211 and 269,
35 and 96 and 211 and 270,
35 and 96 and 212 and 269,
35 and 96 and 212 and 270,
34 and 95 and 153 and 269,
34 and 95 and 153 and 270,
34 and 95 and 154 and 269,
34 and 95 and 154 and 270,
34 and 96 and 153 and 269,
34 and 96 and 153 and 270,
34 and 96 and 154 and 269,
34 and 96 and 154 and 270,
35 and 95 and 153 and 269,
35 and 95 and 153 and 270,
35 and 95 and 154 and 269,
35 and 95 and 154 and 270,
35 and 96 and 153 and 269,
35 and 96 and 153 and 270,
35 and 96 and 154 and 269,
35 and 96 and 154 and 270,
34 and 95 and 153 and 211,
34 and 95 and 153 and 212,
34 and 95 and 154 and 211,
34 and 95 and 154 and 212,
34 and 96 and 153 and 211,
34 and 96 and 153 and 212,
34 and 96 and 154 and 211,
34 and 96 and 154 and 212,
35 and 95 and 153 and 211,
35 and 95 and 153 and 212,
35 and 95 and 154 and 211,
35 and 95 and 154 and 212,
35 and 96 and 153 and 211,
35 and 96 and 153 and 212,
35 and 96 and 154 and 211, and
35 and 96 and 154 and 212.

113. The vaccine composition according to claim 106 or 107, wherein the one or more second mutation(s) are made at least or only in positions corresponding to positions in SEQ ID NO: 16 selected from the group consisting of
34 and 96 and 154 and 212 and 270,
35 and 95 and 154 and 212 and 270,
35 and 96 and 153 and 212 and 270,
35 and 96 and 154 and 211 and 270,
35 and 96 and 154 and 212 and 269,
34 and 95 and 154 and 212 and 270,
34 and 96 and 153 and 212 and 270,
34 and 96 and 154 and 211 and 270,
34 and 96 and 154 and 212 and 269,
35 and 95 and 153 and 212 and 270,
35 and 95 and 154 and 211 and 270,
35 and 95 and 154 and 212 and 269,
35 and 96 and 153 and 211 and 270,
35 and 96 and 153 and 212 and 269,
35 and 96 and 154 and 211 and 269,
34 and 95 and 153 and 212 and 270,
34 and 95 and 154 and 211 and 270,
34 and 95 and 154 and 212 and 269,
34 and 96 and 153 and 211 and 270,
34 and 96 and 153 and 212 and 269,
34 and 96 and 154 and 211 and 269,
35 and 95 and 153 and 211 and 270,
35 and 95 and 153 and 212 and 269,
35 and 96 and 153 and 211 and 269,
34 and 95 and 153 and 211 and 270,
34 and 95 and 153 and 212 and 269,
34 and 95 and 154 and 211 and 269,
34 and 96 and 153 and 211 and 269,
35 and 95 and 153 and 211 and 269,
35 and 95 and 154 and 211 and 269,
34 and 95 and 153 and 211 and 269, and
35 and 96 and 154 and 212 and 270.

114. The vaccine composition according to claim 106 wherein the one or more first mutation(s) is/are made at least or only in a position corresponding to a position in SEQ ID NO: 16 selected from 3, 8, 9, 69, 127, 185, and 243.

115. The vaccine composition according to claim 114, wherein the one or more first mutation(s) are made in at least or only in positions corresponding to positions in SEQ ID NO: 16 selected from
3 and 8,
3 and 9,
3 and 69,
3 and 127,
3 and 185,
3 and 243,
8 and 9,
8 and 69,
8 and 127,
8 and 185,
8 and 243,
9 and 69,
9 and 127,
9 and 185,
9 and 243,
69 and 127,
69 and 185,
69 and 243,
127 and 185,
127 and 243,
185 and 243,
3 and 8 and 9,
3 and 8 and 69,
3 and 8 and 127,
3 and 8 and 185,
3 and 8 and 243,
3 and 9 and 69,
3 and 9 and 127,
3 and 9 and 185,
3 and 9 and 243,
3 and 69 and 127,
3 and 69 and 185,
3 and 69 and 243,
3 and 127 and 185,
3 and 127 and 243,
3 and 185 and 243,
8 and 9 and 69,
8 and 9 and 127,
8 and 9 and 185,
8 and 9 and 243,
8 and 69 and 127,
8 and 69 and 185,
8 and 69 and 243,
8 and 127 and 185,
8 and 127 and 243,
8 and 185 and 243,
9 and 69 and 127,
9 and 69 and 185,
9 and 69 and 243,
9 and 127 and 185,
9 and 127 and 243,
9 and 185 and 243,
69 and 127 and 185,
69 and 127 and 243,
69 and 185 and 243,
127 and 185 and 243,
3 and 8 and 9 and 69,
3 and 8 and 9 and 127,
3 and 8 and 9 and 185,
3 and 8 and 9 and 243,
3 and 8 and 69 and 127,
3 and 8 and 69 and 185,
3 and 8 and 69 and 243,
3 and 8 and 127 and 185,
3 and 8 and 127 and 243,
3 and 8 and 185 and 243,
3 and 9 and 69 and 127,
3 and 9 and 69 and 185,
3 and 9 and 69 and 243,
3 and 9 and 127 and 185,
3 and 9 and 127 and 243,
3 and 9 and 185 and 243,
3 and 69 and 127 and 185,
3 and 69 and 127 and 243,
3 and 69 and 185 and 243,
3 and 127 and 185 and 243,
8 and 9 and 69 and 127,
8 and 9 and 69 and 185,
8 and 9 and 69 and 243,
8 and 9 and 127 and 185,
8 and 9 and 127 and 243,
8 and 9 and 185 and 243,
8 and 69 and 127 and 185,
8 and 69 and 127 and 243,
8 and 69 and 185 and 243,
8 and 127 and 185 and 243,
9 and 69 and 127 and 185,
9 and 69 and 127 and 243,
9 and 69 and 185 and 243,
9 and 127 and 185 and 243,
69 and 127 and 185 and 243,
3 and 8 and 9 and 69 and 127,
3 and 8 and 9 and 69 and 185,
3 and 8 and 9 and 69 and 243,
3 and 8 and 9 and 127 and 185,
3 and 8 and 9 and 127 and 243,
3 and 8 and 9 and 185 and 243,
3 and 8 and 69 and 127 and 185,
3 and 8 and 69 and 127 and 243,
3 and 8 and 69 and 185 and 243,
3 and 8 and 127 and 185 and 243,
3 and 9 and 69 and 127 and 185,
3 and 9 and 69 and 127 and 243,
3 and 9 and 69 and 185 and 243,
3 and 9 and 127 and 185 and 243,
3 and 69 and 127 and 185 and 243,
8 and 9 and 69 and 127 and 185,
8 and 9 and 69 and 127 and 243,
8 and 9 and 69 and 185 and 243,
8 and 9 and 127 and 185 and 243,
8 and 69 and 127 and 185 and 243,
9 and 69 and 127 and 185 and 243,
3 and 8 and 9 and 69 and 127 and 185,
3 and 8 and 9 and 69 and 127 and 243,
3 and 8 and 9 and 69 and 185 and 243,
3 and 8 and 9 and 127 and 185 and 243,
3 and 8 and 69 and 127 and 185 and 243,
3 and 9 and 69 and 127 and 185 and 243,
8 and 9 and 69 and 127 and 185 and 243, and
3 and 8 and 9 and 69 and 127 and 185 and 243.

116. The vaccine composition according to claim 106, wherein the first and/or second mutation(s) is/are non-conservative substitution(s).

117. The vaccine composition according to claim 106, wherein at least one of the one or more first mutation(s) is/are as in claim 114, and wherein at least one of the one or more first mutation(s), which is/are as in claim 114, is a mutation to lysine (K).

118. The vaccine composition according to claim 117, wherein all of the one or more first mutation(s), which is/are as in claim 114, are a mutation to lysine (K).

119. The vaccine composition according to claim 106, wherein at least one of the one or more second mutation(s) is/are as in claim 109, and wherein at least one of the one or more second mutation(s), which is/are as in claim 109, is a mutation to alanine (A) or arginine (R).

120. The vaccine composition according to claim 119, wherein all of the one or more second mutation(s), which is/are as in claim 109, are a mutation to alanine (A) or arginine (R).

121. The vaccine composition according to claim 106, wherein the first mutation is according to claim 117 and the second mutation is according to claim 119, or wherein the first mutation is according to claim 118 and the second mutation is according to claim 119, or wherein the first mutation is according to claim 117 and the second mutation is according to claim 120, or wherein the first mutation is according to claim 118 and the second mutation is according to claim 120.

122. The vaccine composition according to claim 94, wherein the variant of the SpA polypeptide has at least 85% sequence identity with the sequence of SEQ ID NO: 16.

123. The vaccine composition according to claim 94, wherein the SpA polypeptide can induce antibodies that block the ability of wildtype SpA to bind to IgG and von Willebrand factor.

124. The vaccine composition according to claim 94, comprising the variant of the LukE polypeptide according to claim 1 b), which variant:
i. has at least 85% sequence identity with SEQ ID NO: 30, and
ii. does not comprise a signal peptide, which signal peptide corresponds to residues 1-28 of SEQ ID NO: 29.

125. The vaccine composition according to claim 124, wherein the LukE polypeptide consists of SEQ ID NO: 30.

126. The vaccine composition according to claim 124 or 125, wherein the LukE polypeptide can induce antibodies that block leukotoxicity of the functional mature wildtype LukE.

127. The vaccine composition according to claim 94, comprising the variant of the Aur polypeptide according to claim 94 d), which variant;
i. has at least 85% sequence identity with the amino acid sequence of SEQ ID NO: 34, and
ii. comprises one or more amino acid substitutions in the HEXXH catalytic domain, where the HEXXH catalytic domain corresponds to amino acid positions 352-356 of SEQ ID NO: 34,
wherein the Aur polypeptide has reduced catalytic capacity and wherein the substitution(s) are preferably non-conservative.

128. The vaccine composition according to claim 127, wherein, in the variant of the Aur polypeptide, the conserved cysteine (C), corresponding to amino acid position 479 of SEQ ID NO: 34, is substituted.

129. The vaccine composition according to claim 128, wherein the conserved cysteine is substituted with serine(S).

130. The vaccine composition according to any one of claims 127-129, wherein the glutamic acid residue (E) in the HEXXH catalytic domain, corresponding to amino acid position 353 of SEQ ID NO: 34, is substituted, preferably non-conservatively.

131. The vaccine composition according to claim 130, wherein the glutamic acid in the HEXXH catalytic domain is substituted with alanine (A).

132. The vaccine composition according to claim 127, wherein the variant of the Aur polypeptide consists of or comprises SEQ ID NO: 35.

133. The vaccine composition according to claim 127, wherein the variant of the Aur polypeptide comprises a sequence with at least 85% sequence identity with amino acid sequence 210-509 of SEQ ID NO: 34.

134. The vaccine composition according to claim 127, wherein the variant of the Aur polypeptide shows reduced ability to participate in forming antigen complexes as compared to wildtype Aur.

135. The vaccine composition according to claim 127, wherein the Aur polypeptide can induce antibodies that block the catalytic activities of wildtype Aur.

136. The vaccine composition according to claim 94, further comprising a variant of the amino acid sequence of N-acetylmuramoyl-L-alanine amidase (SAR2723), which variant has at least 85% sequence identity with the amino acid sequence of SEQ ID NO: 38, and
a. comprises one or more amino acid substitutions in the amidase active site TXEXX domain corresponding to amino acid residues 384-388 in SEQ ID NO: 37, and/or
b. comprises one or more substitutions in the amidase active site LXDYX domain corresponding to amino acid residues 409-413 in SEQ ID NO: 37, and/or
c. comprises a substitution of a conserved cysteine corresponding to position 513 in SEQ ID NO: 37,
wherein the SAR2723 polypeptide has reduced catalytic capacity and wherein the substitution(s) are preferably non-conservative.

137. The vaccine composition according to claim 136, in which the SAR2723 polypeptide comprises the following of features i: a only, b only, c only, a and b only, a and c only, b and c only, or a, b and c.

138. The vaccine composition according to any one of claim 136 or 137, wherein the conserved cysteine is substituted with serine(S).

139. The vaccine composition according to claim 136, wherein the glutamic acid residue (E) in the active site TXEXX domain, corresponding to amino acid position 386 of SEQ ID NO: 37, is substituted, for instance non-conservatively, preferably with glutamine (Q).

140. The vaccine composition according to claim 136, wherein the aspartic acid residue (D) in the active site LXDYS domain corresponding to amino acid position 411 of SEQ ID NO: 37, is substituted, such as non-conservatively, and preferably with asparagine (N).

141. The vaccine composition according to claim 136, wherein the variant of the amino acid sequence of SAR2723 consists of or comprises SEQ ID NO: 39.

142. The vaccine composition according to claim 136, wherein the SAR2723 polypeptide can induce antibodies that block the catalytic activities of wildtype SAR2723 or that interfere with bacterial cell wall development.

143. The vaccine composition according to claim 94, which comprises at least 3 of a-d as defined in claim 1.

144. The vaccine composition according to claim 94, which comprises
a and b;
a and c;
a and d;
a, b, and c;
a, b, and d;
a, c, and d; or
a, b, c, and d,
as defined in claim 1.

145. The vaccine composition according to any one of claims 94 and 143-144, which further comprises at least one polypeptide selected from
e) a SAR2723 polypeptide or a variant thereof, where said variant preferably exhibits reduced or no catalytic activity, and which can preferably induce antibodies that block the catalytic activity of native SAR2723,
f) a SAR2753 polypeptide or a variant, where said variant preferably exhibits reduced or no lipase activity and which can preferably induce antibodies that block the lipase activity of native SAR2753,
g) a SAR0992 (HtrA2) polypeptide or a variant thereof, and
h) a SAR0280 (EsaA) polypeptide of a variant thereof, where said variant can preferably induce antibodies that prevent EsxA/B secretion.

146. The vaccine composition according to claim 145, wherein
the SAR0992 polypeptide variant is a fragment or sequence variant of SEQ ID NO: 15 as disclosed in WO 2012/136653, in particular a fragment consisting of amino acid residues 1-409 of SEQ ID NO: 15 in WO 2012/136653, or a variant of SAR0992 that can induce antibodies that block catalytic activity of native SAR0992, such as a truncate of SAR0992 C-terminal relative to the transmembrane helix of SAR0992, or is a mutated version of SAR0992 comprising a substitution of the serine residue corresponding to the serine residue in position 619 in SEQ ID NO: 15 disclosed in WO 2012/136653, and/or is a mutated version of SAR0992, where the transmembrane helix is exchanged with a flexible linker or wherein the transmembrane helix is exchanged with a linker and the N- and C-terminal parts of SAR0992 flanking the linker are exchanged with each other, and/or
wherein the SAR0280 polypeptide variant is a fragment or sequence variant of SEQ ID NO: 13 as disclosed in WO 2012/136653 or is a fusion of the most N-terminal and the most C-terminal extracellular fragments of SAR0280, and/or wherein
the SAR2723 polypeptide variant is as defined in any one of claims 136-142 or a fragment or sequence variant of SEQ ID NO: 13 as disclosed in WO 2015/082536, and/or wherein
the SAR2753 polypeptide variant is a fragment or sequence variant of SEQ ID NO: 14 as disclosed in WO 2015/082536 or a polypeptide comprised of the amino acid sequence SEQ ID NO: 42.

147. The vaccine composition according to claim 145, which comprises at least 2 of e-h.

148. The vaccine composition according to claim 145, which comprises at least 3 of e-h.

149. The vaccine composition according to claim 145, which comprises
e and f;
e and g;
e and h;
f and g;
f and h;
g and h;
e, f, and g;
e, f, and h;
˜ e, g, and h;
f, g, and h; or
e, f, g, and h.

150. A chimeric polypeptide comprising the selection of polypeptides defined in claim 94, wherein the amino acid sequence of the Hla variant and the amino acid sequence(s) of the polypeptide(s) of b-d, as defined in claim 94, are fused or connected via a linker.

151. The chimeric polypeptide according to claim 150, wherein the linker is flexible or rigid.

152. The chimeric polypeptide according to claim 150, wherein the flexible linker is GSGGGA (SEQ ID NO: 50) or GSGGGAGSGGGA (SEQ ID NO: 51) or wherein the rigid linker is KPEPKPAPAPKP (SEQ ID NO: 52).

153. The chimeric polypeptide according to any one of claims 150-152, which has an amino acid sequence selected from the group consisting of CHIM_LukE_mHla_H35L_FS (SEQ ID NO: 56), CHIM_mc2723 Hla_H3SL_t_FS (SEQ ID NO: 62), CHIM_LukE_Hla_H35L_t_FS (SEQ ID NO: 64), and CHIM_0992_Hla_H35L_t_FS (SEQ ID NO: 68).

154. A vaccine composition comprising at least one chimeric polypeptide according to claim 150, said vaccine composition further comprising a pharmaceutically acceptable carrier, vehicle or diluent, and further optionally an immunological adjuvant.

155. The vaccine composition according to claim 154, wherein the at least one chimeric polypeptide is CHIM_LukE_mHla_H35L_FS (SEQ ID NO: 56).

156. The vaccine composition according to claim 154 or 155, comprising at least or exactly 2 chimeric polypeptides according to claim 150.

157. The vaccine composition according to claim 154, which further comprises at least or exactly one further polypeptide selected from the polypeptides defined in claim 94.

158. The vaccine composition according to claim 157, wherein the further polypeptide is selected from the group consisting of SAR0992-1-409 (residues 1-409 of SEQ ID NO: 44) and SAR0280-28-820 (SEQ ID NO: 48).

159. The vaccine composition according to claim 154, which comprises chimeric polypeptides CHIM_LukE_mHla_H35L_FS (SEQ ID NO: $6) and CHIM_mc2716 mSpA_7_12_FS (SEQ ID NO: 57), and optionally the polypeptide consisting of amino acid residues 1-409 of SAR0992 (residues 1-409 of SEQ ID NO: 44).

160. The vaccine composition according to claim 159, which consists of chimeric polypeptides CHIM_LukE_mHla_H35L_FS (SEQ ID NO: 56) and CHIM_mc2716_mSpA_7_12_FS (SEQ ID NO: 57), and optionally the polypeptide consisting of amino acid residues 1-409 of SAR0992 (residues 1-409 of SEQ ID NO: 44) in a mixture with a pharmaceutically acceptable carrier, vehicle or diluent, and further optionally an immunological adjuvant.

161. The vaccine composition according to claim 160, which comprises an immunological adjuvant selected from the group consisting of AIOH, SLA-SE, and OMVs (outer membrane vesicles).

162. A nucleic acid fragment encoding the chimeric polypeptide according to claim 150, such as a DNA fragment or an RNA fragment.

163. A vector comprising the nucleic acid fragment according to claim 162.

164. The vector according to claim 163, which is an expression vector.

165. A transformed cell or virus, which comprises and is capable of expressing the nucleic acid fragment according to claim 162 or the vector according to claim 164.

166. An immunogenic composition comprising the nucleic acid fragment according to claim 162, the vector according to claim 163, or the transformed cell or virus according to claim 165 and a pharmaceutically acceptable carrier, vehicle or diluent, and optionally an immunogenic adjuvant.

167. An immunogenic composition comprising (a) nucleic acid fragment(s), vector(s) or transformed cell(s) or virus that is/are capable of expressing the selection of polypeptides defined in claim 94, and a pharmaceutically acceptable carrier, vehicle or diluent, and optionally an immunogenic adjuvant.

168. A method for inducing immunity in an animal by administering at least once an immunogenically effective amount of the vaccine composition according to claim 94, the chimeric polypeptide according to claim 150, the nucleic acid fragment according to claim 162, the vector according to claim 163, the transformed cell or virus according to claim 165, or the immunogenic composition according to claim 166, so as to induce adaptive immunity against S. aureus in the animal.

169. The method according to claim 168, wherein, when the chimeric polypeptide according to claim 150, or a composition comprising said chimeric polypeptide is administered, the animal receives between 0.5 and 5,000 μg of the chimeric polypeptide according to any one of claims 150-153 per administration.

170. The method according to claim 168, wherein the animal receives a first priming administration comprising of said chimeric polypeptide and one or more booster administrations comprising said chimeric polypeptide.

171. The method according to claim 168, wherein the animal is a human being.

172. The method according to claim 168, wherein the administration is for the purpose of inducing protective immunity against S. aureus.

173. The method according to claim 172, wherein the protective immunity is effective in reducing the risk of attracting infection with S. aureus or is effective in treating or ameliorating infection with S. aureus.

174. The method according to claim 168, wherein the administration is for the purpose of inducing antibodies specific for S. aureus and wherein said antibodies or B-lymphocytes producing said antibodies are subsequently recovered from the animal.

175. The method according to claim 168, wherein the administration is for the purpose of inducing antibodies specific for S. aureus and wherein B-lymphocytes producing said antibodies are subsequently recovered from the animal and need for preparation of monoclonal antibodies.
